(19) United States
(12) Patent Application Publication
(10) Pub. No.: US 2002/0019030 A1
(43) Pub. Date: Feb. 14, 2002

(54) 25324, 50287, 28899, 47007, and 42967 transferase family members and uses therefor

(76) Inventors: Rachel Meyers, Newton, MA (US); Kyle J. MacBeth, Boston, MA (US); Laura Rudolph-Owen, Jamaica Plain, MA (US)

Correspondence Address: Intellectual Property Group, MILLENNIUM PHARMACEUTICALS, INC., 75 SIDNEY STREET, CAMBRIDGE, MA, 02139, US

(21) Appl. No.: 09/798,029

(22) Filed: Feb. 27, 2001

Related U.S. Application Data
(60) Provisional application No. 60/185,711, filed on Feb. 29, 2000.

(51) Int. Cl. C12Q 1/68; C07H 21/04; C12N 9/10

(52) U.S. Cl. 435/69.1; 435/193; 435/6; 435/325; 435/320.1; 536/23.2

(57) ABSTRACT

The invention provides isolated nucleic acids molecules, designated transferase nucleic acid molecules, which encode novel transferase family members. The invention also provides antisense nucleic acid molecules, recombinant expression vectors containing transferase nucleic acid molecules, host cells into which the expression vectors have been introduced, and nonhuman transgenic animals in which a transferase gene has been introduced or disrupted. The invention still further provides isolated transferase proteins, fusion proteins, antigenic peptides and anti-transferase antibodies. Diagnostic methods utilizing compositions of the invention are also provided.

This application claims priority on U.S. Provisional Application Ser. No. 60/185,711, filed Feb. 29, 2000 which is relied on and incorporated herein by reference.

### FIELD OF THE INVENTION

The present invention relates to polynucleotides encoding human transferase proteins, and transferase proteins encoded by such polynucleotides.

### BACKGROUND OF THE INVENTION

Transferases catalyze the transfer of one molecular group from one molecule to another. Such molecular groups include phosphate, amino, methyl, acetyl, acyl, phosphatidyl, phosphoribosyl, among other groups.

Transferases that transfer amino groups are known as aminotransferases or transaminases. Aminotransferases are enzymes that catalyze the transfer of amino groups from amino to -keto acids. The -amino groups of the 20 commonly found L-amino acids are removed during oxidative degradation of the amino acids. Removal of the -amino groups is the first step in the catabolism of the amino acids, and is promoted by an aminotransferase. In these transamination reactions, the -amino group is transferred to the -carbon atom of -ketoglutarate, leaving behind the corresponding -keto acid analog of the amino acid. There is no net deamination in such reactions because the -ketoglutarate becomes aminated as the -amino acid is deaminated. The effect of transamination reactions is to collect the amino groups from many different amino acids in the form of only one chemical compound, namely, L-glutamate. Glutamate can then direct amino groups either into biosynthetic pathways or into a final sequence of reactions by which nitrogenous waste products are produced and then excreted.

Cells contain multiple aminotransferases, many of which are specific for -ketoglutarate as the amino group acceptor. Aminotransferases differ in their specificity for the other substrate (the L-amino acid that donates the amino group) and are named for the amino group donor. The reactions catalyzed by the aminotransferases are freely reversible.

Aminotransferases play a role in clinically significant physiological activities. For example, measurements of alanine aminotransferase and aspartate aminotransferase levels in blood serum is an important diagnostic procedure in medicine as an indicator of heart damage and to monitor recovery from the damage.

Another example of an aminotransferase is the enzyme kynurenine aminotransferase, known in the art as KAT, which catalyzes the biosynthesis of kynurenic acid (KYNA) from kynurenine (KYN) and is singularly responsible for the regulation of extracellular KYNA concentrations in the brain (J. Neurochem., 57, 533-540, 1991).

KYNA is an effective excitatory amino acid (EAA) receptor antagonist with a particularly high affinity to the glycine modulatory site of the N-methyl-D-aspartate (NMDA) receptor complex (J. Neurochem., 52, 1319-1328, 1989). As a naturally occurring brain metabolite (J. Neurochem., 51, 177-180, 1988 and Brain Res., 454, 164-169, 1988), KYNA probably serves as a negative endogenous modulator of cerebral glutamatergic function (Ann.

N.Y. Acad. Sci., vol. 648, p. 140-153, 1992).

EAA receptors and in particular NMDA receptors are known to play a central role in the function of the mammalian brain (J. C. Watkins and G. L. Collingridge--eds.--, In: The NMDA receptor, Oxford University press, Oxford, p. 242, 1989). For example, NMDA receptor activation is essential for cognitive processes, such as, for example, learning and memory (J. C.

Watkins and G. L. Collingridge--eds.--, In: The NMDA receptor, Oxford University press, Oxford, p. 137-151, 1989) and for brain development (Trends Pharmacol. Sci., 11, 290-296, 1990).

It follows that a reduction in NMDA receptor function will have detrimental consequences for brain physiology and, consequently, for the entire organism. For example, the decline in the number of NMDA receptors which occurs in the aged brain (Synapse, 6, 343-388, 1990) is likely associated with age-related disorders of cognitive functions.

In the brain, KYNA concentrations and the activity of KYNA's biosynthetic enzyme KAT show a remarkable increase with age (Brain Res. 558, 1-5, 1992 and Neurosci. Lett., 94, 145-150, 1988). KAT inhibitors, by providing an increase of the glutamatergic tone at the NMDA receptor, could therefore be particularly useful in situations where NMDA receptor function is insufficient and/or KAT activity and KYNA levels are abnormally enhanced. Hence they could be particularly useful in the treatment of the pathological consequences associated with the aging processes in the brain which are, for example, cognitive disorders including, e.g., attentional and memory deficits and vigilance impairments in the elderly.

KAT inhibitors may also be useful in the treatment of perinatal brain disorders which may be related to irregularities in the characteristic region specific pattern of postnatal KAT development (H. Baran and R. Schwarcz: Regional differences in the ontogenic pattern of KAT in the brain, Dev. Brain Res., 74, 283-286, 1993).

Aminotransferases share certain mechanistic features with pyridoxal-phosphate dependent enzymes, such as the covalent binding of the pyridoxal-phosphate group to a lysine residue. On the basis of sequence similarity, these various enzymes can be grouped into subfamilies. One of these, called class-I, comprises the following enzymes; aspartate aminotransferase (AAT), which catalyzes the reversible transfer of the amino group from L-aspartate to 2-oxoglutarate to form oxaloacetate and L-glutamate (In eukaryotes, there are two AAT isozymes: one is located in the mitochondrial matrix, the second is cytoplasmic. In prokaryotes, only one form of AAT is found (gene aspC); tyrosine aminotransferase which catalyzes the first step in tyrosine catabolism by reversibly transferring its amino group to 2-oxoglutarate to form 4-hydroxyphenylpyruvate and L-glutamate; aromatic aminotransferase involved in the synthesis of Phe, Tyr, Asp and Leu (gene tyrB); 1-aminocyclopropane-1-carboxylate synthase (ACC synthase) from plants, which catalyzes the first step in ethylene biosynthesis; Pseudomonas denitrificans cobC, which is involved in cobalamin biosynthesis; and yeast hypothetical protein YJL060w.

Another sub-family, called class-II, comprises the following enzymes: glycine acetyltransferase, which catalyzes the addition of acetyl-CoA to glycine to form 2-amino-3-oxobutanoate (gene kbl); 5-aminolevulinic acid synthase (delta-ALA synthase), which catalyzes the first step in heme biosynthesis via the Shemin (or C4) pathway, i.e. the addition of succinyl-CoA to glycine to form 5-aminolevulinate; 8-amino-7-oxononanoate synthase (7-KAP synthetase), a bacterial enzyme (gene bioF) which catalyzes an intermediate step in the biosynthesis of biotin, that is, the addition of 6-carboxy-hexanoyl-CoA to alanine to form 8-amino-7-oxononanoate; histidinol-phosphate aminotransferase, which catalyzes the eighth step in histidine biosynthetic pathway, that is the transfer of an amino group from 3-(imidazol-4-yl)-2-oxopropil phosphate to glutamic acid to form histidinol phosphate and 2-oxoglutarate; serine palmitoyltransferase from yeast (genes LCB 1 and LCB2), which catalyzes the condensation of palmitoyl-CoA and serine to form 3-ketosphinganine.

The sequence around the pyridoxal-phosphate attachment site of this class of enzyme is sufficiently conserved to allow the creation of a specific pattern.

The group of acyltransferases includes enzymes like bacterial malonyl CoA-acyl carrier protein transacylase and fatty acid synthase that are involved in fatty acid biosynthes. Also included are the polyketide synthases 6-methylsalicylic acid synthase and a multifunctional enzyme that involved in the biosynthesis of patulin and conidial green pigment synthase. This family also contains acyltransferases involved in phospholipid biosynthesis and includes tafazzin, the Barth syndrome gene.

The acetyltransferase (GNAT) family contains proteins with N-acetyltransferase functions. The GCN5-related N-acetyltransferase superfamily includes such enzymes as the histone acetyltransferases GCN5 and Hatl. The yeast GCN5 (yGCN5) transcriptional coactivator functions as a histone acetyltransferase (HAT) to promote transcriptional activation. The crystal structure of the yeast histone acetyltransferase Hat-acetyl coenzyme A (AcCoA) shows that Hat has an elongated, curved structure, and the AcCoA molecule is bound in a cleft on the concave surface of the protein, marking the active site of the enzyme. A channel of variable width and depth that runs across the protein is probably the binding site for the histone substrate. The central protein core associated with AcCoA binding that appears to be structurally conserved among a superfamily of N-acetyltransferases, including yeast histone acetyltransferase 1 and Serratia marcescens aminoglycoside 3-N-acetyltransferase.

Some detoxification reactions are catalyzed by enzymes that promote acetylation of aminoglycosides. Structural studies of these aminoglycoside-modifying enzymes may assist in the development of therapeutic agents that could circumvent antibiotic resistance. In addition, such studies may shed light on the development of antibiotic resistance and the evolution of different enzyme classes.

Another transferase, phosphotidyl transferase has been reported as being involved in the biosynthesis of phosphatidyl-scyllo-inositol found in barley, presumably in the transfer of the phosphotidyl group from one molecular entity to another. Carstensen, S. et al., Lipids 34(1):67-731993.

Phosphoribosyltransferases (PRT) are enzymes that catalyze the synthesis of beta-n-5′-monophosphates from phosphoribosylpyrophosphate (PRPP) and an enzyme specific amine. A number of PRT's are involved in the biosynthesis of purine, pyrimidine, and pyridine nucleotides, or in the salvage of purines and pyrimidines. These enzymes are: adenine phosphoribosyltransferase (APRT), which is involved in purine salvage; hypoxanthine-guanine or hypoxanthine phosphoribosyltransferase (HGPRT or HPRT), which are involved in purine salvage; orotate phosphoribosyltransferase (OPRT), which is involved in pyrimidine biosynthesis; amido phosphoribosyltransferase, which is involved in purine biosynthesis; xanthine-guanine phosphoribosyltransferase (XGPRT), which is involved in purine salvage. In the sequence of all of these enzymes there is a small conserved region which may be involved in the enzymatic activity and/or be part of the PRPP binding site.

### SUMMARY OF THE INVENTION

The present invention is based, at least in part, on the discovery of transferase family members, referred to herein as “transferase” or “25324, 50287, 28899, 47007, or 42967” nucleic acid and protein molecules. The transferase molecules of the present invention are useful as modulating agents, or as targets for developing modulating agents to regulate a variety of cellular processes facilitated by transferase molecules. Accordingly, in one aspect, this invention provides isolated nucleic acid molecules encoding transferase proteins or biologically active portions thereof, as well as nucleic acid fragments suitable as primers or hybridization probes for the detection of transferase-encoding nucleic acids.

One aspect of the invention relates to an aminotransferase as well as the nucleic acids that encode it. In particular, the 25324 aminotransferase has homology to kynurenine amninotransferase, also known as kynurenine/-aminoadipate aminotransferase or kynurenineoxoglutarate aminotransferase. In accordance with this aspect of the invention, the aminotransferase, or polynucleotides encoding it, may be used to catalyze the biosynthesis of kynurenic acid (KYNA) from kynurenine (KYN). Such use permits the production of KYNA for applications as an EAA receptor antagonist and to act as a negative endogenous modulator of cerebral glutamatergic function.

The 25324 aminotransferase of the invention may also be used in screens to identify new inhibitors of KYNA biosynthesis, which could be advantageous in cases where NMDA receptor function is insufficient and/or where biosynthetic activity or KYN levels are abnormally high. Such inhibitors would be expected to be of particular utility in treatment of cognitive disorders such as attention and memory deficits.

The 47007 transferase has homology to phosphotidyl transferase, which has been reported as being involved in the biosynthesis of phosphatidyl-scyllo-inositol. Carstensen, S. et al., Lipids 34(1):67-73 1993. In accordance with this aspect of the invention, the 47007 transferase, or polynucleotides encoding it, may be the human analog of the reported phosphotidyl transferase and may be used to catalyze reactions analogous to the biosynthesis of phosphatidol-scyllo-inositol.

In one embodiment, a transferase nucleic acid molecule of the invention is at least 50%, 55%, 60%, 65%, 70%, 75%, 80%, 85%, 90%, 95%, 98%, 99%, or more identical to the nucleotide sequence (e.g., to the entire length of the nucleotide sequence) shown in SEQ ID NO: 1, 3, 5, 7, or 9, or a complement thereof.

In another embodiment, the isolated nucleic acid molecule includes the nucleotide sequence shown SEQ ID NO: 1, 3, 5, 7, or 9, or a complement thereof In another embodiment, the 25324 nucleic acid molecule includes at least one fragment of at least 301 or 1754 nucleotides (e.g., 301 or 1754 contiguous nucleotides) of at least one nucleotide sequence of SEQ ID NO: 1 or a complement thereof; the 50287 nucleic acid molecule includes at least one fragment of at least 654 nucleotides (e.g., 654 contiguous nucleotides) of at least one nucleotide sequence of SEQ ID NO: 3 or a complement thereof; the 28899 nucleic acid molecule includes at least one fragment of at least 867 nucleotides (e.g., 867 contiguous nucleotides) of at least one nucleotide sequence of SEQ If) NO: 5 or a complement thereof; the 47007 nucleic acid molecule includes at least one fragment of at least 25 nucleotides (e.g., 25 contiguous nucleotides) of at least one nucleotide sequence of SEQ ID NO: 7 or a complement thereof; or the 42967 nucleic acid molecule includes at least one fragment of at least 25 nucleotides (e.g., 25 contiguous nucleotides) of at least one nucleotide sequence of SEQ if NO: 9 or a complement thereof.

In still another embodiment, a transferase nucleic acid molecule includes a nucleotide sequence encoding a protein having an amino acid sequence sufficiently homologous to the amino acid sequence of SEQ ID NO: 2, 4, 6, 8, or 10. In one embodiment, a transferase nucleic acid molecule includes a nucleotide sequence encoding a protein having an amino acid sequence at least 50%, 55%, 60%, 65%, 70%, 75%, 80%, 85%, 90%, 95%, 98% or more identical to the entire length of the amino acid sequence of SEQ ID NO: 2, 4, 6, 8, or 10.

In another embodiment, an isolated nucleic acid molecule encodes the amino acid sequence of human transferase. In yet another embodiment, the nucleic acid molecule includes a nucleotide sequence encoding a protein having the amino acid sequence of SEQ ID NO: 2, 4, 6,8,or 10.

In yet another embodiment, the 25324 nucleic acid molecule is, in length, at least 1275 nucleotides (e.g., 1275 contiguous nucleotides) of at least one nucleotide sequence of SEQ ID NO: 1 or a complement thereof; the 50287 nucleic acid molecule is at least 552 nucleotides (e.g., 552 contiguous nucleotides) of at least one nucleotide sequence of SEQ If) NO: 3 or a complement thereof; the 28899 nucleic acid molecule is at least 1128 nucleotides (e.g., 1128 contiguous nucleotides) of at least one nucleotide sequence of SEQ ID NO: 5 or a complement thereof; the 47007 nucleic acid molecule is at least 1269 nucleotides (e.g., 1269 contiguous nucleotides) of at least one nucleotide sequence of SEQ ID NO: 7 or a complement thereof; or the 42967 nucleic acid molecule is at least 519 nucleotides (e.g., 519 contiguous nucleotides) of at least one nucleotide sequence of SEQ ID NO: 9 or a complement thereof. In a further preferred embodiment, the nucleic acid molecule has the length set forth immediately above and encodes a protein having a transferase activity as described herein.

Another embodiment of the invention features nucleic acid molecules, preferably transferase nucleic acid molecules, which specifically detect transferase nucleic acid molecules relative to nucleic acid molecules encoding non-transferase proteins. For example, in one embodiment, such a nucleic acid molecule is at least 50, 60, 70, 80, 90, 100, 150, 200, 300, 400, 500, 549, 549-600, (for SEQ ID NO: 1, 3, 5, 7, 9) 600-650, 650-700, 700-750, 750-800, 800-850, 850-900, 900-950, 950-1000, (for SEQ ID NO: 1, 3, 5, 7)1000-1100, 1100-1200, 1200-1300 1300-1400, 1400-1500, 1500-1600, 1600-1700, 1700-1800, (for SEQ ID NO: 1, 5, 7) 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, 4400-4500, 4500-4600, 4600-4700, 4700-4800, 4800-4900, 4900-5000, 5000-5100, 5100-5200, 5200-5300, 5300-5400, (for SEQ ID NO: 7) or more nucleotides in length and hybridizes under stringent conditions to a nucleic acid molecule comprising the nucleotide sequence shown in SEQ ID NO: 1, 3, 5, 7, or 9.

In other embodiments, the nucleic acid molecule encodes a naturally occurring allelic variant of a polypeptide comprising the amino acid sequence of SEQ ID NO: 2, 4, 6, 8, or 10, wherein the nucleic acid molecule hybridizes to a nucleic acid molecule comprising SEQ ID NO: 1, 3, 5, 7, or 9 under stringent conditions.

Another embodiment of the invention provides an isolated nucleic acid molecule which is antisense to a transferase nucleic acid molecule, e.g., the coding strand of a transferase nucleic acid molecule (SEQ ID NOs: 11-15 excluding the terminal codon).

In a related aspect, the invention provides a vector comprising a transferase nucleic acid molecule. In certain embodiments, the vector is a recombinant expression vector. In another embodiment, the invention provides a host cell containing a vector of the invention. In yet another embodiment, the invention provides a host cell containing a nucleic acid molecule of the invention. The invention also provides a method for producing a protein, preferably a transferase protein, by culturing in a suitable medium, a host cell, e.g., a mammalian host cell, such as a non-human mammalian cell, of the invention containing a recombinant expression vector, such that the protein is produced.

Another aspect of this invention features isolated or recombinant transferase proteins and polypeptides. In one embodiment, the isolated transferase protein includes at least one domain as shown in FIGS. 2, 3, 5, 6, 8, 9, 11, 12, 14, and 15.

In other embodiments, the transferase protein of the invention has an amino acid sequence at least about 50%, 55%, 60%, 65%, 70%, 75%, 80%, 85%, 90%, 95%, 98% or more identical to the amino acid sequence of SEQ ID NO: 2, 4, 6, 8, or 10. In another embodiment, the transferase protein includes at least one domain as shown in FIGS. 2, 3, 5, 6, 8, 9, 11, 12, 14, and 15, and has an amino acid sequence at least about 50%, 55%, 60%, 65%, 70%, 75%, 80%, 85%, 90%, 95%, 98% or more identical to the amino acid sequence of SEQ ID NO: 2, 4, 6, 8, or 10.

In another embodiment, the transferase proteins of the invention play a role in cell growth and cell processes facilitated by transferase proteins, e.g., the regulation of cell proliferation, differentiation, migration, and apoptosis; modulate angiogenic processes; are involved in controlling inflammation; or are involved in cardiovascular disorders.

In other embodiments, the transferase proteins of the invention are encoded by a nucleic acid molecule having a nucleotide sequence which hybridizes under stringent hybridization conditions to a nucleic acid molecule comprising the nucleotide sequence of SEQ ID NO: 1, 3, 5, 7, or 9.

In a further embodiment, the invention features an isolated transferase protein which is encoded by a nucleic acid molecule consisting of a nucleotide sequence at least about 50%, 55%, 60%, 65%, 70%, 75%, 80%, 85%, 90%, 95%, 98% or more identical to a nucleotide sequence of SEQ ID NO: 1, 3, 5, 7, or 9, or a complement thereof. This invention further features an isolated transferase protein which is encoded by a nucleic acid molecule consisting of a nucleotide sequence which hybridizes under stringent hybridization conditions to a nucleic acid molecule comprising the nucleotide sequence of SEQ ID NO: 1, 3, 5, 7, or 9, or a complement thereof. In still another embodiment, the transferase protein has the amino acid sequence of SEQ ID NO: 2, 4, 6, 8, or 10.

In another embodiment, the invention features fragments of the protein having the amino acid sequence of SEQ ID NO: 2, 4, 6, 8, or 10, wherein the fragment comprises at least 15 amino acids (e.g., contiguous amino acids) of the amino acid sequence of SEQ ID NO: 2, 4, 6, 8, or 10, and the fragment comprises preferably at least 20, 25, 30, 35, 40, 45, 50, 65, 100, 130, 160-170 (for SEQ ID NO: 2, 4, 6, 8, 10), 170-180 (for SEQ ID NO: 2, 4, 6, 8), 180-210, 210-230, 230-250, 250-265, 265-280, 280-300, 300-315, 315-330, 330-350, 350-375, (for SEQ ID NO: 2, 6, 8), 375-400, or 400-420, (for SEQ ID NO: 2, 8) amino acids.

The proteins of the present invention or portions thereof, e.g., biologically active portions thereof, can be operatively linked to a non-transferase polypeptide (e.g., heterologous amino acid sequences) to form fusion proteins. In addition, the transferase proteins or biologically active portions thereof can be incorporated into pharmaceutical compositions, which optionally include pharmaceutically acceptable carriers.

The invention further features antibodies, such as monoclonal or polyclonal antibodies, that specifically bind proteins of the invention, preferably transferase proteins.

In another aspect, the present invention provides a method for detecting the presence of a transferase nucleic acid molecule, protein or polypeptide in a biological sample by contacting the biological sample with an agent capable of detecting a transferase nucleic acid molecule, protein or polypeptide such that the presence of a transferase nucleic acid molecule, protein or polypeptide is detected in the biological sample.

In another aspect, the present invention provides a method for detecting the presence of transferase activity in a biological sample by contacting the biological sample with an agent capable of detecting an indicator of transferase activity such that the presence of transferase activity is detected in the biological sample.

In another aspect, the invention provides a method for modulating transferase activity comprising contacting a cell capable of expressing transferase with an agent that modulates transferase activity such that transferase activity in the cell is modulated. In one embodiment, the agent inhibits transferase activity. In another embodiment, the agent stimulates transferase activity. In one embodiment, the agent is an antibody that specifically binds to a transferase protein. In another embodiment, the agent modulates expression of transferase by modulating transcription of a transferase gene or translation of a transferase mRNA. In yet another embodiment, the agent is a nucleic acid molecule having a nucleotide sequence that is antisense to the coding strand of a transferase mRNA or a transferase gene.

Another aspect of the present invention features methods to treat a subject having a disorder characterized by aberrant transferase protein or nucleic acid expression or activity by administering an agent which is a transferase modulator to the subject. In one embodiment, the transferase modulator is a transferase protein. In another embodiment the transferase modulator is a transferase nucleic acid molecule. In yet another embodiment, the transferase modulator is a peptide, peptidomimetic, or other small molecule.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

Aberrant expression and/or activity of transferase molecules may mediate disorders associated with bone metabolism. “Bone metabolism” refers to direct or indirect effects in the formation or degeneration of bone structures, e.g., bone formation, bone resorption, etc., which may ultimately affect the concentrations in serum of calcium and phosphate. This term also includes activities mediated by transferase molecules effects in bone cells, e.g. osteoclasts and osteoblasts, that may in turn result in bone formation and degeneration. For example, transferase molecules may support different activities of bone resorbing osteoclasts such as the stimulation of differentiation of monocytes and mononuclear phagocytes into osteoclasts. Accordingly, transferase molecules that modulate the production of bone cells can influence bone formation and degeneration, and thus may be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyroidism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

The transferase nucleic acid and protein of the invention can be used to treat and/or diagnose a variety of immune disorders. Exemplary immune disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol/Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease.

Additional examples of hematopoietic disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosus, autoimmune thyroiditis, dermatitis (including atopic dermatitis and eczematous dermatitis), psoriasis, Sjögren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graftversus-host disease, cases of transplantation, and allergy such as, atopic allergy.

Examples of disorders involving the heart or “cardiovascular disorder” include, but are not limited to, a disease, disorder, or state involving the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. Examples of such disorders include hypertension, atherosclerosis, coronary artery spasm, congestive heart failure, coronary artery disease, valvular disease, arrhythmias, and cardiomyopathies.

Disorders which may be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolism, for example, fibrosis resulting from a storage disorder such as Gaucher's disease (lipid abnormalities) or a glycogen storage disease, Al-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein may be useful for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

Additionally, transferase may play an important role in the etiology of certain viral diseases, including but not limited to Hepatitis B, Hepatitis C and Herpes Simplex Virus (HSV). Modulators of transferase activity could be used to control viral diseases. The modulators can be used in the modulation, treatment and/or diagnosis of viral infected tissue or virus-associated tissue fibrosis, especially liver and liver fibrosis. Also, transferase modulators can be used in the modulation, treatment and/or diagnosis of virus-associated carcinoma, especially hepatocellular cancer.

Additionally, transferase may play an important role in the regulation of metabolism. Diseases of metabolic imbalance include, but are not limited to obesity, anorexia nervosa, cachexia, lipid disorders diabetes.

The transferase molecules provide novel diagnostic targets and therapeutic agents to control pain in a variety of disorders, diseases, or conditions which are characterized by a deregulated, e.g., upregulated or downregulated, pain response. For example, the transferase molecules provide novel diagnostic targets and therapeutic agents to control the exaggerated pain response elicited during various forms of tissue injury, e.g., inflammation, infection, and ischemia, usually referred to as hyperalgesia (described in, for example, Fields, H. L. (1987) Pain, New York: McGraw-Hill). Moreover, the transferase molecules provide novel diagnostic targets and therapeutic agents to control pain associated with muscoloskeletal disorders, e.g., joint pain, tooth pain, headaches, or pain associated with surgery.

The present invention also provides a diagnostic assay for identifying the presence or absence of a genetic alteration characterized by at least one of (i) aberrant modification or mutation of a gene encoding a transferase protein; (ii) mis-regulation of the gene; and (iii) aberrant post-translational modification of a transferase protein, wherein a wild-type form of the gene encodes a protein with a transferase activity.

In another aspect the invention provides a method for identifying a compound that binds to or modulates the activity of a transferase protein, by providing an indicator composition comprising a transferase protein having transferase activity, contacting the indicator composition with a test compound, and determining the effect of the test compound on transferase activity in the indicator composition to identify a compound that modulates the activity of a transferase protein.

Other features and advantages of the invention will be apparent from the following detailed description and claims.

### BRIEF DESCRIPTION OF THE DRAWINGS

FGS. 1a-b depict the cDNA sequence (SEQ ID NO: 1) and predicted amino acid sequence (SEQ ID NO: 2) of human 25324 transferase. The nucleotide sequence corresponds to the 1892 nucleic acids of SEQ ID NO: 1 which include nucleic acids 1-1275 of the coding region (SEQ ID NO: 11, not including the terminal codon), the 5′ UTR of 279 nucleic acids, and the 3′ UTR of 335 nucleic acids. The amino acid sequence corresponds to amino acids 1 to 425 of SEQ ID NO: 2.

FIG. 2 depicts a series of plots summarizing an analysis of the primary and secondary protein structure of human 25324. The particular algorithm used for each plot is indicated at the right hand side of each plot. The following plots are depicted: Gamier-Robson plots providing the predicted location of alpha-, beta-, and turn regions (Garnier et al. (1978) J. Mol. Biol. 120:97); Chou-Fasman plots providing the predicted location of alpha-, beta-, turn and coil regions (Chou and Fasman (1978) Adv. In Enzymol. Mol. 47:45-148); Kyte-Doolittle hydrophilicity/hydrophobicity plots (Kyte and Doolittle (1982) J. Mol. Biol. 157:105-132); Eisenberg plots providing the predicted location of alpha- and beta-amphipathic regions (Eisenberg et al. (1982) Nature 299:371-374); a Karplus-Schultz plot providing the predicted location of flexible regions (Karplus and Schulz (1985) Naturwissens-Chafen 72:212-213); a plot of the antigenic index (Jameson-Wolf) (Jameson and Wolf (1988) CABIOS 4:121-136); and a surface probability plot (Emini algorithm) (Emini et al. (1985) J. Virol. 55:836-839). The numbers corresponding to the amino acid sequence of human 25324 are indicated.

FIGS. 3 a-e are data generated using the 25324 protein. A hydropathy plot of human 25324 shows relative hydrophobic residues above the dashed horizontal line, and relative hydrophilic residues below the dashed horizontal line. The cysteine residues (cys) are indicated by short vertical lines just below the hydropathy trace. The numbers corresponding to the amino acid sequence of human 25324 are indicated. The signal peptide predictions and the transmembrane regions as predicted by MEMSTAT are also shown. Results from the Prosite database of protein families and domains identify biologically significant sites. PFAM search results depict alignments of an aminotransferase of class I and II domain of human 25324 with a consensus amino acid sequence derived from a hidden Markov model. The upper sequence is the consensus amino acid sequence, while the lower amino acid sequence corresponds to a portion of the amino acids of SEQ ID NO: 2. Finally, results from the ProDom protein domain database identify homologous domains. The lower sequence is the consensus amino acid sequence, while the upper amino acid sequence corresponds to a portion of SEQ ID NO: 2.

FIG. 4 depicts the cDNA sequence (SEQ ID NO: 3) and predicted amino acid sequence (SEQ ID NO: 4) of human 50287 transferase. The nucleotide sequence corresponds to the 1892 nucleic acids of SEQ ID NO: 3 which include nucleic acids 1-552 of the coding region (SEQ ID NO: 12, not including the terminal codon), the 5′ UTR of 183 nucleic acids, and the 3′ UTR of 263 nucleic acids. The amino acid sequence corresponds to amino acids 1 to 184 of SEQ ID NO: 4.

FIG. 5 depicts a series of plots summarizing an analysis of the primary and secondary protein structure of human 50287. The particular algorithm used for each plot is indicated at the right hand side of each plot. The following plots are depicted: Garnier-Robson plots providing the predicted location of alpha-, beta-, and turn regions (Ganier et al. (1978) J. Mol. Biol. 120:97); Chou-Fasman plots providing the predicted location of alpha-, beta-, turn and coil regions (Chou and Fasman (1978) Adv. In Enzymol. Mol. 47:45-148); Kyte-Doolittle hydrophilicity/hydrophobicity plots (Kyte and Doolittle (1982) J. MoL. Biol. 157:105-132); Eisenberg plots providing the predicted location of alpha- and beta-amphipathic regions (Eisenberg et al. (1982) Nature 299:371-374); a Karplus-Schultz plot providing the predicted location of flexible regions (Karplus and Schulz (1985) Naturwissens-Chafen 72:212-213); a plot of the antigenic index (Jameson-Wolf) (Jameson and Wolf (1988) CABIOS 4:121-136); and a surface probability plot (Emini algorithm) (Emini et al. (1985) J. Virol. 55:836-839). The numbers corresponding to the amino acid sequence of human 50287 are indicated.

FIGS. 6 a-d are data generated using the 50287 protein. A hydropathy plot of human 50287 shows relative hydrophobic residues above the dashed horizontal line, and relative hydrophilic residues below the dashed horizontal line. The cysteine residues (cys) are indicated by short vertical lines just below the hydropathy trace. The location of the transmembrane domains, and the extracellular and intracellular loops is also indicated. The numbers corresponding to the amino acid sequence of human 50287 are indicated. The signal peptide predictions and the transmembrane regions as predicted by MEMSTAT are also shown. Results from the Prosite database of protein families and domains identify biologically significant sites. PFAM search results depict alignments of an acetyltransferase (GNAT) domain of human 50287 with a consensus amino acid sequence derived from a hidden Markov model. The upper sequence is the consensus amino acid sequence, while the lower amino acid sequence corresponds to a portion of the amino acids of SEQ ID NO: 4. Finally, results from the ProDom protein domain database identify homologous domains. The lower sequence is the consensus amino acid sequence, while the upper amino acid sequence corresponds to a portion of SEQ ID NO: 4.

FIGS. 7 a-b depict the cDNA sequence (SEQ ID NO: 5) and predicted amino acid sequence (SEQ ID NO: 6) of human 28899 transferase. The nucleotide sequence corresponds to the 1832 nucleic acids of SEQ ID NO: 5 which include nucleic acids 1-1128 of the coding region (SEQ ID NO: 13, not including the terminal codon), the 5′ UTR of 191 nucleic acids, and the 3′ UTR of 510 nucleic acids. The amino acid sequence corresponds to amino acids 1 to 376 of SEQ ID NO: 6.

FIG. 8 depicts a series of plots summarizing an analysis of the primary and secondary protein structure of human 28899. The particular algorithm used for each plot is indicated at the right hand side of each plot. The follo wing plots are depicted: Gamier-Robson plots providing the predicted location of alpha-, beta-, and turn regions (Gamier et al. (1978) J. Mol. Biol. 120:97); Chou-Fasman plots providing the predicted location of alpha-, beta-, turn and coil regions (Chou and Fasman (1978) Adv. In Enzymol. Mol. 47:45-148); Kyte-Doolittle hydrophilicity/hydrophobicity plots (Kyte and Doolittle (1982) J. Mol. Biol. 157:105-132); Eisenberg plots providing the predicted location of alpha- and beta-amphipathic regions (Eisenberg et al. (1982) Nature 299:371-374); a KarplusSchultz plot providing the predicted location of flexible regions (Karplus and Schulz (1985) Naturwissens-Chafen 72:212-213); a plot of the antigenic index (Jameson-Wolf) (Jameson and Wolf (1988) CABIOS 4:121-136); and a surface probability plot (Emini algorithm) (Emini et al. (1985) J. Virol. 55:836-839). The numbers corresponding to the amino acid sequence of human 28899 are indicated.

FIGS. 9 a-f are data generated using the 28899 protein. A hydropathy plot of human 28899 shows relative hydrophobic residues above the dashed horizontal line, and relative hydrophilic residues below the dashed horizontal line. The cysteine residues (cys) are indicated by short vertical lines just below the hydropathy trace. The location of the transmembrane domains, and the extracellular and intracellular loops is also indicated. The numbers corresponding to the amino acid sequence of human 28899 are indicated. Also depicted is the prediction of protein subcellular localization sites using PSORT software. The signal peptide predictions and the transmembrane regions as predicted by MEMSTAT are also shown. Results from the Prosite database of protein families and domains identify biologically significant sites. PFAM search results depict alignments of an acyltransferase domain of human 28899 with a consensus amino acid sequence derived from a hidden Markov model. The upper sequence is the consensus amino acid sequence, while the lower amino acid sequence corresponds to a portion of the amino acids of SEQ ID NO: 6. Finally, results from the ProDom protein domain database identify homologous domains. The lower sequence is the consensus amino acid sequence, while the upper amino acid sequence corresponds to a portion of SEQ ID NO: 6.

FIGS. 10 a-c depict the cDNA sequence (SEQ ID NO: 7) and predicted amino acid sequence (SEQ ID NO: 8) of human 47007 transferase. The nucleotide sequence corresponds to the 5426 nucleic acids of SEQ ID NO: 7 which include nucleic acids 1-1269 of the coding region (SEQ ID NO: 14, not including the terminal codon), the 5′ UTR of 1392 nucleic acids, and the 3′ UTR of 2762 nucleic acids. The amino acid sequence corresponds to amino acids 1 to 423 of SEQ ID NO: 8.

FIG. 11 depicts a series of plots summarizing an analysis of the primary and secondary protein structure of human 47007. The particular algorithm used for each plot is indicated at the right hand side of each plot. The following plots are depicted: Gamier-Robson plots providing the predicted location of alpha-, beta-, and turn regions (Gamier et al. (1978) J. Mol. Biol. 120:97); Chou-Fasman plots providing the predicted location of alpha-, beta-, turn and coil regions (Chou and Fasman (1978)Adv. In Enzymol. Mol. 47:45-148); Kyte-Doolittle hydrophilicity/hydrophobicity plots (Kyte and Doolittle (1982)J. Mol. Biol. 157:105-132); Eisenberg plots providing the predicted location of alpha- and beta-amphipathic regions (Eisenberg et al. (1982) Nature 299:371-374); a Karplus-Schultz plot providing the predicted location of flexible regions (Karplus and Schulz (1985) Naturwissens-Chafen 72:212-213); a plot of the antigenic index (Jameson-Wolf) (Jameson and Wolf (1988) CABIOS 4:121-136); and a surface probability plot (Emini algorithm) (Emini et al. (1985) J. Virol. 55:836-839). The numbers corresponding to the amino acid sequence of human 47007 are indicated.

FIGS. 12a-d are data generated using the 47007 protein. A hydropathy plot of human 47007 shows relative hydrophobic residues above the dashed horizontal line, and relative hydrophilic residues below the dashed horizontal line. The cysteine residues (cys) are indicated by short vertical lines just below the hydropathy trace. The numbers corresponding to the amino acid sequence of human 47007 are indicated. Also depicted is the prediction of protein subcellular localization sites using PSORT software. The signal peptide predictions and the transmembrane regions as predicted by MEMSTAT are also shown. Results from the Prosite database of protein families and domains identify biologically significant sites. Finally, results from the ProDom protein domain database identify homologous domains. The lower sequence is the consensus amino acid sequence, while the upper amino acid sequence corresponds to a portion of SEQ ID NO: 8.

FIG. 13 depict the cDNA sequence (SEQ ID NO: 9) and predicted amino acid sequence (SEQ ID NO: 10) of human 42967 transferase. The nucleotide sequence corresponds to the 602 nucleic acids of SEQ ID NO: 9 which include nucleic acids 1-519 of the coding region (SEQ ID NO: 15, not including the terminal codon), the 5′ UTR of 25 nucleic acids, and the 3′ UTR of 55 nucleic acids. The amino acid sequence corresponds to amino acids 1 to 173 of SEQ ID NO: 10.

FIG. 14 depicts a series of plots summarizing an analysis of the primary and secondary protein structure of human 42967. The particular algorithm used for each plot is indicated at the right hand side of each plot. The following plots are depicted: Gamier-Robson plots providing the predicted location of alpha-, beta-, and turn regions (Garmier et al. (1978) J. Mol. Biol. 120:97); Chou-Fasman plots providing the predicted location of alpha-, beta-, turn and coil regions (Chou and Fasman (1978) Adv. In Enzymol. Mol. 47:45-148); Kyte-Doolittle hydrophilicity/hydrophobicity plots (Kyte and Doolittle (1982) J. Mol. Biol. 157:105-132); Eisenberg plots providing the predicted location of alpha- and beta-amphipathic regions (Eisenberg et al. (1982) Nature 299:371-374); a Karplus-Schultz plot providing the predicted location of flexible regions (Karplus and Schulz (1985) Naturwissens-Chafen 72:212-213); a plot of the antigenic index (Jameson-Wolf) (Jameson and Wolf(1988) CABIOS4:121-136); and a surface probability plot (Emini algorithm) (Emini et al. (1985) J. Virol. 55:836-839). The numbers corresponding to the amino acid sequence of human 42967 are indicated.

FIGS. 15 a-c are data generated using the 42967 protein. A hydropathy plot of human 42967 shows relative hydrophobic residues above the dashed horizontal line, and relative hydrophilic residues below the dashed horizontal line. The N-glycosylation sites (Ngly) are indicated by short vertical lines just below the hydropathy trace. The numbers corresponding to the amino acid sequence of human 42967 are indicated. Also depicted is the prediction of protein subcellular localization sites using PSORT software. The signal peptide predictions and the transmembrane regions as predicted by MEMSTAT are also shown. Results from the Prosite database of protein families and domains identify biologically significant sites. PFAM search results depict alignments of a phosphribosyl transferase domain of human 42967 with a consensus amino acid sequence derived from a hidden Markov model. The upper sequence is the consensus amino acid sequence, while the lower amino acid sequence corresponds to a portion of the amino acids of SEQ ID NO: 10. Finally, results from the ProDom protein domain database identify homologous domains. The lower sequence is the consensus amino acid sequence, while the upper amino acid sequence corresponds to a portion of SEQ ID NO: 10.

FIG. 16 depicts variable expression of 50827 in a xenograph panel.

FIG. 17 is a bar graph depicting the relative expression of 50827 RNA relative to a no template control in a panel of human tissues or cells, including but not limited to heart, brain, glial, breast, ovary, prostate, epithelial, colon, colon tumor, liver, liver fibrosis, lung, lung tumor, spleen, tonsil, lymph node, among others, detected using real-time quantitative RT-PCR Taq Man analysis. The graph indicates significant expression in lung tumor.

FIG. 18 is a breast model bar graph depicting the relative expression of 50827 RNA relative to a no template control in a panel of human normal breast cell lines and breast carcinoma cells detected using real-time quantitative RT-PCR Taq Man analysis. The highest level of expression was found in the MCF-7 breast carcinoma cells.

FIG. 19 is an oncology phase II panel bar graph depicting the expression of 28899 RNA relative to a no template control showing an increased expression in 6/6 breast tumor samples in comparison with normal breast tissue; showing an increased expression in 2/4 ovary tumor samples in comparison with normal ovar y tissue; and showing an increased expression in 5/7 various lung tumor samples in comparison with normal lung tissue, which expression was detected using Taq Man analysis.

FIG. 20 is a bar graph depicting the relative expression of 28899 RNA relative to a no template control in a panel of human tissues or cells, including but not limited to heart, kidney, skeletal muscle, brain, nerve, dorsal root ganglia, glial, breast, ovary, prostate, epithelial, colon, colon tumor, lung, lung tumor, liver, liver fibrosis, spleen, tonsil, lymph node, among others, detected using real-time quantitative RT-PCR Taq Man analysis. The graph indicates significant expression in normal brain cortex.

FIG. 21 depicts the relative expression of 28899 RNA relative to a no template control in a panel of human ovarian cell lines detected using real-time quantitative RT-PCR Taq Man analysis. The highest level of expression was found in the MDA 127 N ovarian epithelial cells.

FIG. 22 is a breast model bar graph depicting the relative expression of 28899 RNA relative to a no template control in a panel of human normal breast cell lines and breast carcinoma cells detected using real-time quantitative RT-PCR Taq Man analysis. The highest level of expression was found in the MCF-10AT 3B and MCF-10A m25 cells and MCF-7 breast carcinoma cells.

FIG. 23 is a lung model panel bar graph depicting the relative expression of 28899 RNA relative to a no template control in a panel of human normal and carcinoma lung cell lines detected using real-time quantitative RT-PCR Taq Man analysis. The highest level of expression was found in H522 (AC), H69 (SCLC), H345 Mock, and H345 VIP cancer cell lines.

FIG. 24 is an angiogenic panel depicting the expression of 47007 RNA relative to a no template control showing a decreased expression in 6/6 brain tumor samples in comparison with normal breast tissue; and showing high expression in fetal adrenal tissues, which expression was detected using Taq Man analysis.

FIG. 25 is a bar graph depicting the relative expression of 47007 RNA relative to a no template control in a panel of human tissues or cells, including but not limited to heart, kidney, skeletal muscle, brain, nerve, dorsal root ganglia, glial, breast, ovary, prostate, epithelial, colon, colon tumor, lung, lung tumor, liver, liver fibrosis, spleen, tonsil, lymph node, among others, detected using real-time quantitative RT-PCR Taq Man analysis. The graph indicates significant expression in normal brain cortex and prostate epithelial cells.

FIG. 26 is a vessel panel bar graph depicting the relative expression of 47007 RNA relative to a no template control in a panel of human normal and diseased blood vessels detected using real-time quantitative RT-PCR Taq Man analysis. The highest level of expression was found in aortic smooth muscle cells (SMC) (late) and confluent human umbilical vein epithelial cells (HUVEC).

### DETAILED DESCRIPTION OF THE INVENTION

The present invention is based, at least in part, on the discovery of novel transferase family members, referred to herein as “transferase” nucleic acid and protein molecules.

The transferase molecules of the present invention are predicted to modulate and facilitate cell proliferation, differentiation, motility, and apoptosis. Thus, the transferase molecules of the present invention may play a role in cellular growth signaling mechanisms. As used herein, the term “cellular growth signaling mechanism” includes signal transmissions from cell receptors, e.g., growth factor receptors, which regulate one or more of the following: 1) cell transversal through the cell cycle, 2) cell differentiation, 3) cell migration and patterning, 4) programmed cell death, 5) angiogenic processes, 6) inflammation, and 7) cardiovascular processes. Throughout development and in the adult organism, cell fate and activity is determined, in part, by extracellular and intracellular stimuli, e.g., growth factors, angiogenic factors, chemotactic factors, neurotrophic factors, cytokines, and hormones. These stimuli act on their target cells by initiating signal transduction cascades that alter the pattern of gene expression and metabolic activity so as to mediate the appropriate cellular response. The transferase molecules of the present invention are predicted to be involved in the initiation or modulation of cellular signal transduction pathways that modulate cell growth, differentiation, migration and/or apoptosis. Thus, the transferase molecules, by participating in cellular growth signaling mechanisms, may modulate cell behavior and act as therapeutic agents for controlling cellular proliferation, differentiation, migration, and apoptosis.

Altered expression of factors (e.g., a transferase molecule) involved in the regulation of signaling pathways associated with cell growth, differentiation, migration, and apoptosis can lead to perturbed cellular proliferation, which in turn can lead to cellular proliferative and/or differentiative disorders. As used herein, a “cellular proliferative disorder” includes a disorder, disease, or condition characterized by a deregulated, e.g., upregulated or downregulated, growth response. As used herein, a “cellular differentiative disorder” includes a disorder, disease, or condition characterized by aberrant cellular differentiation. Thus, the transferase molecules can act as novel diagnostic targets and therapeutic agents for controlling cellular proliferative and/or differentiative disorders. Examples of cellular proliferative and/or differentiative disorders include cancer, e.g., carcinoma, sarcoma, or leukemia; and disorders involving aberrant angiogenesis and/or vascularity, e.g., tumor angiogenesis and metastasis, diabetic retinopathy, macular degeneration, psoriasis, endometriosis, Grave's disease, ischemic disease (e.g., atherosclerosis), and chronic inflammatory diseases (e.g., rheumatoid arthritis).

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

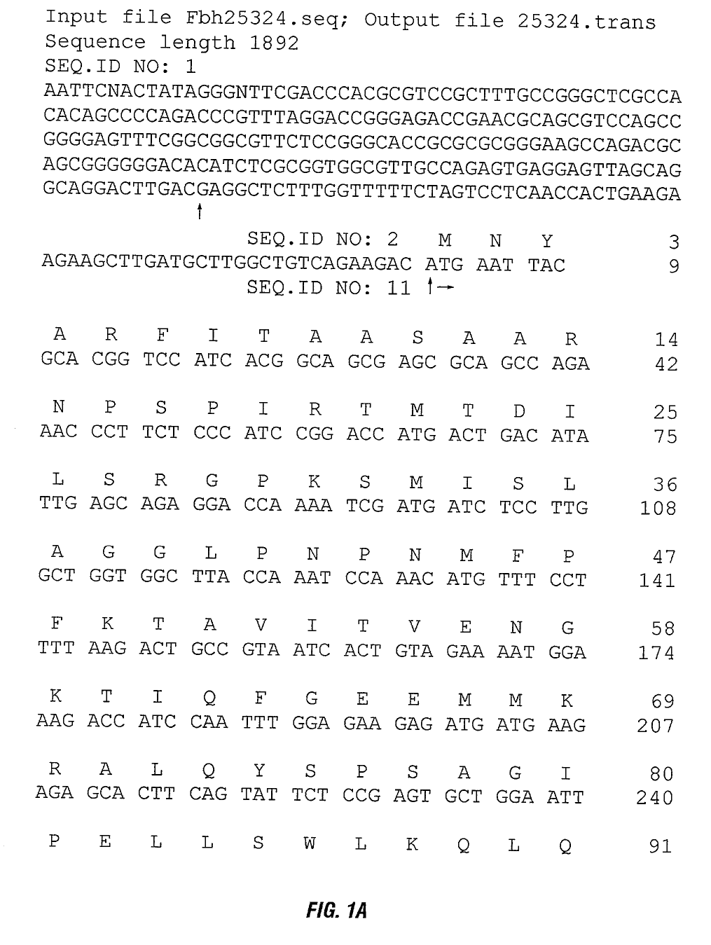

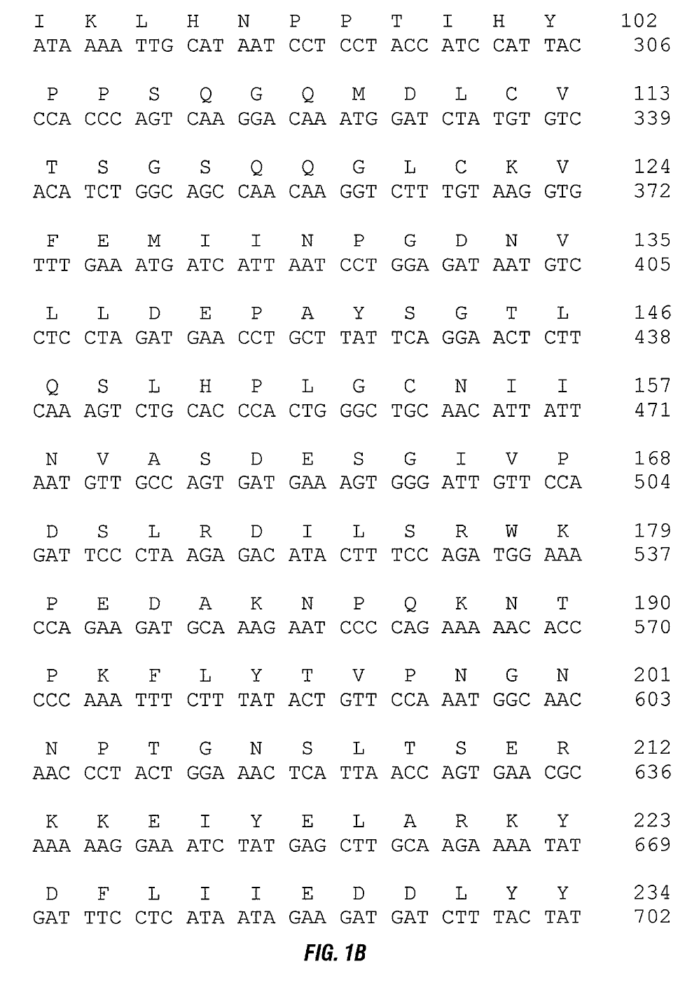

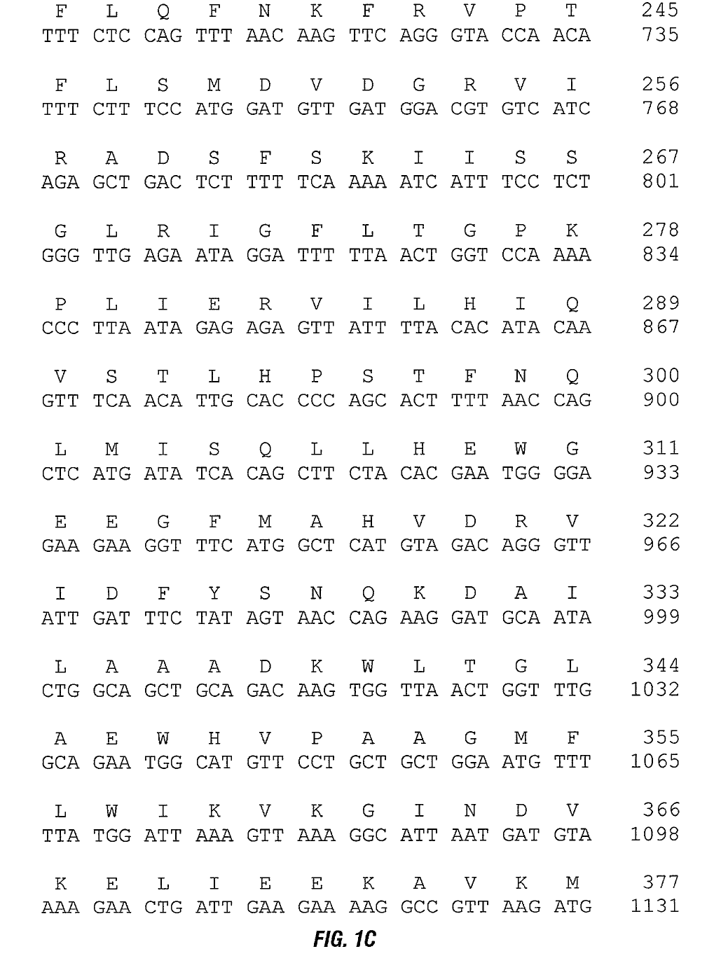

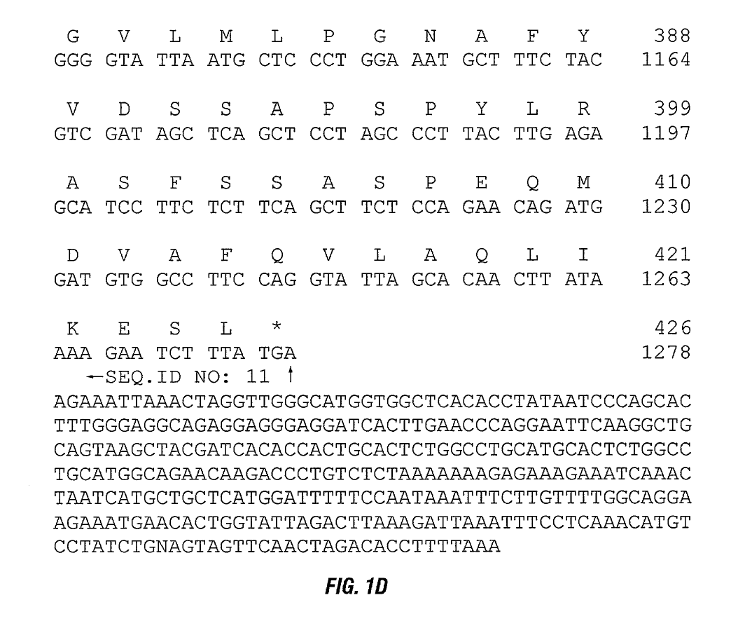

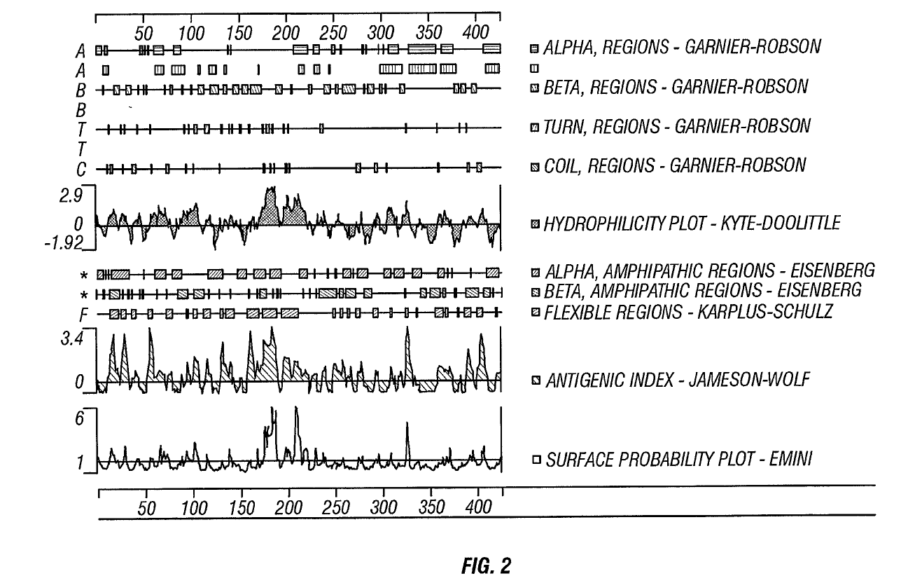

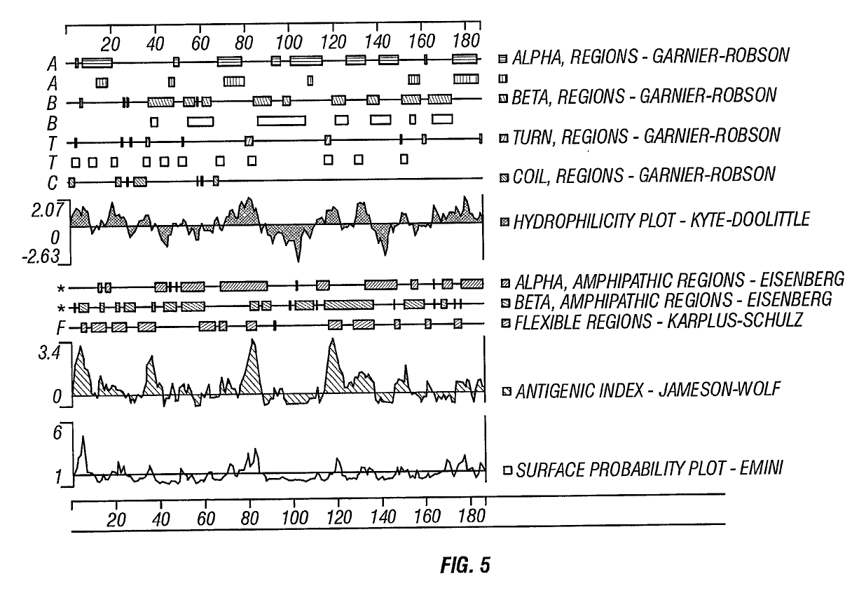

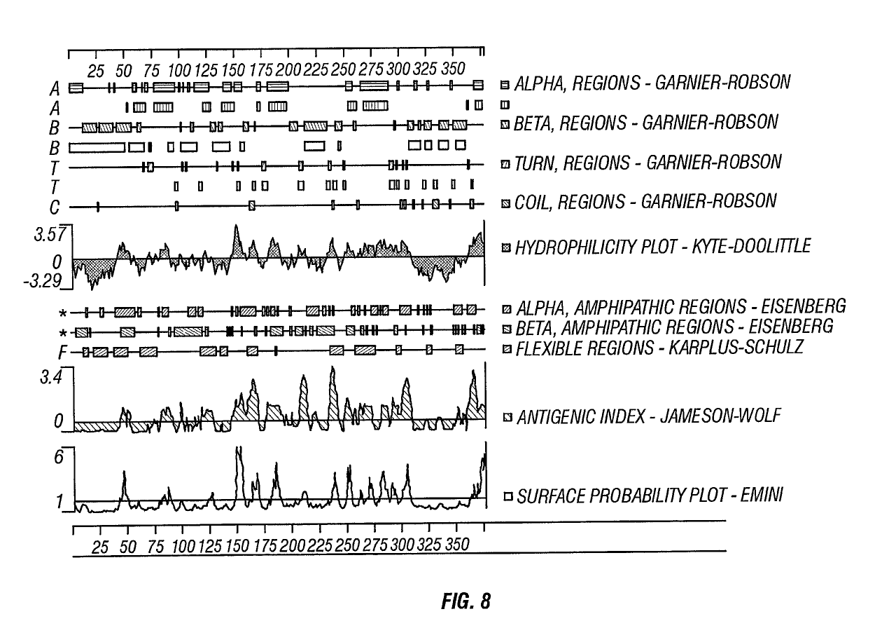

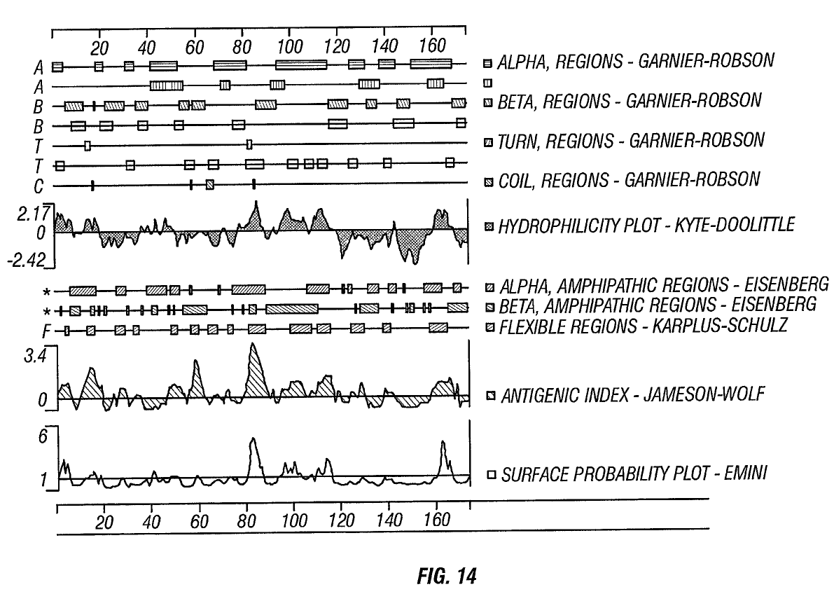

For example, members of the transferase family of proteins include at least one domain as shown in FIGS. 2, 3, 5, 6, 8, 9, 11, 12, 14, and 15 in the protein molecule or the nucleic acid molecule encoding the protein molecule.

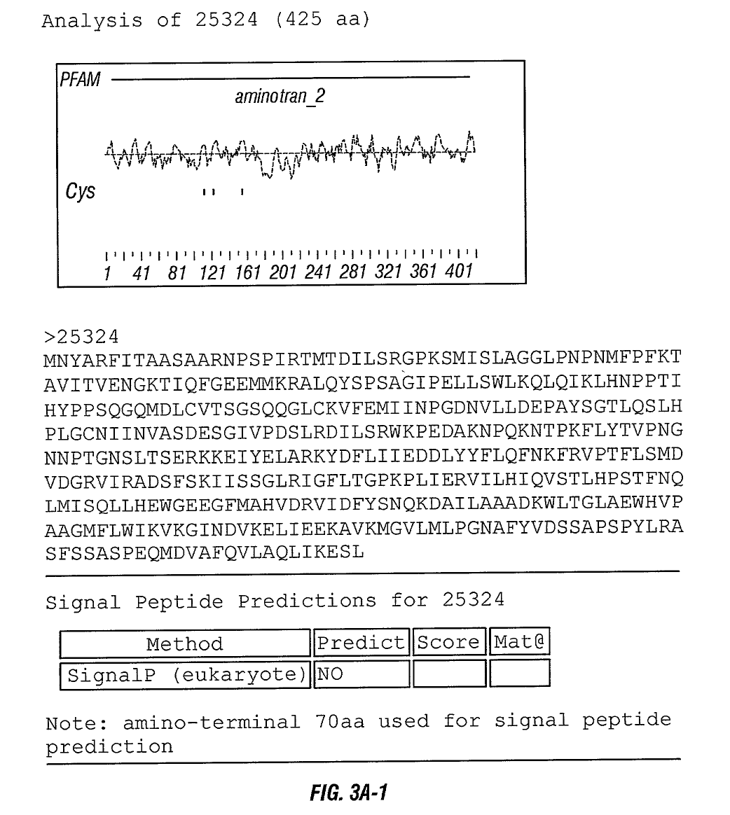

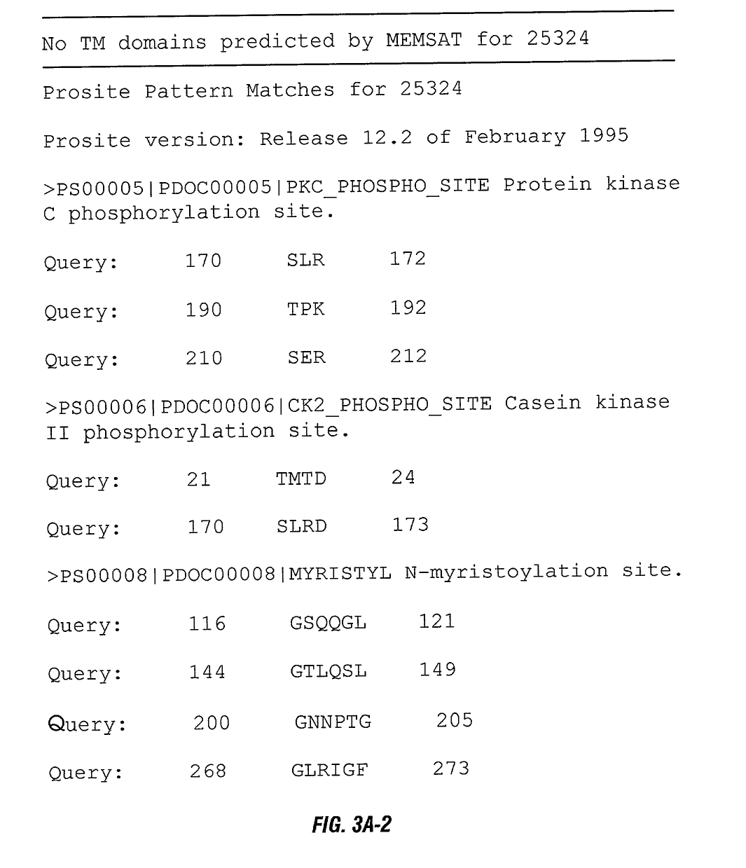

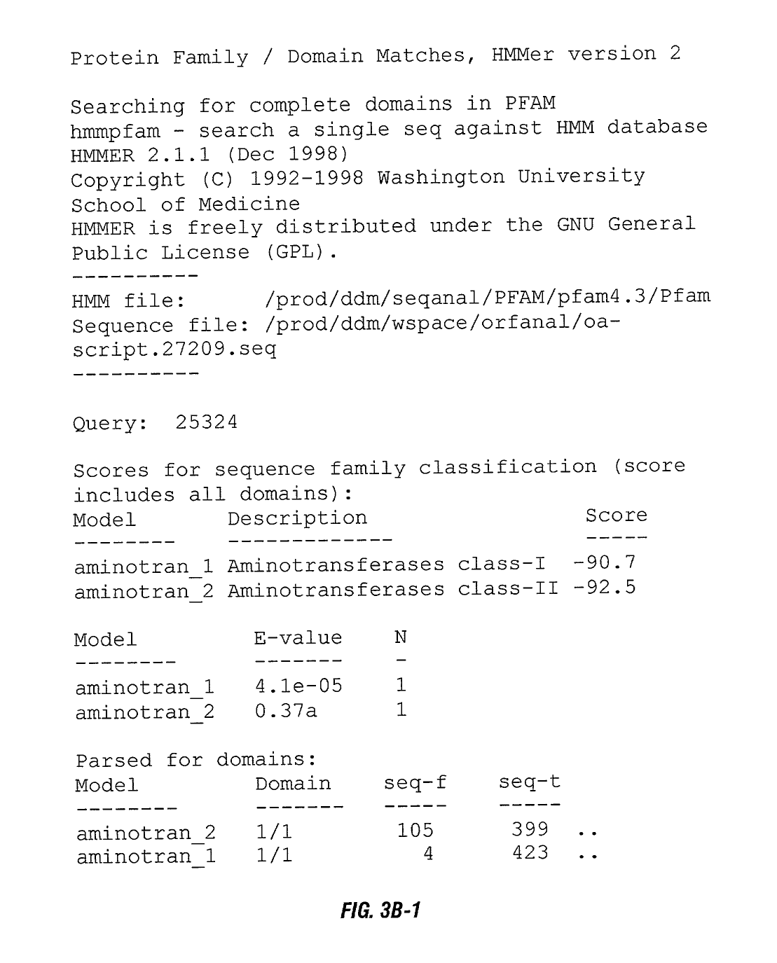

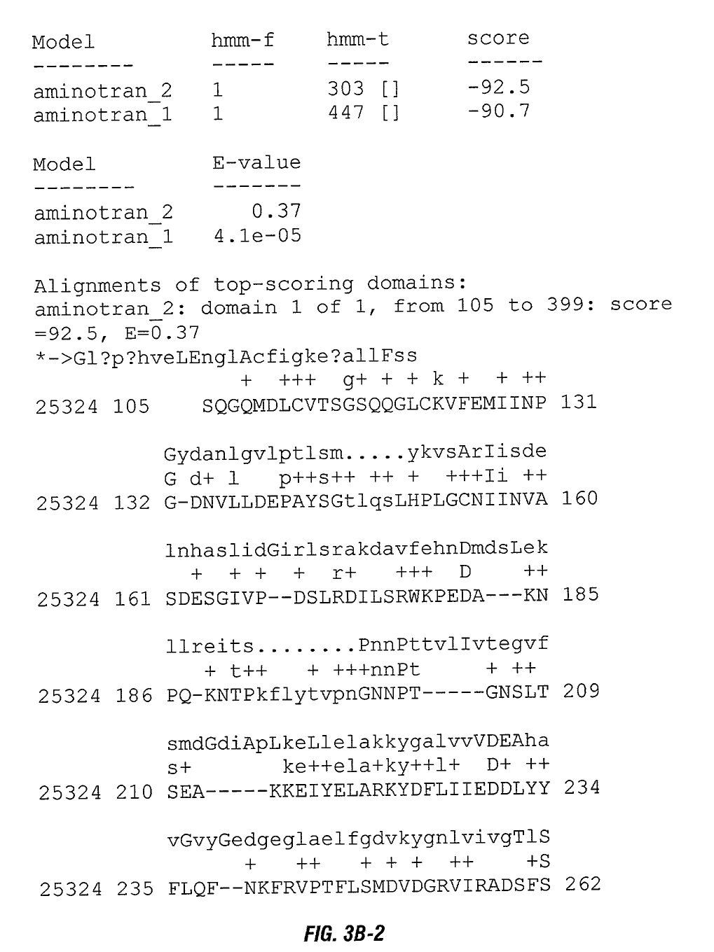

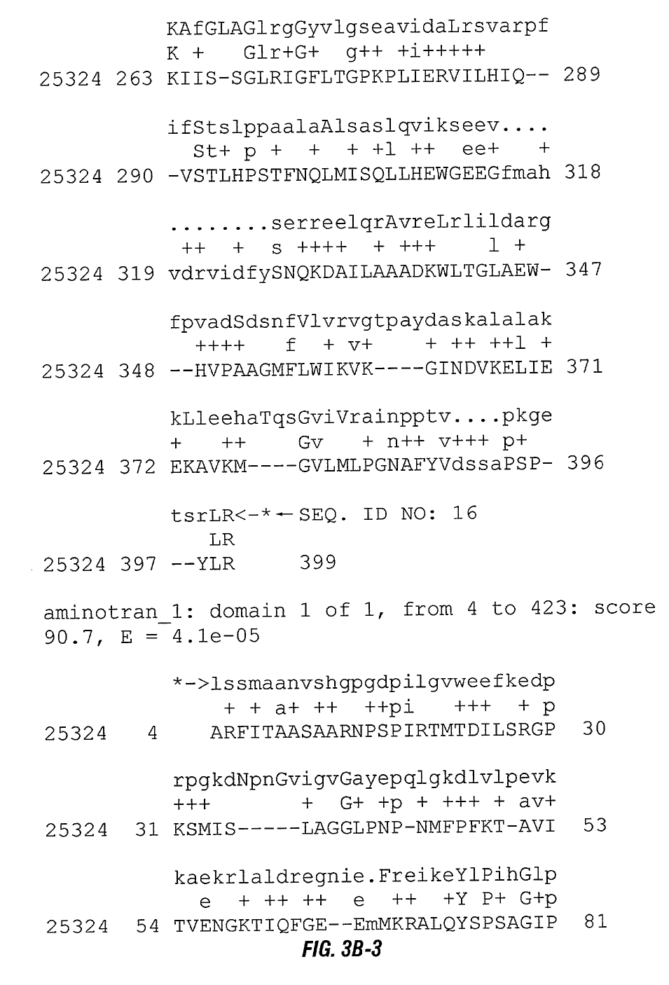

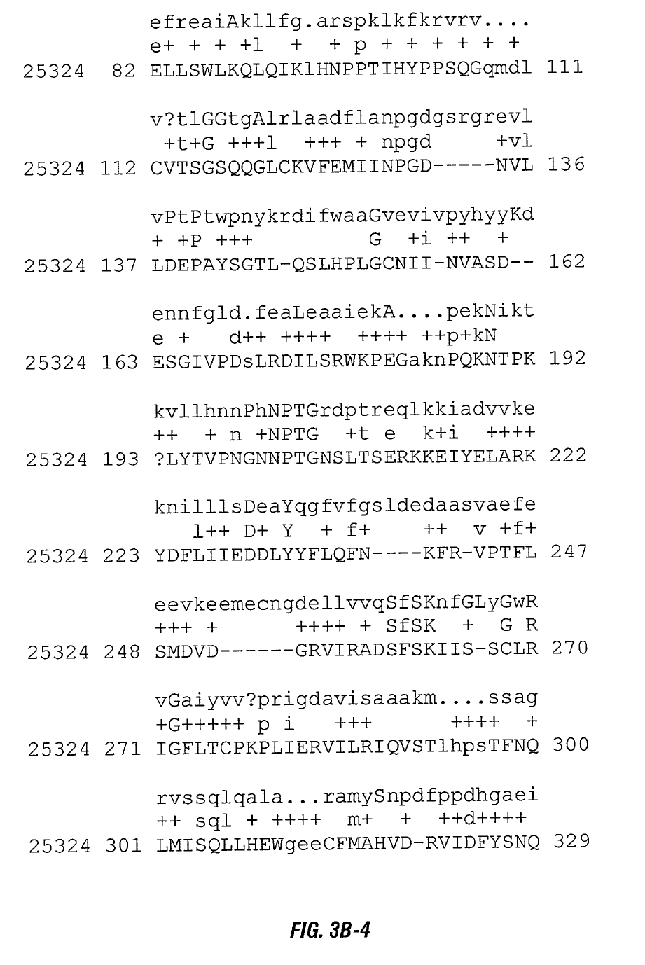

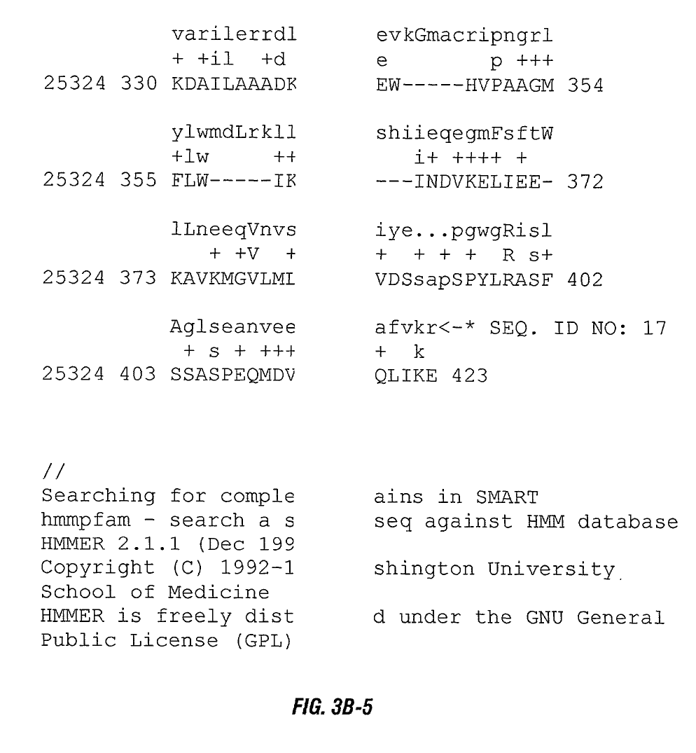

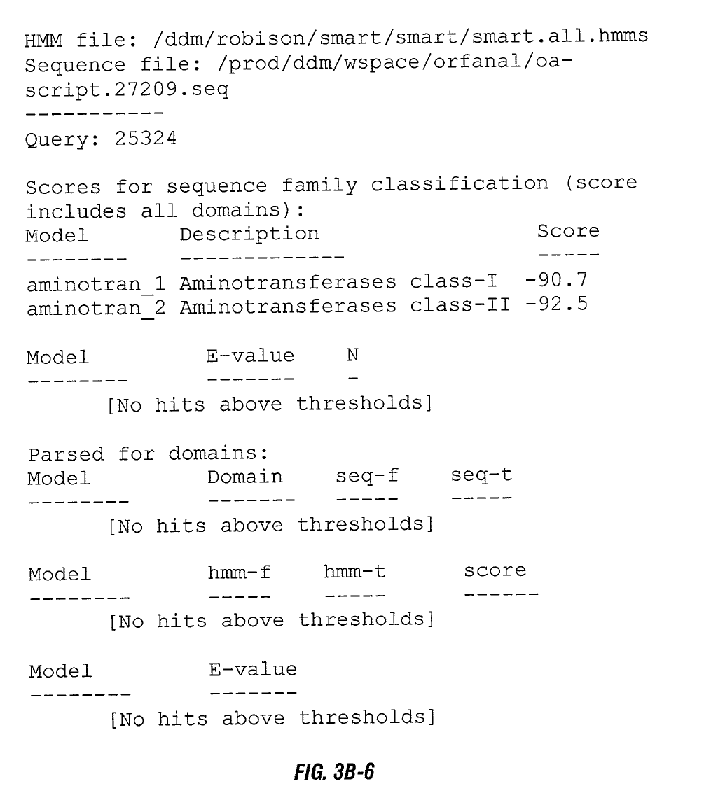

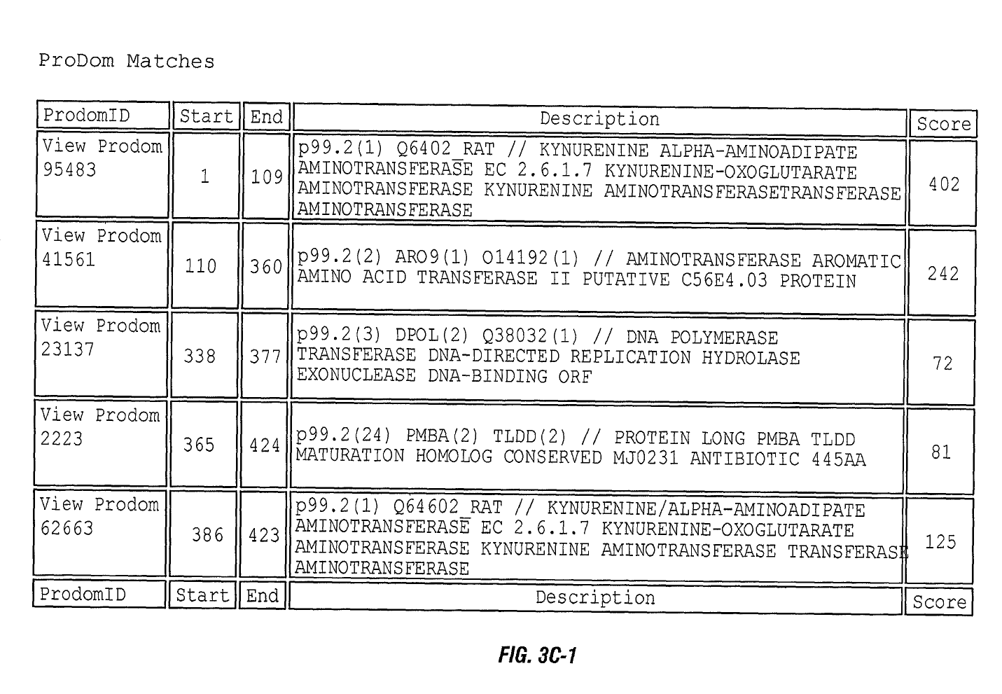

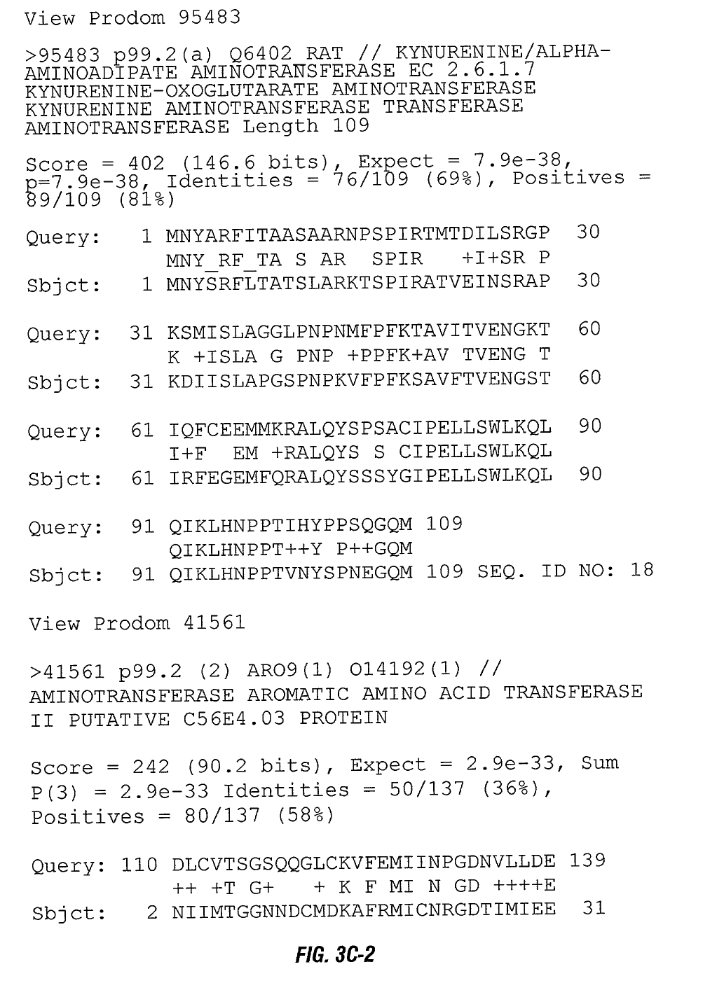

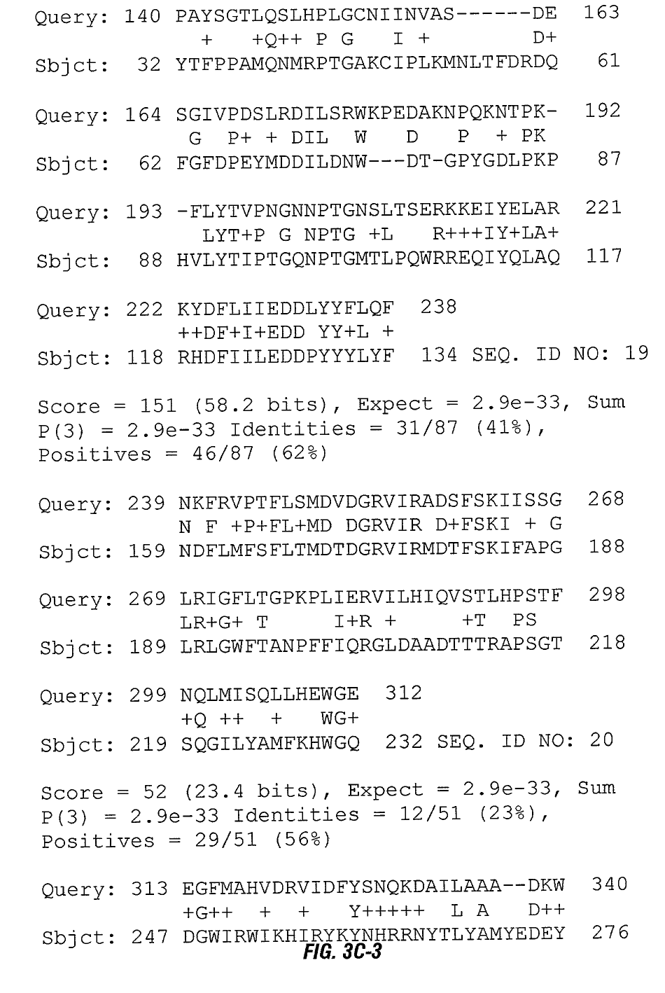

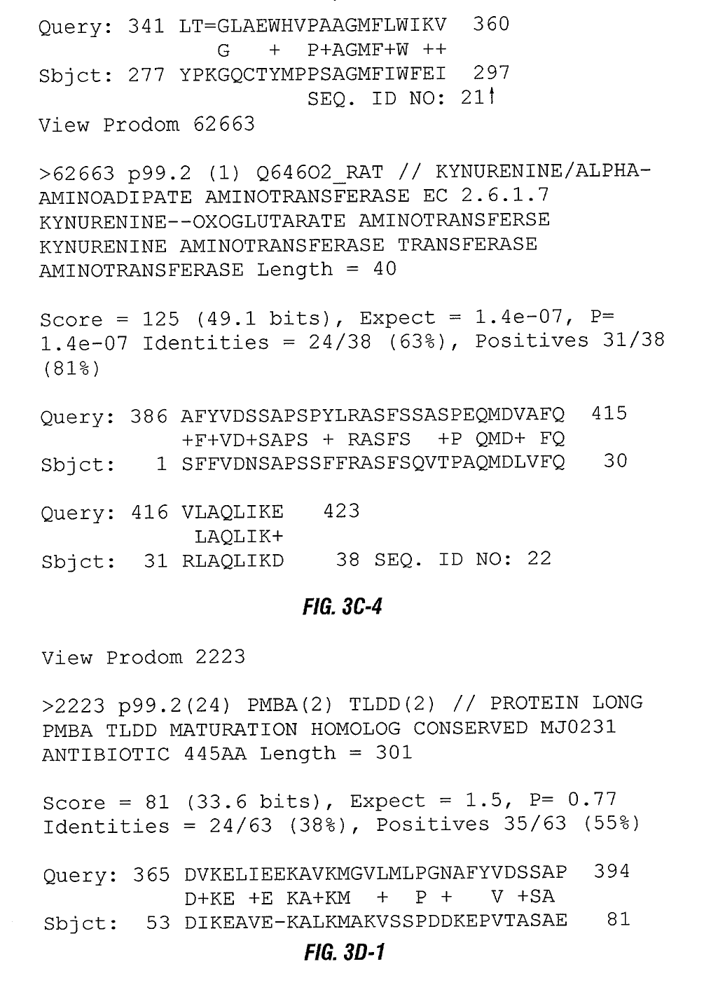

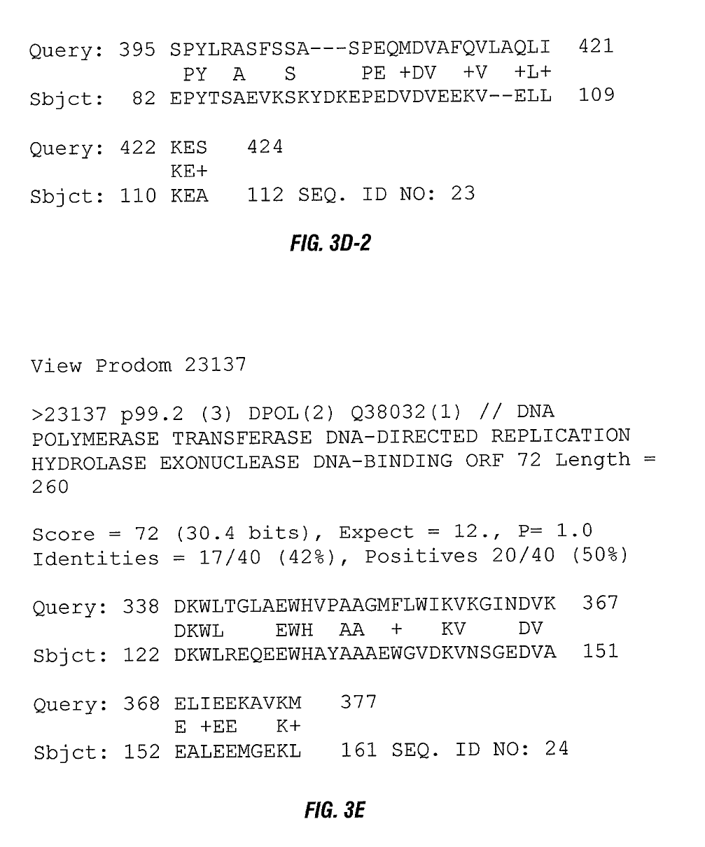

In another preferred embodiment, a member of this novel subfamily of transferase proteins has at least one transferase domain as shown in FIG. 3 which includes at least about 76-109 amino acid residues and has at least about 65-75% identity with the transferase domain of human transferase as shown in FIG. 3 (e.g., residues 1-109 of SEQ ID NO: 2). Preferably, the transferase domain as shown in FIG. 3 includes at least about 80-105 amino acid residues, or about 85-100 amino acid residues, or 80-90 amino acid residues, and has at least 70-80% identity, preferably about 80-85%, or more preferably about 85-95%, identity with the corresponding transferase domain shown in FIG. 3 of human transferase (e.g., residues 1-109 of SEQ ID NO: 2).

Accordingly, transferase proteins having at least 65-75% identity, preferably about 70-80%, more preferably about 80-85%, or most preferably about 85-95% identity with the corresponding transferase domain shown in FIG. 3 of human transferase are within the scope of the invention.

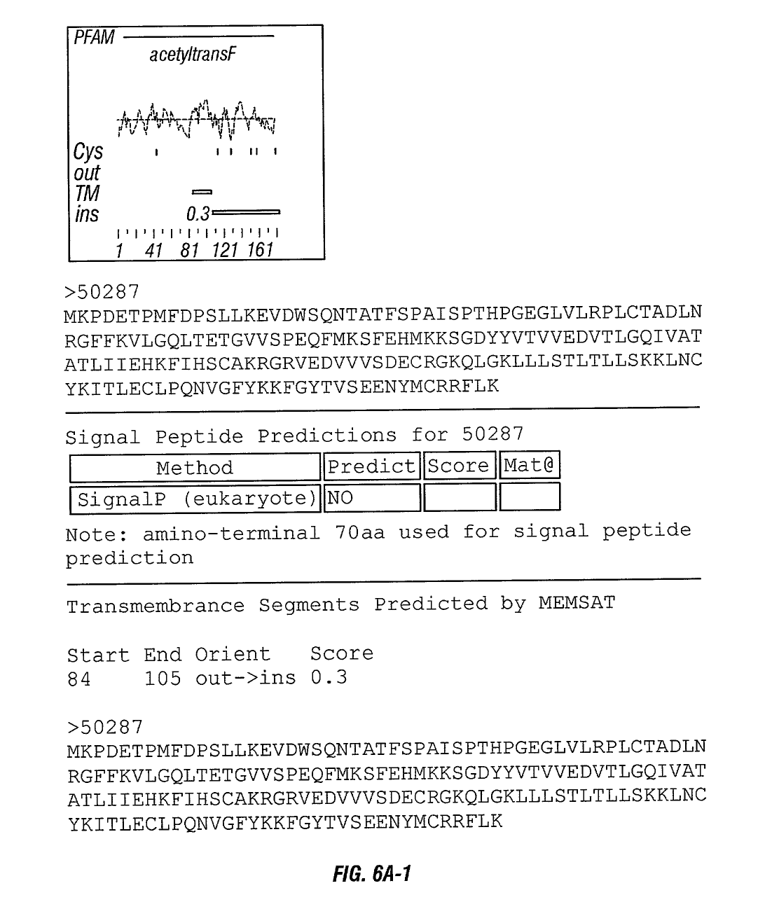

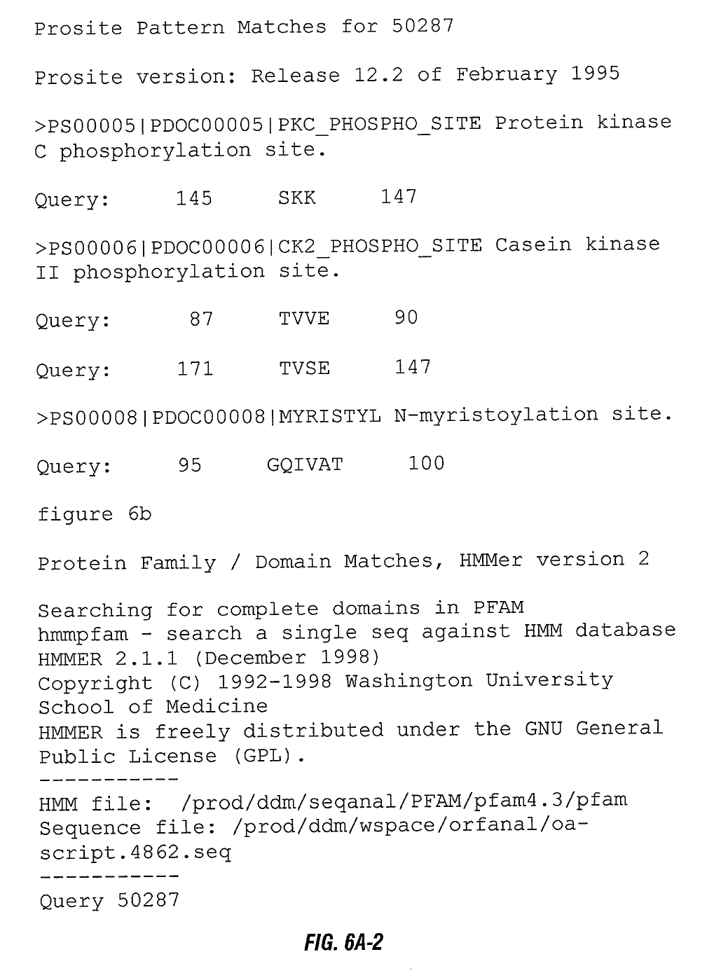

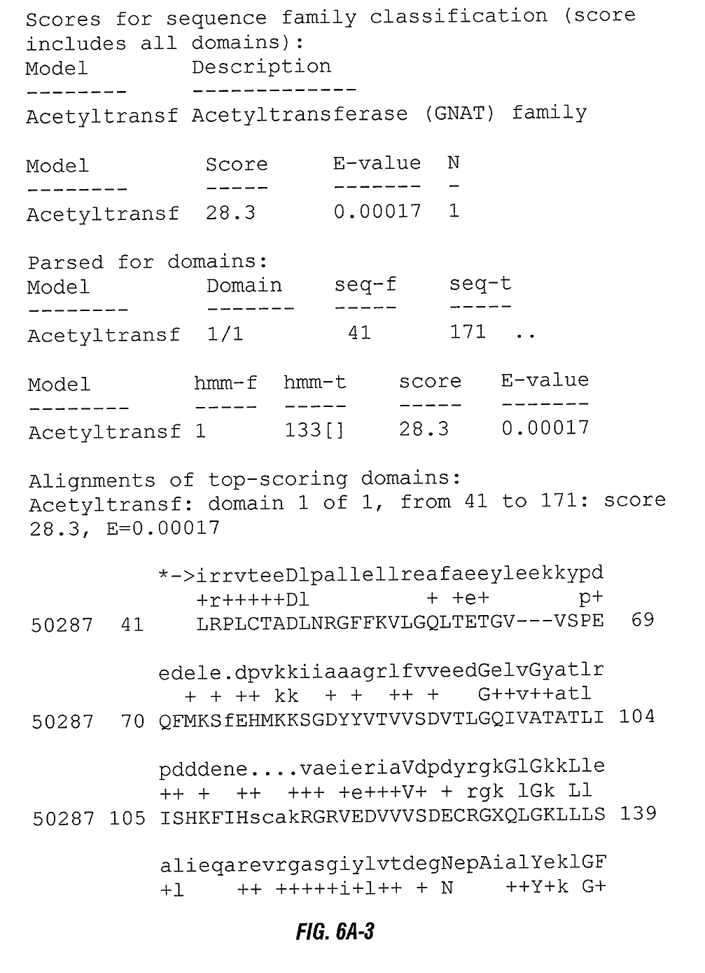

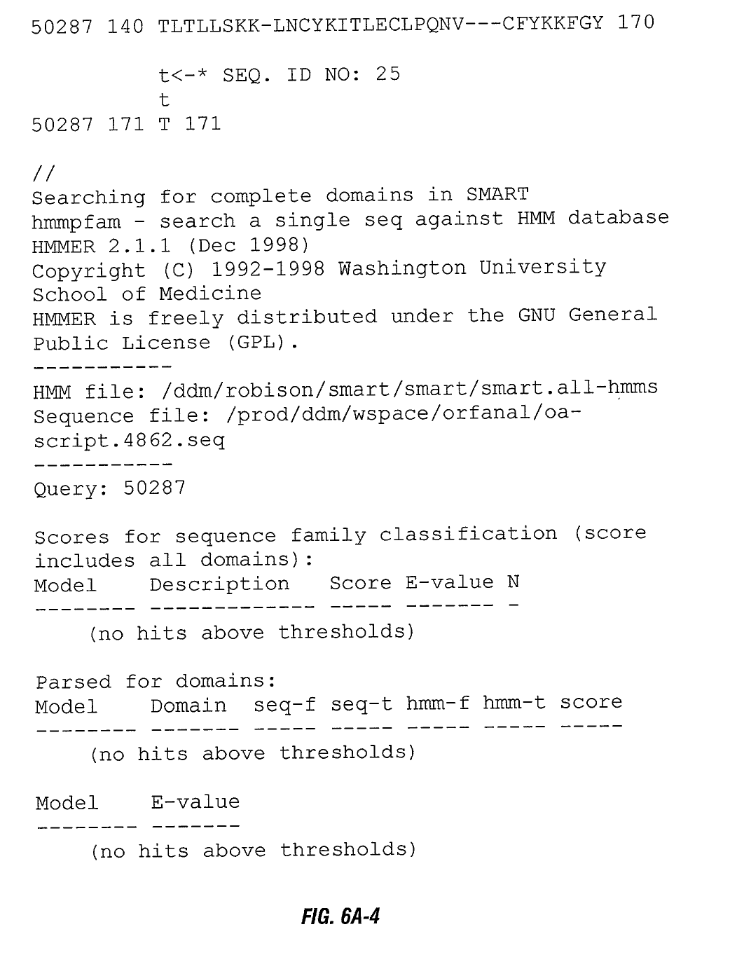

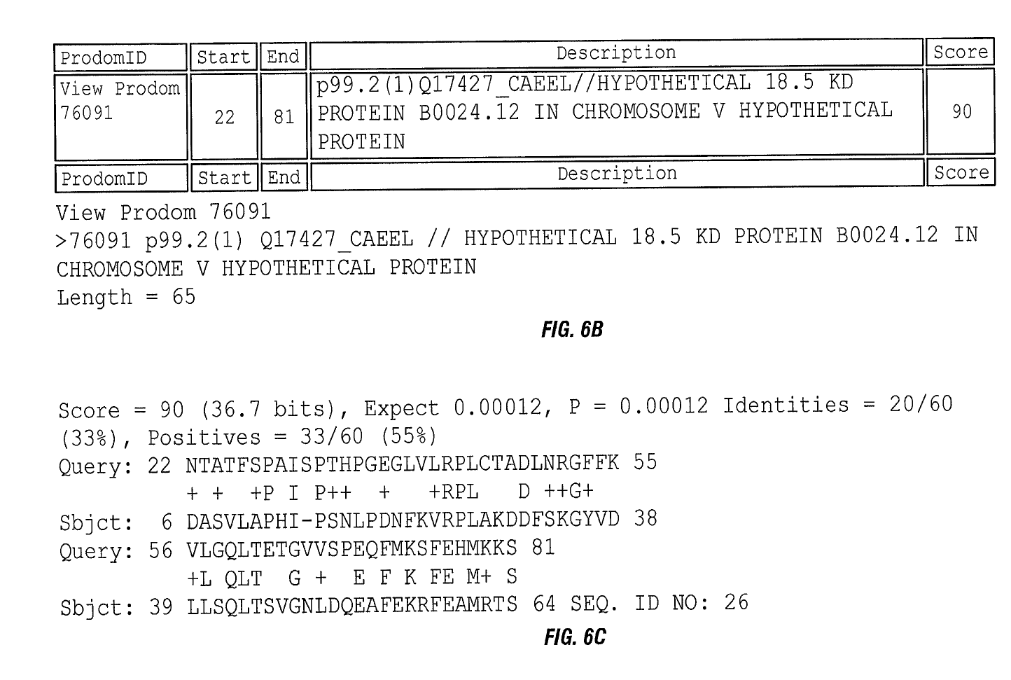

In another preferred embodiment, a member of this novel subfamily of transferase proteins has at least one transferase domain as shown in FIG. 6 which includes at least about 20-60 amino acid residues and has at least about 30-35% identity with the transferase domain of human transferase as shown in FIG. 6 (e.g., residues 6-64 of SEQ ID NO: 4). Preferably, the transferase domain as shown in FIG. 6 includes at least about 25-55 amino acid residues, or about 30-50 amino acid residues, or 35-45 amino acid residues, and has at least 35-55% identity, preferably about 55-65%, or more preferably about 65-75%, or even more preferably 75-85%, and most preferably 85-95% identity with the corresponding transferase domain shown in FIG. 6 of human transferase (e.g., residues 6-64 of SEQ ID NO: 4).

Accordingly, transferase proteins having at least 30-35% identity, preferably about 35-55%, more preferably about 55-65% or about 65-75%, or even more preferably 75-85% and most preferably 85-95% identity with the corresponding transferase domain shown in FIG. 6 of human transferase are within the scope of the invention.

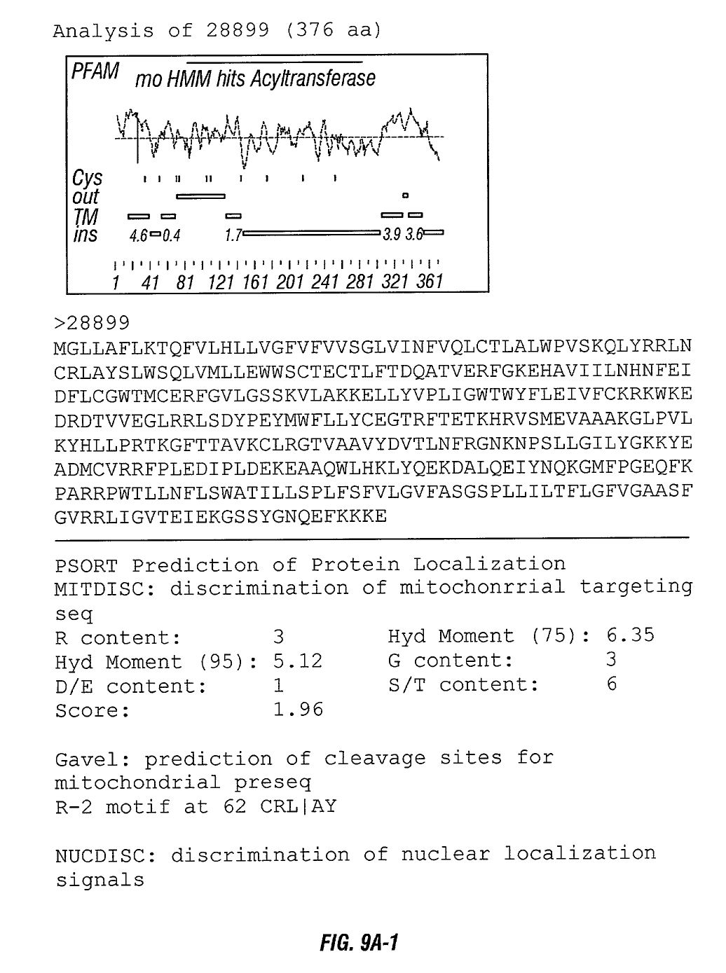

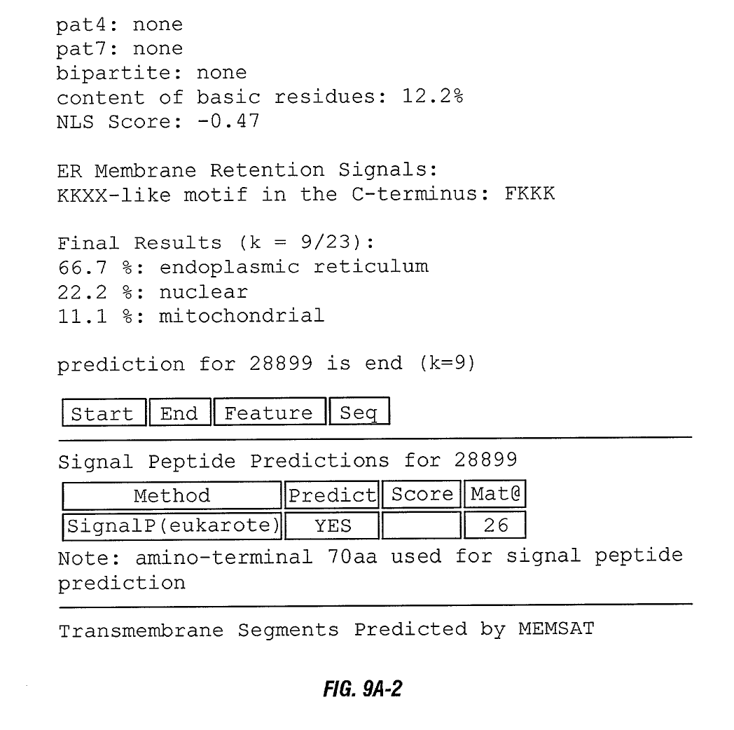

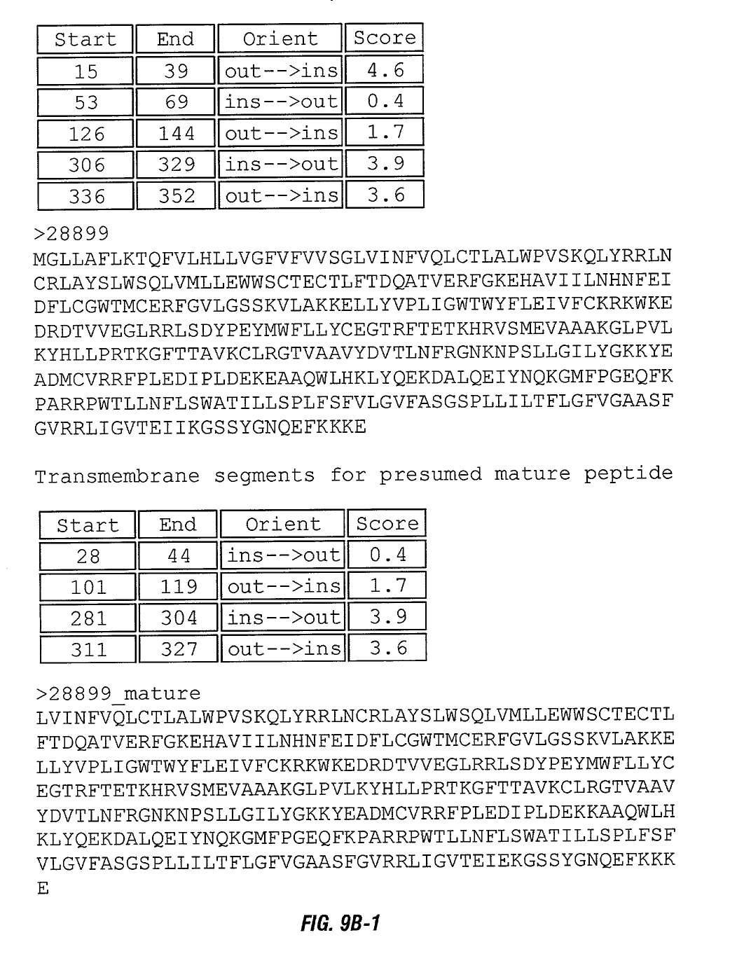

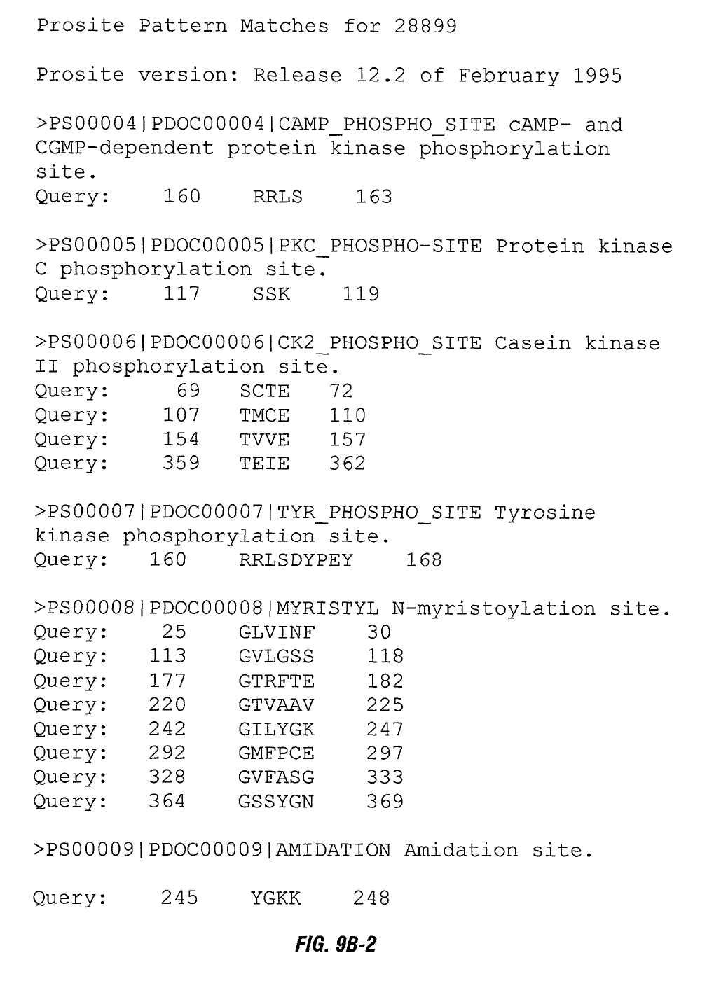

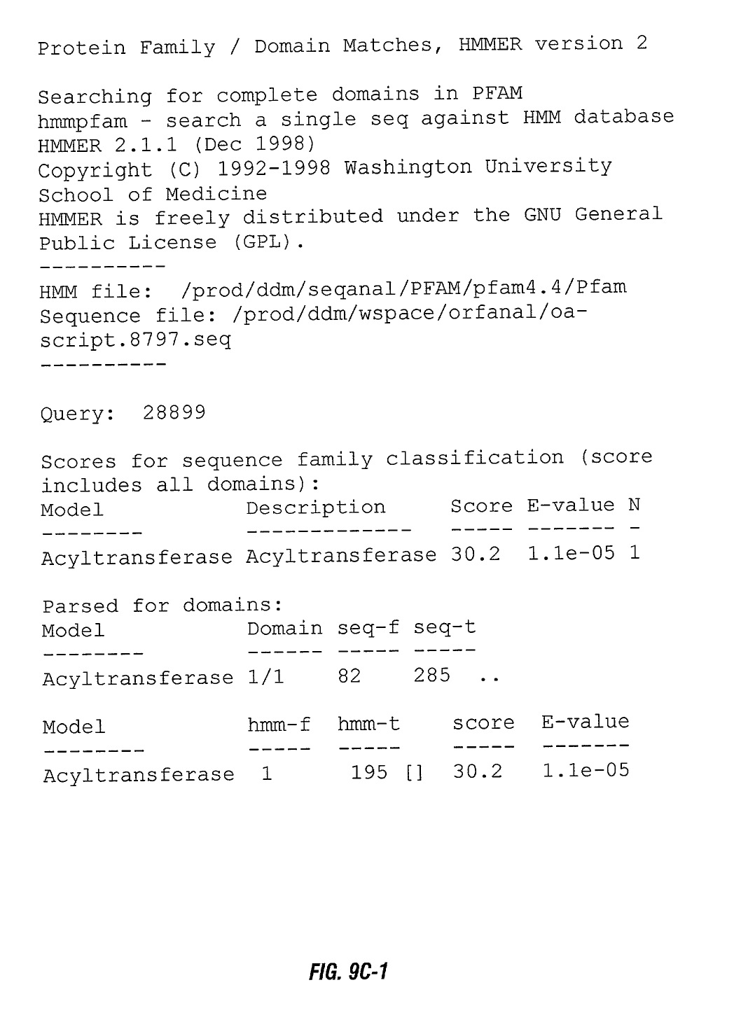

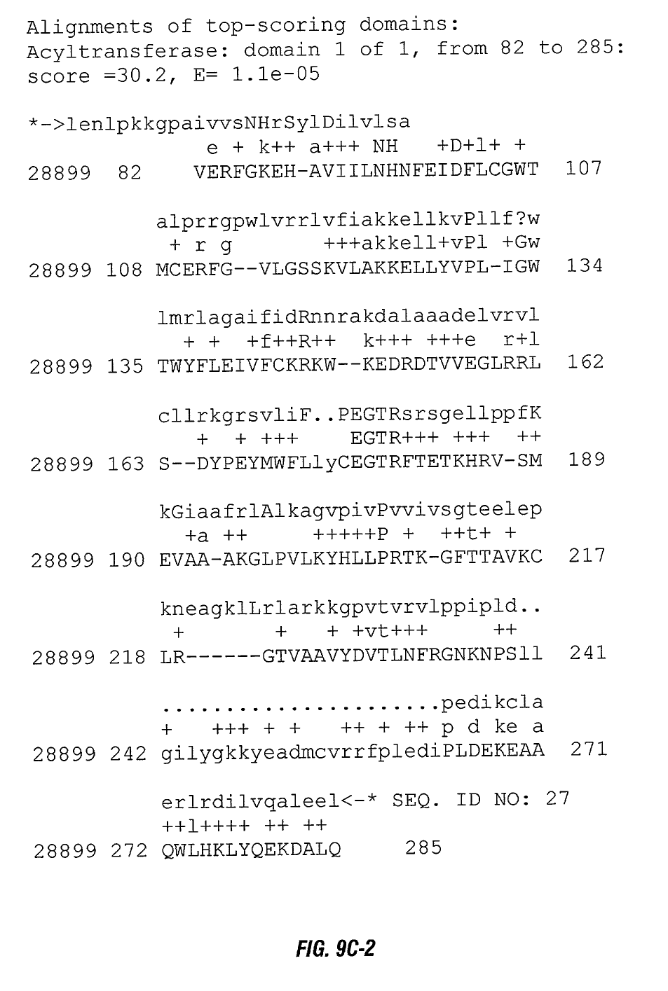

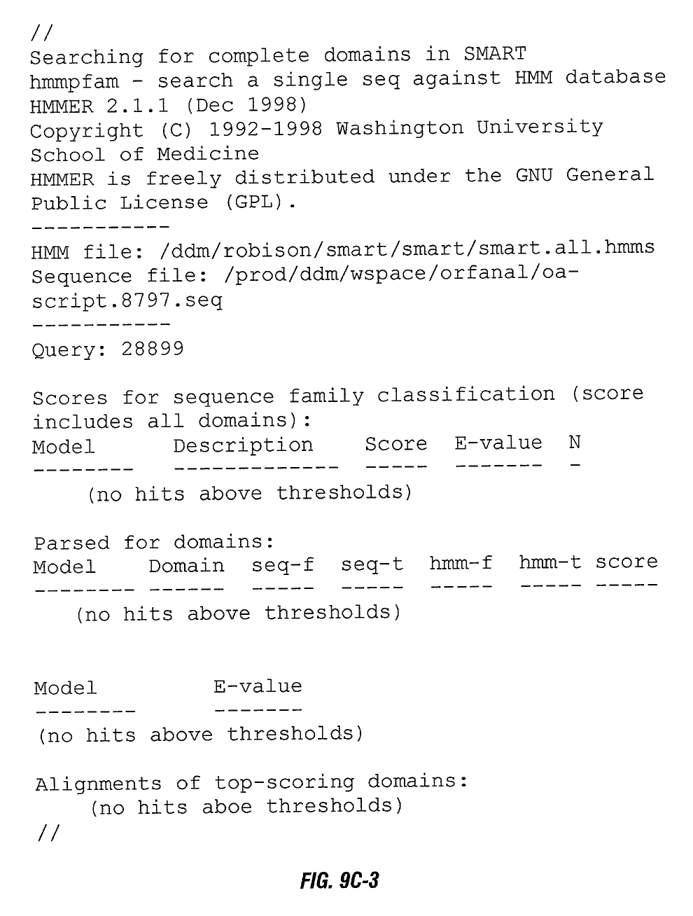

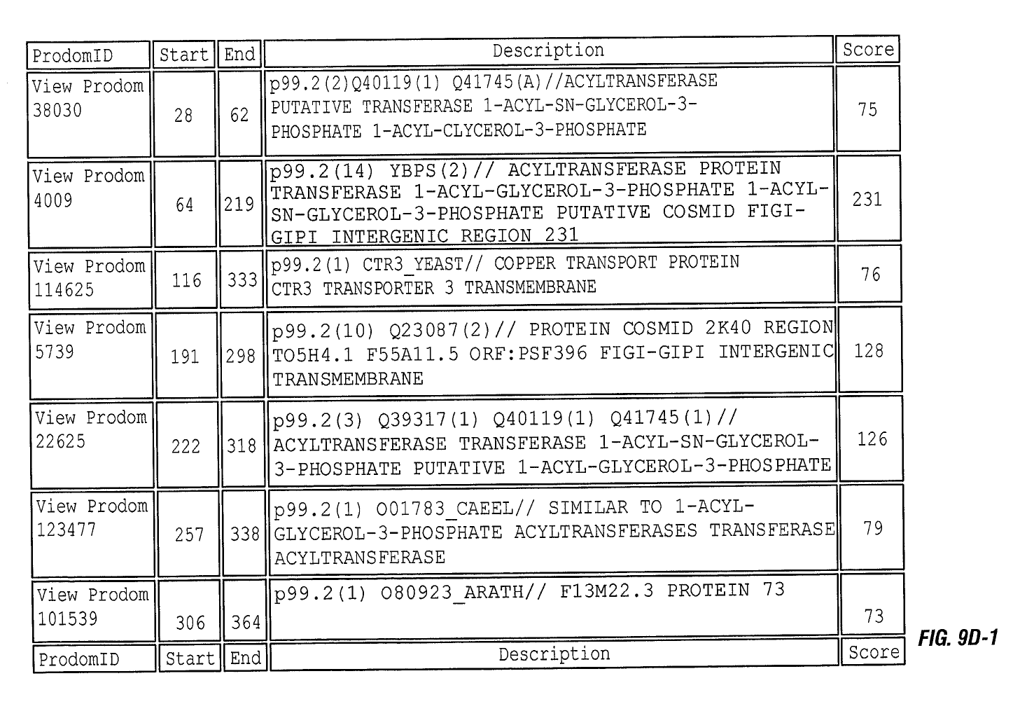

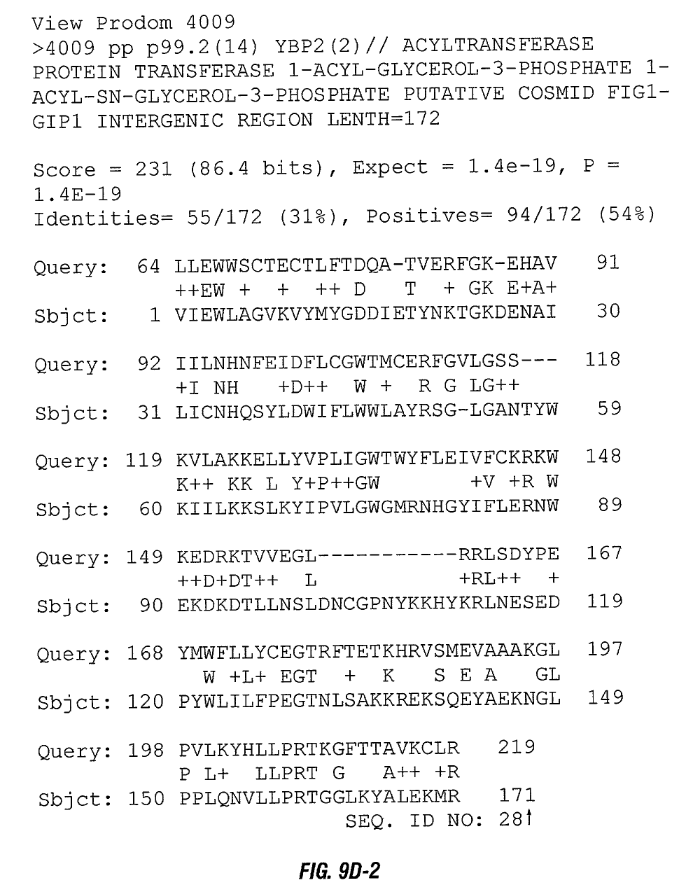

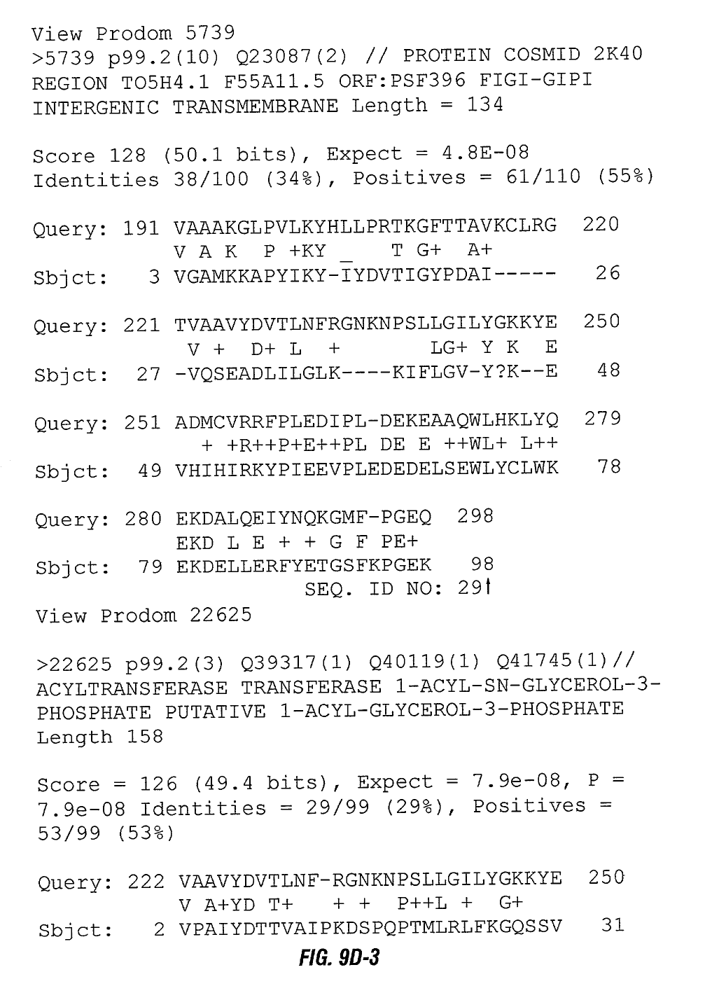

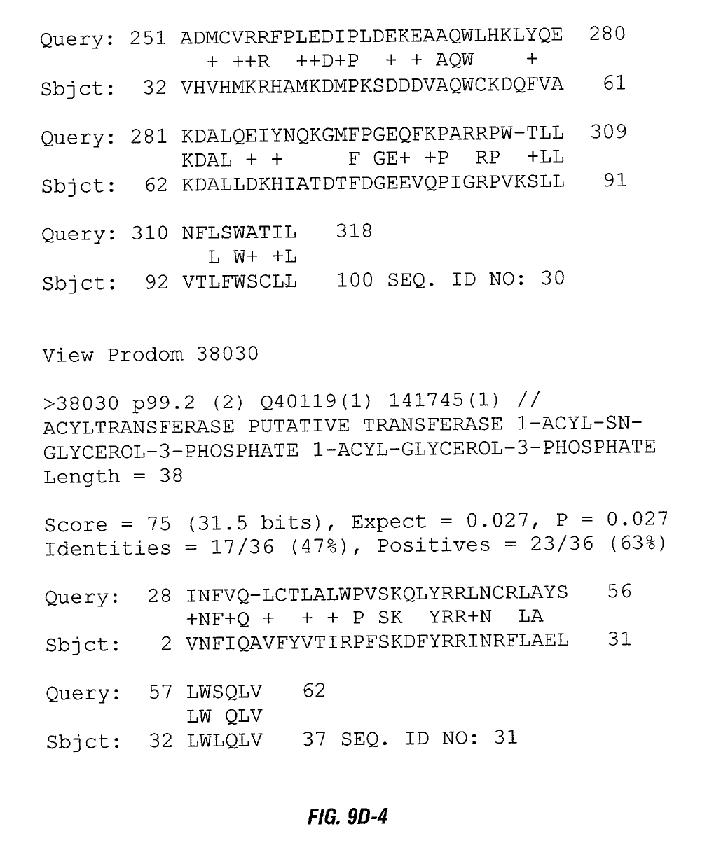

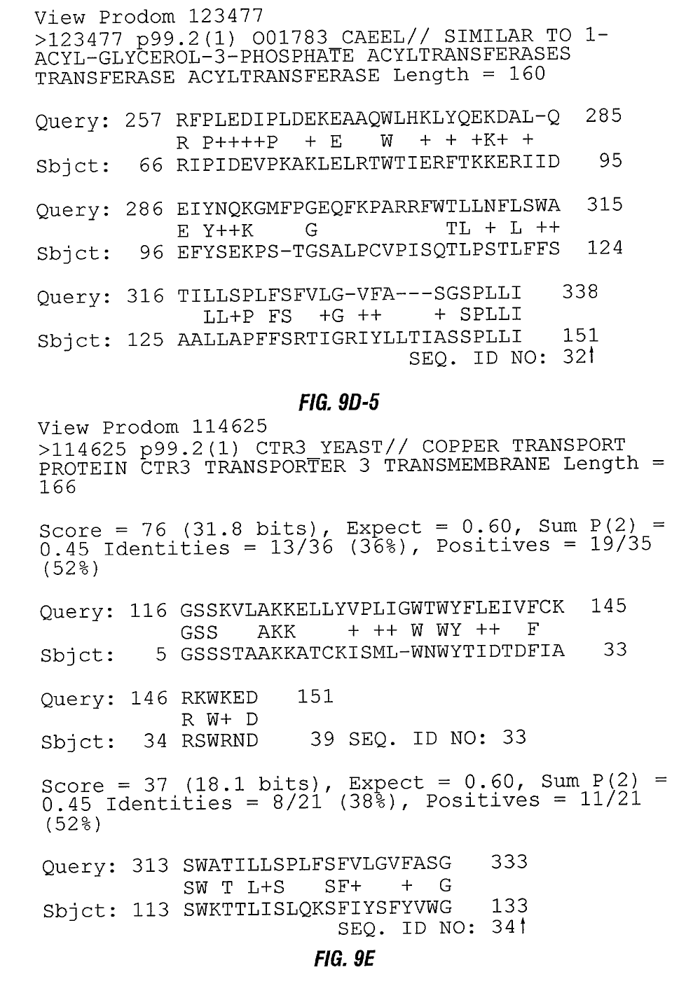

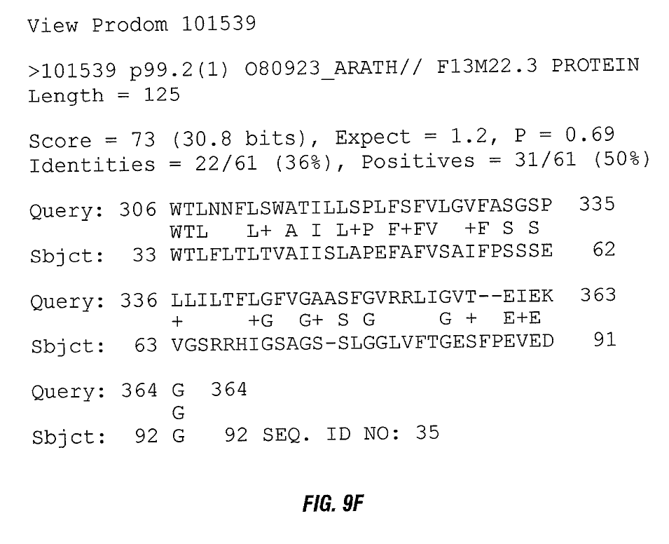

In another preferred embodiment, a member of this novel subfamily of transferase proteins has at least one transferase domain as shown in FIG. 9 which includes at least about 55-172 amino acid residues and has at least about 30-35% identity with the transferase domain of human transferase as shown in FIG. 9 (e.g., residues 1-171 of SEQ ID NO: 6). Preferably, the transferase domain as shown in FIG. 9 includes at least about 70-160 amino acid residues, or about 85-145 amino acid residues, or 90-130 amino acid residues, and has at least 35-55% identity, preferably about 55-65%, more preferably about 65-75%, or even more preferably 75-85% and most preferably 85-95% identity with the corresponding transferase domain shown in FIG. 9 of human transferase (e.g., residues 1-171 of SEQ ID NO: 6).

Accordingly, transferase proteins having at least 30-35% identity, preferably about 35-55%, more preferably about 55-65% or about 65-75%, or even more preferably 75-85% and most preferably 85-95% identity with the corresponding transferase domain shown in FIG. 9 of human transferase are within the scope of the invention.

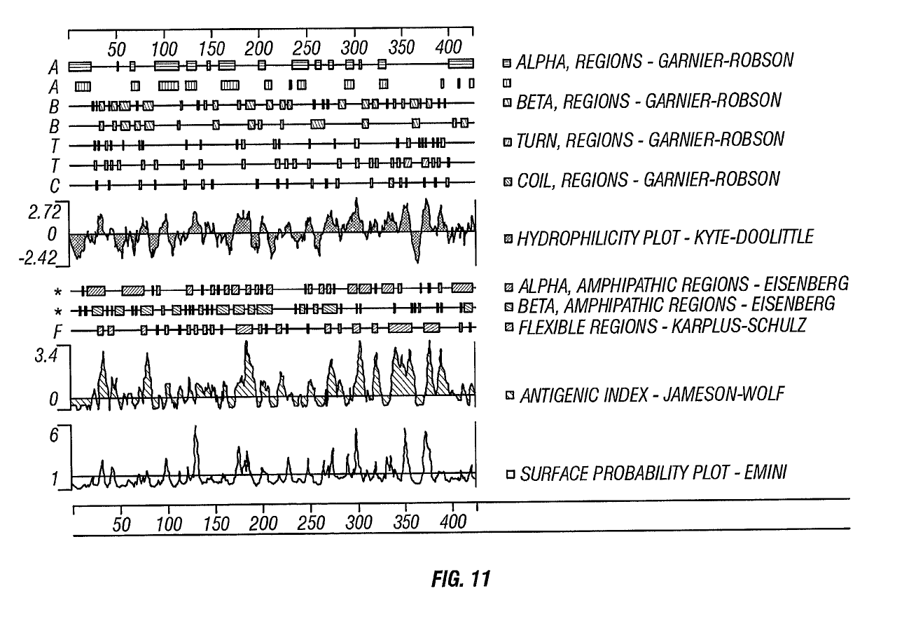

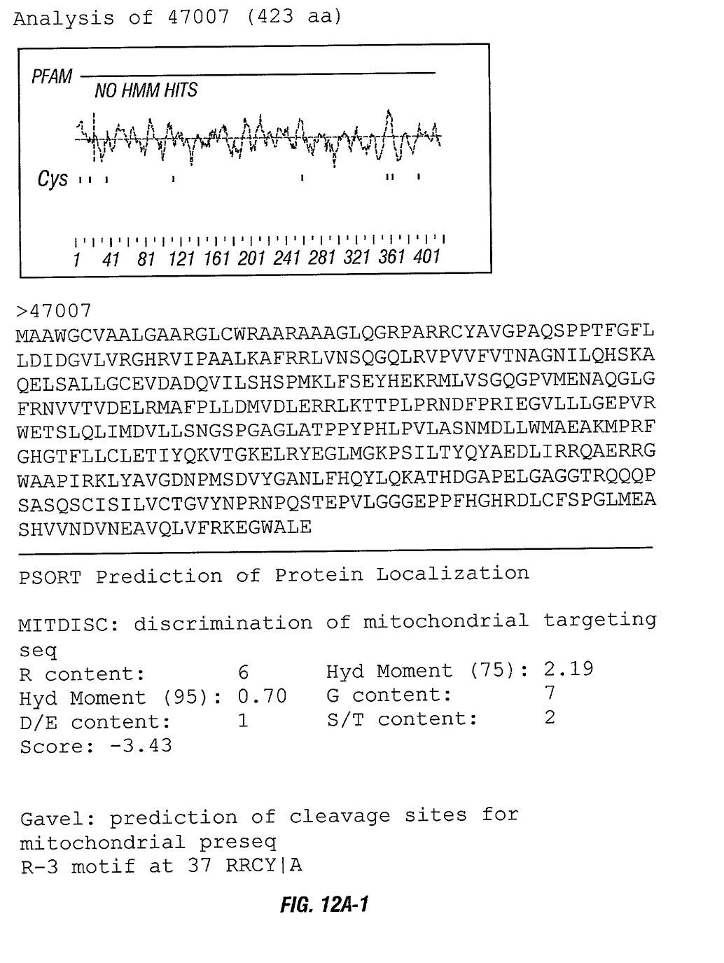

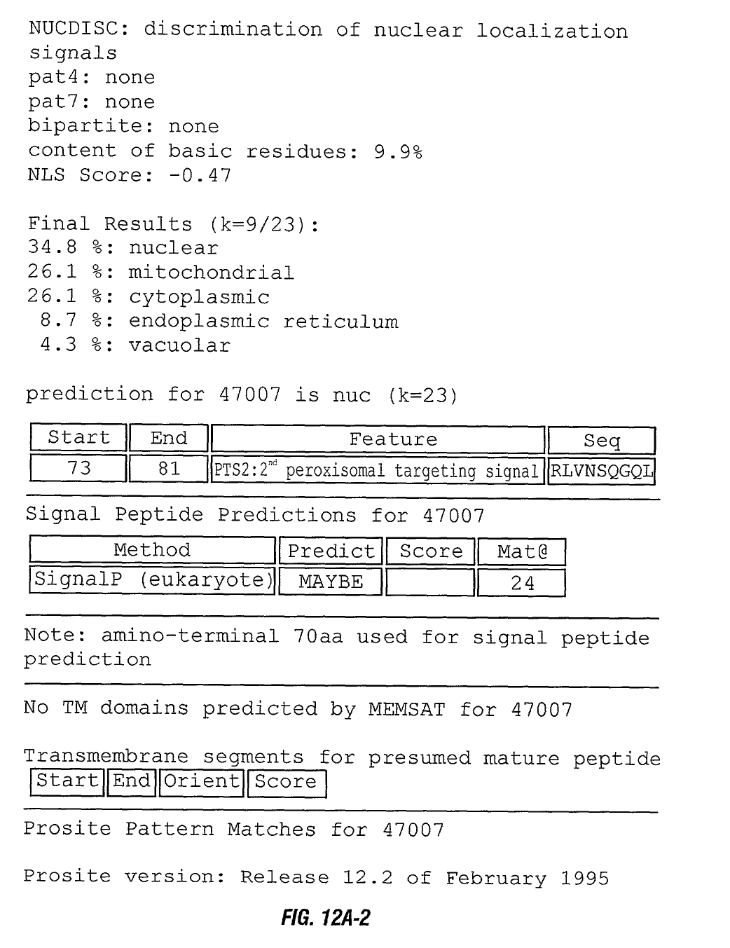

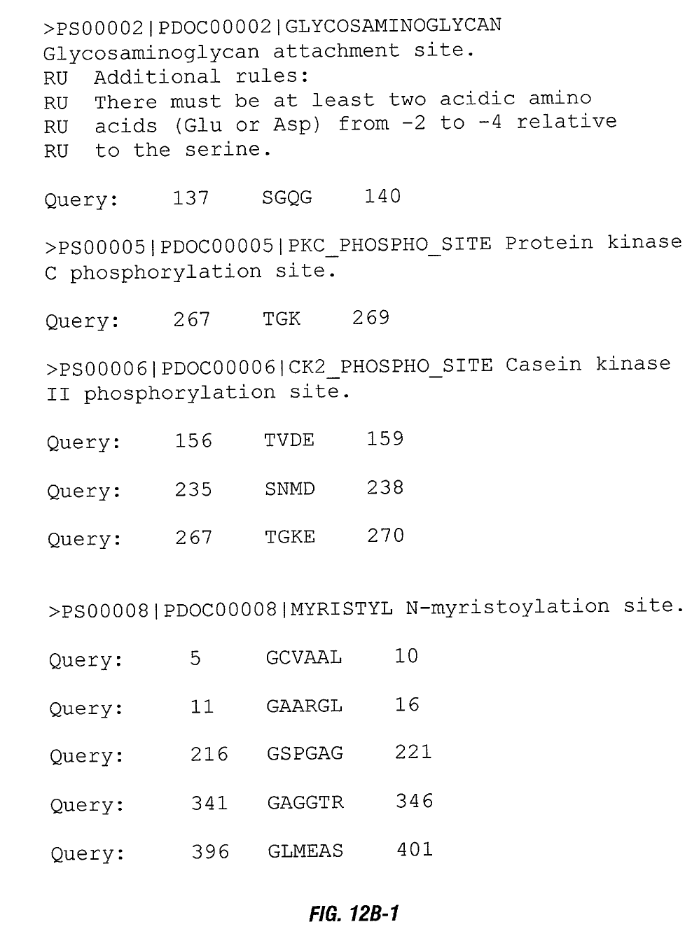

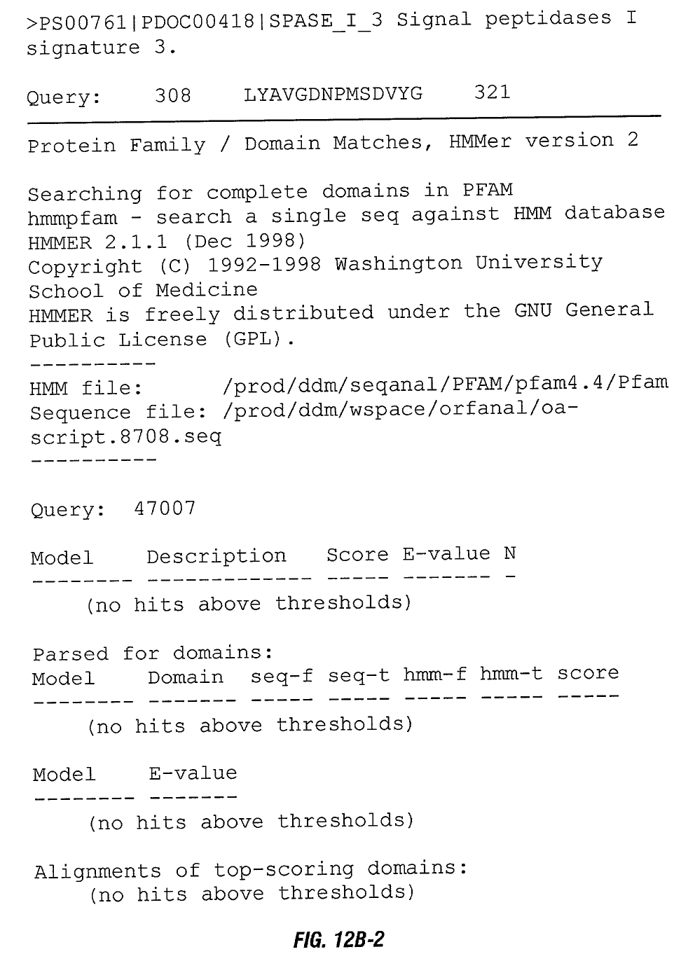

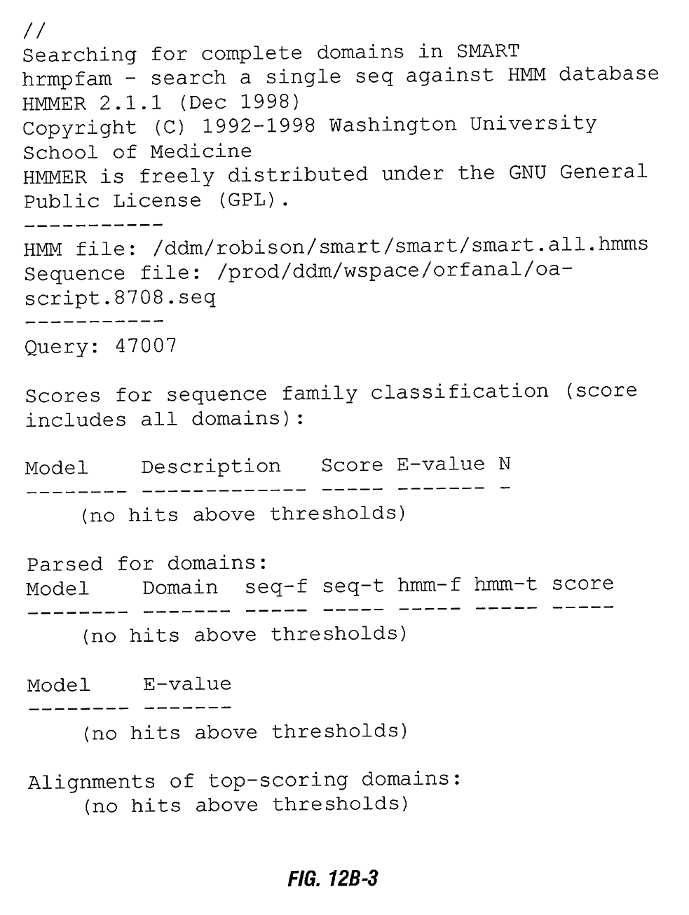

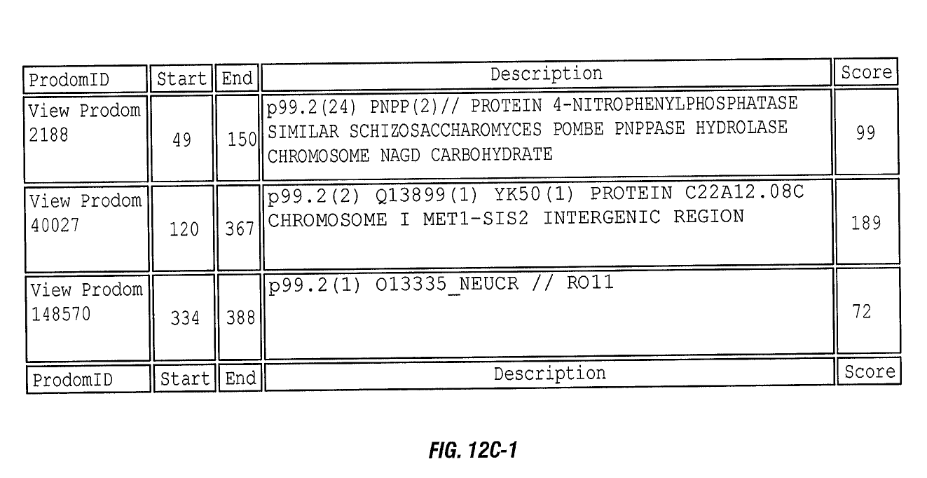

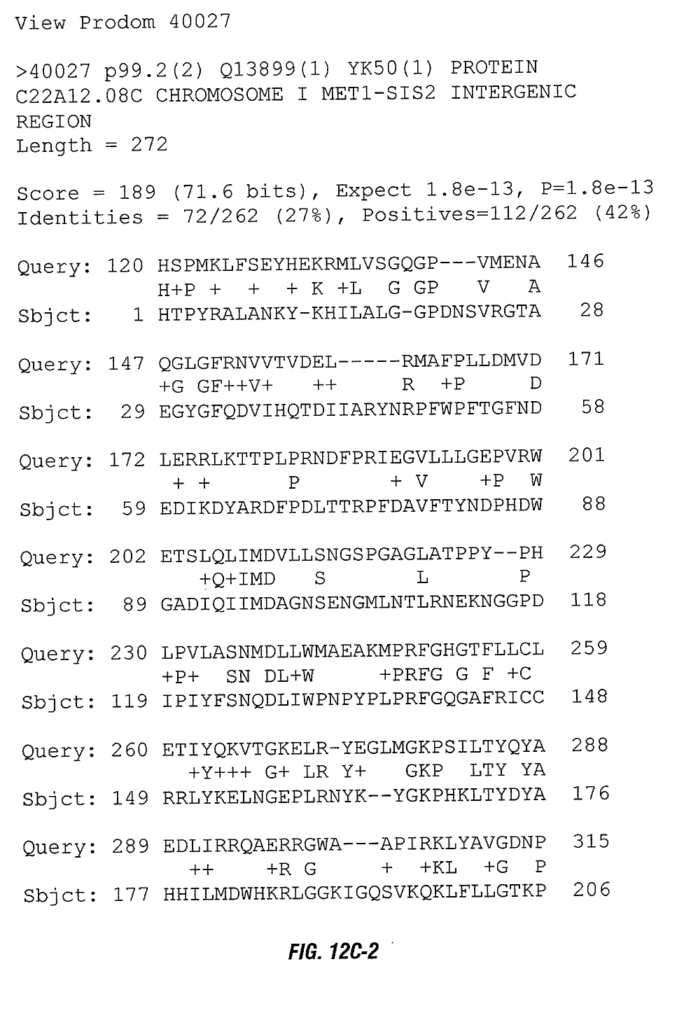

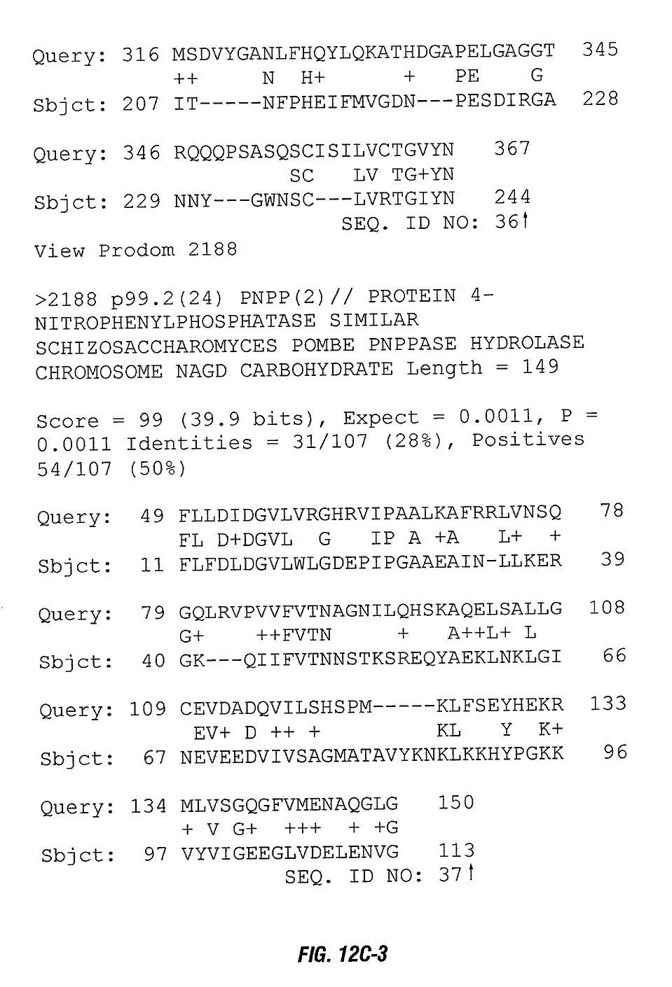

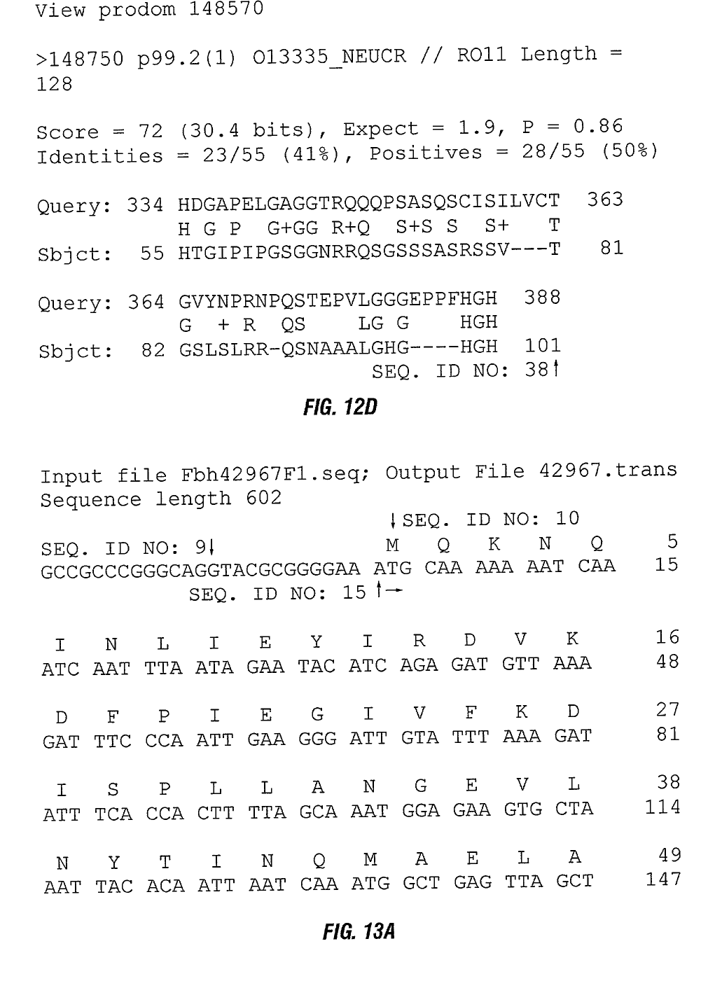

In another preferred embodiment, a member of this novel subfamily of transferase proteins has at least one transferase domain as shown in FIG. 12 which includes at least about 72-262 amino acid residues and has at least about 25-30% identity with the transferase domain of human transferase as shown in FIG. 12 (e.g., residues 1-244 of SEQ ID NO: 8). Preferably, the transferase domain as shown in FIG. 12 includes at least about 100-230 amino acid residues, or about 130-200 amino acid residues, or 160-170 amino acid residues, and has at least 30-45% identity, preferably about 45-60%, more preferably about 60-75%, or even more referably 75-85% and most preferably 85-95% identity with the corresponding transferase domain shown in FIG. 12 of human transferase (e.g., residues 1-244 of SEQ ID NO: 8).

Accordingly, transferase proteins having at least 25-30% identity, preferably about 30-45%, more preferably about 45-60% or about 60-75%, or even more preferably 75-85% and most preferably 85-95% identity with the corresponding transferase domain shown in FIG. 12 of human transferase are within the scope of the invention.

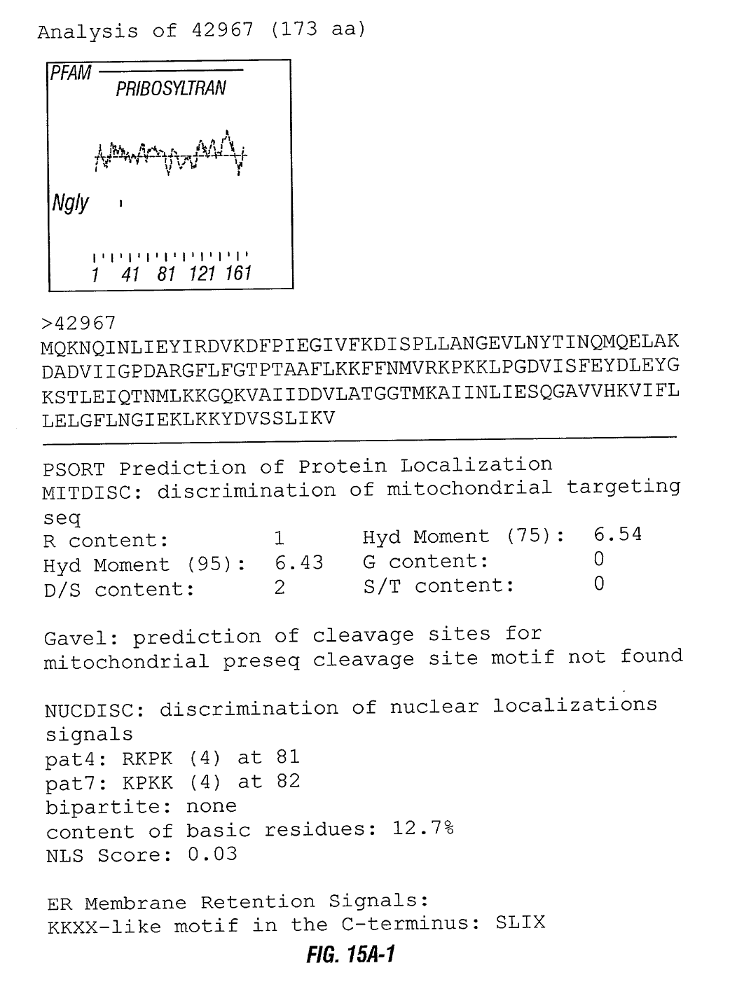

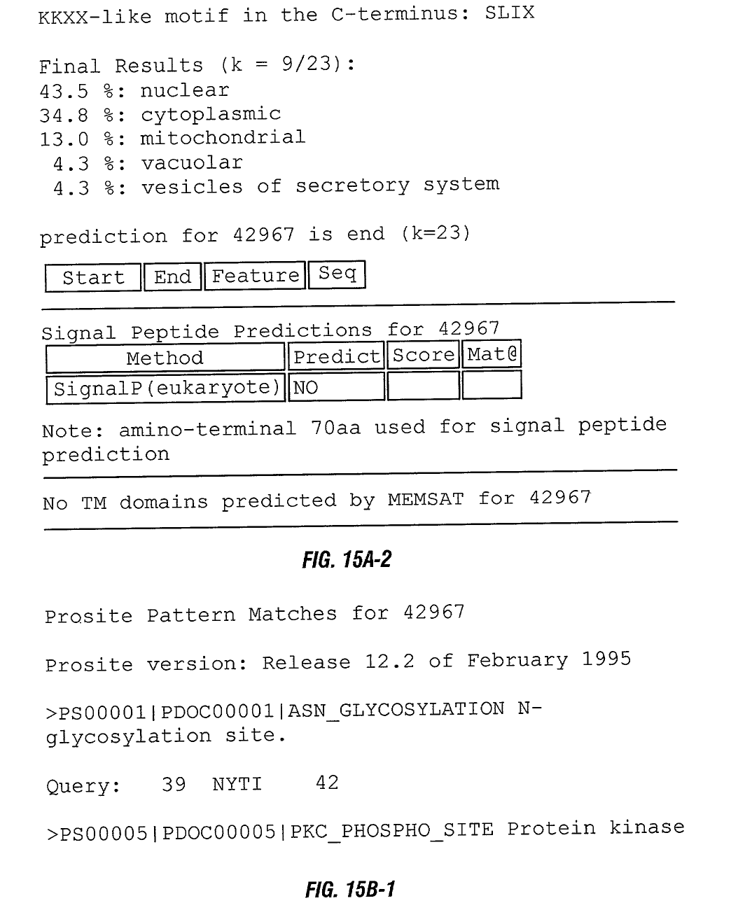

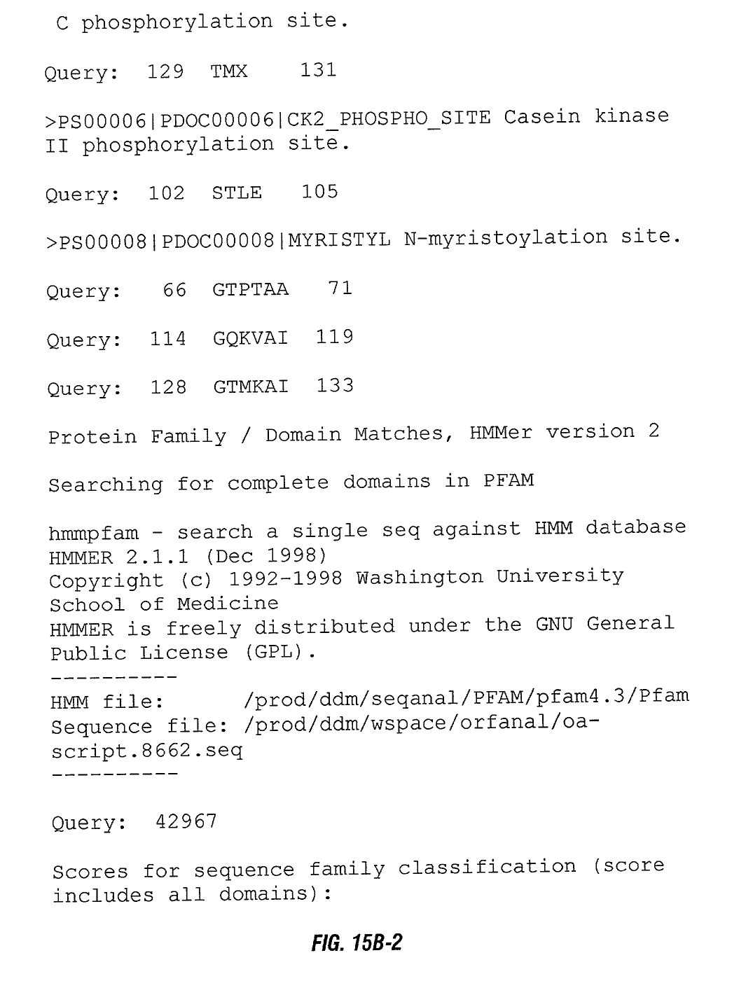

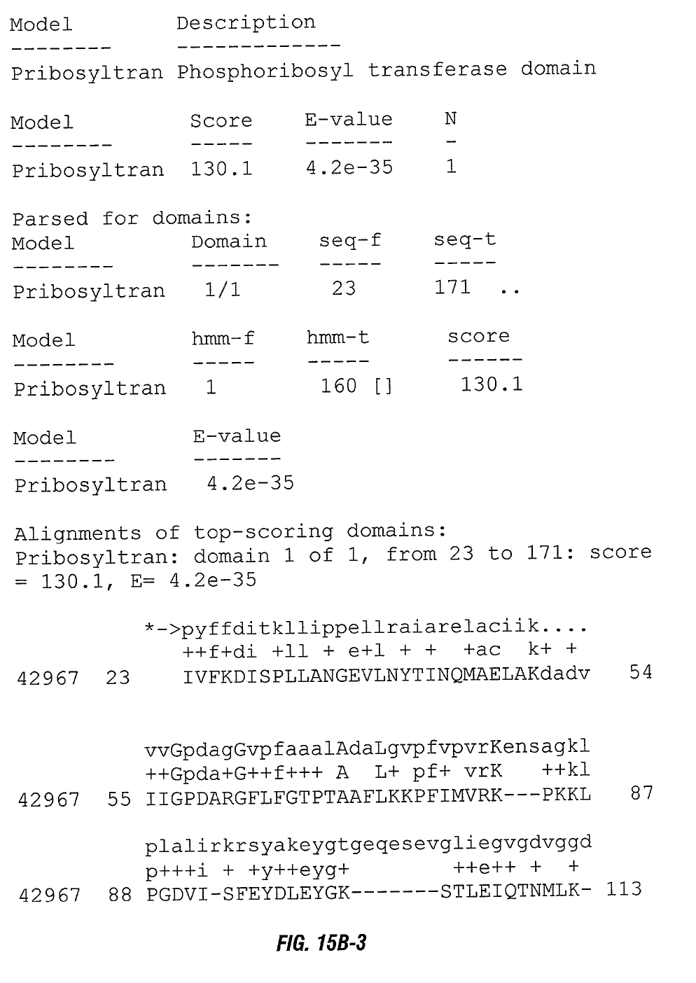

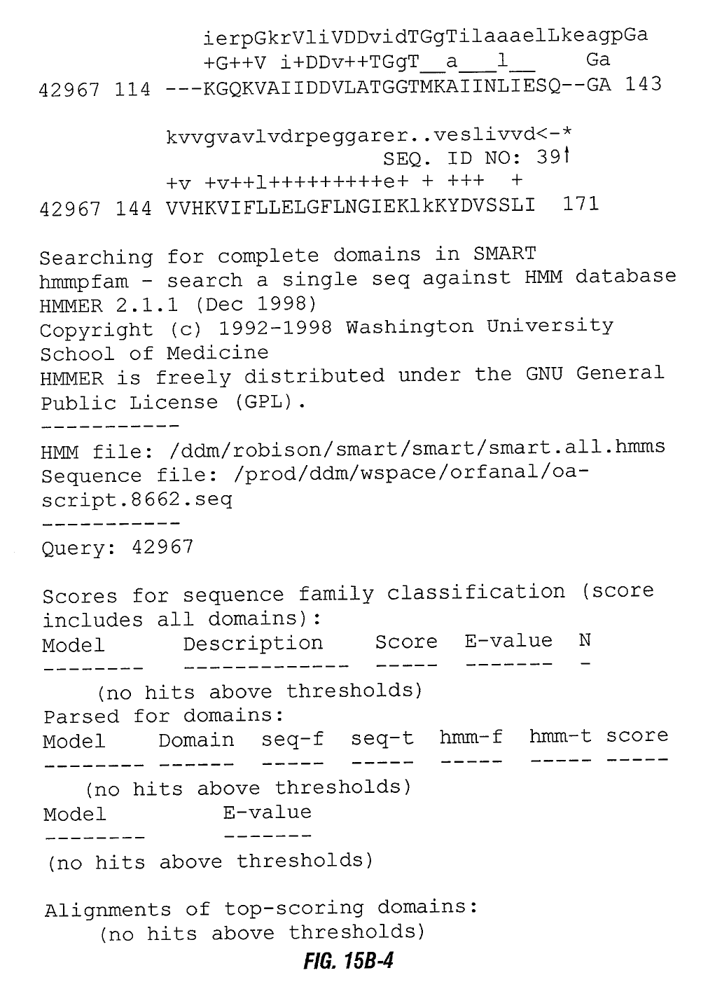

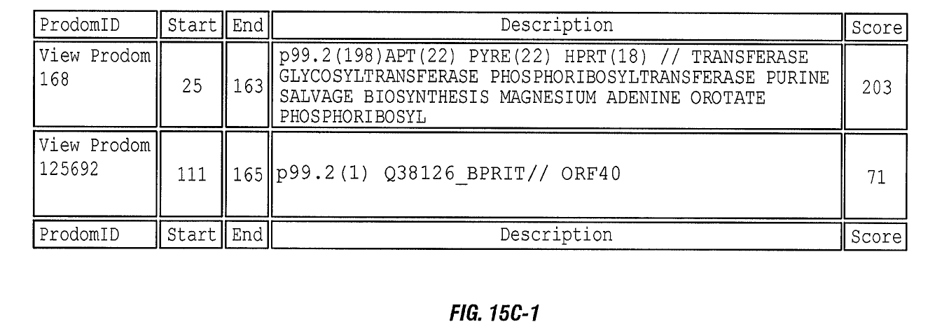

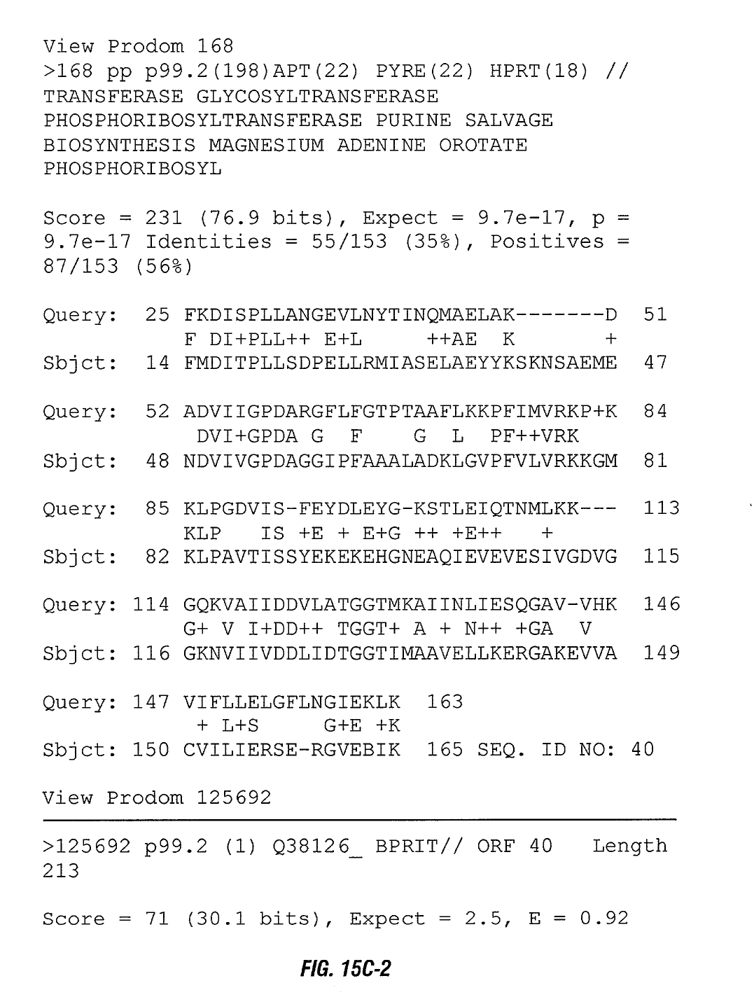

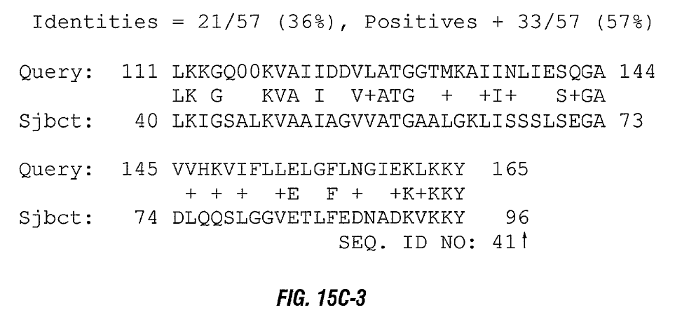

In another preferred embodiment, a member of this novel subfamily of transferase proteins has at least one transferase domain as shown in FIG. 15 which includes at least about 55-153 amino acid residues and has at least about 30-40% identity with the transferase domain of human transferase as shown in FIG. 15 (e.g., residues 14-165 of SEQ ID NO: 10). Preferably, the transferase domain as shown in FIG. 15 includes at least about 70-140 amino acid residues, or about 85-130 amino acid residues, or 95-115 amino acid residues, and has at least 40-55% identity, preferably about 55-70%, more preferably about 70-85%, and most preferably 85-95% identity with the corresponding transferase domain shown in FIG. 15 of human transferase (e.g., residues 14-165 of SEQ ID NO: 10).

Accordingly, transferase proteins having at least 30-40% identity, preferably about 4-055%, more preferably about 55-70% or about 70-85%, or most preferably 85-95% identity with the corresponding transferase domain shown in FIG. 15 of human transferase are within the scope of the invention.

Transferase family members can be identified based on the presence of at least one transferase domain as shown in FIGS. 3, 6, 9, 12, and 15 in the protein or the nucleic acid molecule encoding the protein.

As used herein, the term “domain as shown in FIG. 3” with regard to 25324 includes an aminotransferase domain having an amino acid sequence of about 270-320 or 390-440 amino acid residues and having a bit score for the alignment of the sequence to the transferase domain (HMM) of at least about 90. Preferably, an amino transferase domain includes at least about 280-300 or 390-420, more preferably about 290-300 or 415-420 amino acid residues, or 294-296 or 418-420 amino acid residues, and may have a bit score for the alignment of the sequence to the transferase domain (HMM) of at least about 90, 95, 100, 110, 120 or greater. The aminotransferase domains (HMM) as shown in FIG. 3 has been assigned the PFAM Accession PF00155 (class I) or PF00222 (class II) (http://genome.wustl.edu/Pfam/.html).

As used herein, the term “aminotransferase domain” with regard to 25324 includes an amino acid sequence which is conserved in aminotransferases. Preferably, the aminotransferase domain includes one of the following amino acid consensus sequences [GS]-[LIVMFYTAC][GSTA]-K-x(2)-[GSALVN]-[LIVMFA]-x-[GNAR]-x-R-[LIVMA]-[GA] or T-[LIVMFYW][STAG]-K-[SAG]-[LIVMFYWR]-[SAG]-x(2)-[SAG], wherein K is the pyridoxal-P attachment site.

The 25324 protein includes the following domains: four predicted protein kinase C phosphorylation sites (PS00005) located at about amino acids 170-172, 190-192, and 210-212 of SEQ ID NO: 2; two predicted casein kinase II phosphorylation sites (PS00006) located at about amino 21-24 and 170-173 of SEQ ID NO: 2; and four predicted N-myristoylation sites (PS00008) located at about amino acids 116-121, 144-149, 200-205, and 268-273 of SEQ ID NO: 2.

As used herein, the term “domain as shown in FIG. 6” with regard to 50287 includes a protein domain having an amino acid sequence of about 80-150 amino acid residues and having a bit score for the alignment of the sequence to the acetyltransferase domain (HMM) of at least about 25. Preferably, an acetyl transferase domain includes at least about 80-140, more preferably about 125-135 amino acid residues, or 129-131 amino acid residues, and has a bit score for the alignment of the sequence to the transferase domain (HMM) of at least about 25, 30, 35, 40, 50, 60 or greater. The acetyltransferase domain (HMM) as shown in FIG. 6 has been assigned the PFAM Accession PF00583 (http://genome.wustl.edu/Pfam/.html).

The 50287 protein includes the following domains: one predicted protein kinase C phosphorylation site (PS00005) located at about amino acids 145-147 of SEQ ID NO: 4; two predicted casein kinase II phosphorylation sites (PS00006) located at about amino 87-90 and 171-174 of SEQ ID NO: 4; and one predicted N-myristoylation site (PS00008) located at about amino acids 95-100 of SEQ ID NO: 4.

In one embodiment, a 50287 protein includes at least one transmembrane domain. As used herein, the term “transmembrane domain” includes an amino acid sequence of about 15 amino acid residues in length that spans a phospholipid membrane. More preferably, a transmembrane domain includes about at least 18, 20, 22, 24, 25, 30, 35 or 40 amino acid residues and spans a phospholipid membrane. Transmembrane domains are rich in hydrophobic residues, and typically have an a-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, http://pfam.wustl.edu/cgi-bin/getdesc?name=7tm-1, and Zagotta W.N. et al., (1996) Annual Rev. Neuronsci. 19:235-63, the contents of which are incorporated herein by reference.

In a preferred embodiment, a 50287 polypeptide or protein has at least one transmembrane domain or a region which includes at least 18, 20, 22, 24, 25, 30, 35 or 40 amino acid residues and has at least about 60%, 70% 80% 90% 95%, 99%, or 100% homology with a “transmembrane domain,” e.g., at least one transmembrane domain of human 50287 (e.g., amino acid residues 84-105 of SEQ ID NO: 4).

In another embodiment, a 50287 protein includes at least one “non-transmembrane domain.” As used herein, “non-transmembrane domains” are domains that reside outside of the membrane. When referring to plasma membranes, non-transmembrane domains include extracellular domains (i.e., outside of the cell) and intracellular domains (i.e., within the cell). When referring to membrane-bound proteins found in intracellular organelles (e.g., mitochondria, endoplasmic reticulum, peroxisomes and microsomes), non-transmembrane domains include those domains of the protein that reside in the cytosol (i.e., the cytoplasm), the lumen of the organelle, or the matrix or the intermembrane space (the latter two relate specifically to mitochondria organelles). The C-terminal amino acid residue of a nontransmembrane domain is adjacent to an N-terminal amino acid residue of a transmembrane domain in a naturally-occurring 50287, or 50287-like protein.

In a preferred embodiment, a 50287 polypeptide or protein has a “non-transmembrane domain” or a region which includes at least about 25-200, preferably about 50-100, more preferably about 70-90, and even more preferably about 75-85 amino acid residues, and has at least about 60%, 70% 80% 90% 95%, 99% or 100% homology with a “non-transmembrane domain”, e.g., a non-transmembrane domain of human 50287 (e.g., residues 1-83 and 106-184 of SEQ ID NO: 4). Preferably, a non-transmembrane domain is capable of catalytic activity (e.g., catalyzing a transferase reaction).

A non-transmembrane domain located at the N-terminus of a 50287 protein or polypeptide is referred to herein as an “N-terminal non-transmembrane domain.” As used herein, an “N-terminal non-transmembrane domain” includes an amino acid sequence having about 25-200, preferably about 50-100, more preferably about 70-90, and even more preferably about 75-85 amino acid residues in length and is located outside the boundaries of a membrane. For example, an N-terminal non-transmembrane domain is located at about amino acid residues 1-83 of SEQ ID NO: 4.

Similarly, a non-transmembrane domain located at the C-terminus of a 50287 protein or polypeptide is referred to herein as a “C-terminal non-transmembrane domain.” As used herein, an “C-terminal non-transmembrane domain” includes an amino acid sequence having about 25200, preferably about 50-100, more preferably about 70-90, and even more preferably about 75-85 amino acid residues in length and is located outside the boundaries of a membrane. For example, a C-terminal non-transmembrane domain is located at about amino acid residues 106184 of SEQ ID NO: 4.

As used herein, the term “domain as shown in FIG. 9” with regard to 28899 includes a protein domain having an amino acid sequence of about 180-220 amino acid residues and having a bit score for the alignment of the sequence to the transferase domain (HMM) of at least about 25. Preferably, an acyltransferase domain includes at least about 180-210, more preferably about 195-205 amino acid residues, 202-204 amino acid residues, and has a bit score for the alignment of the sequence to the transferase domain (HMM) of at least about 30, 35, 40, 50, 60 or greater. The acyltransferase domain (HMM) as shown in FIG. 9 has been assigned the PFAM Accession PF01553 (httD://genome.wustl.edu/Pfam/.html).

The 28899 protein includes the following domains: one cAMP- and cGMP-dependent protein kinase phosphorylation site (PS00004) located at about amino acids 160-163 of SEQ ID NO: 6; one predicted protein kinase C phosphorylation site (PS00005) located at about amino acids 117-119 of SEQ ID NO: 6; four predicted casein kinase II phosphorylation sites (PS00006) located at about amino 69-72, 107-110, 154-157 and 359-362 of SEQ ID NO: 6; one predicted tyrosine kinase phosphorylation site (PS00007) located at about amino acids 160-168 of SEQ ID NO: 6; eight predicted N-myristoylation sites (PS00008) located at about amino acids 25-30, 113-118, 177-182, 220-225, 242-247, 292-297, 328-333, and 364-369 of SEQ ID NO: 6; one predicted amidation site (PS00009) located at about amino acids 245-248 of SEQ ID NO: 6.

A 28899 molecule can further include a signal sequence. As used herein, a “signal sequence” refers to a peptide of about 10-80 amino acid residues in length which occurs at the N-terminus of secretory and integral membrane proteins and which contains a majority of hydrophobic amino acid residues. For example, a signal sequence contains at least about 10-50 amino acid residues, preferably about 20-30 amino acid residues, more preferably about 25 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer. For example, in one embodiment, a 28899 protein contains a signal sequence of about amino acids 1-25 of SEQ ID NO: 6. The “signal sequence” is cleaved during processing of the mature protein.

The mature 28899 protein form is approximately 351 amino acid residues in length (from about amino acid 26 to amino acid 376 of SEQ ID NO: 6) In a preferred embodiment, a 28899 polypeptide or protein has at least one transmembrane domain or a region which includes at least 18, 20, 22, 24, 25, 30, 35 or 40 amino acid residues and has at least about 60%, 70% 80% 90% 95%, 99%, or 100% homology with a “transmembrane domain,” e.g., at least one transmembrane domain of human 28899 (e.g., amino acid residues 53-69, 126-144, 306-329, or 336-352 of SEQ ID NO: 6).

In another embodiment, a 28899 protein includes at least one “non-transmembrane domain.” As used herein, “non-transmembrane domains” are domains that reside outside of the membrane. When referring to plasma membranes, non-transmembrane domains include extracellular domains (i.e., outside of the cell) and intracellular domains (i.e., within the cell). When referring to membrane-bound proteins found in intracellular organelles (e.g., mitochondria, endoplasmic reticulum, peroxisomes and microsomes), non-transmembrane domains include those domains of the protein that reside in the cytosol (i.e., the cytoplasm), the lumen of the organelle, or the matrix or the intermnembrane space (the latter two relate specifically to mitochondria organdlles). The C-terminal amino acid residue of a nontransmembrane domain is adjacent to an N-terminal amino acid residue of a transmembrane domain in a naturally-occurring 28899 , or 28899-like protein.

In a preferred embodiment, a 28899 polypeptide or protein has a “non-transmembrane domain” or a region which includes at least about 5-200, preferably about 5-180, more preferably about 5-170, and even more preferably about 5-160 amino acid residues, and has at least about 60%, 70% 80% 90% 95%, 99% or 100% homology with a “non-transmembrane domain”, e.g., a non-transmembrane domain of human 28899 (e.g., residues 26-52, 70-125, 145-305, 330-337, and 353-376 of SEQ ID NO: 6). Preferably, a non-transmembrane domain is capable of catalytic activity (e.g., catalyzing a transferase reaction).

A non-transmembrane domain located at the N-terminus of a 28899 protein or polypeptide is referred to herein as an “N-terminal non-transmembrane domain.” As used herein, an “N-terminal non-transmembrane domain” includes an amino acid sequence having about 5-100, preferably about 10-60, more preferably about 10-50, and even more preferably about 20-40 amino acid residues in length and is located outside the boundaries of a membrane. For example, an N-terminal non-transmembrane domain is located at about amino acid residues 1-52 or 26-52, in the mature protein, of SEQ ID NO: 6.

Similarly, a non-transmembrane domain located at the C-terminus of a 28899 protein or polypeptide is referred to herein as a “C-terminal non-transmembrane domain.” As used herein, an “C-terminal non-transmembrane domain” includes an amino acid sequence having about 1100, preferably about 20-75, more preferably about 20-50, and even more preferably about 2030 amino acid residues in length and is located outside the boundaries of a membrane. For example, a C-terminal non-transmembrane domain is located at about amino acid residues 353376 of SEQ ID NO: 6.

The 47007 protein includes the following domains: one glycosaminoglycan attachment site (PS00002) located at about amino acids 137-140 of SEQ ID NO: 8; one predicted protein kinase C phosphorylation site (PS00005) located at about amino acids 267-269 of SEQ ID NO: 8; three predicted casein kinase II phosphorylation sites (PS00006) located at about amino 156-159, 235-238 and 267-270 of SEQ ID NO: 8; five predicted N-myristoylation sites (PS00008) located at about amino acids 25-10, 11-16, 216-221, 341-346, and 396-401 of SEQ ID NO: 8; one predicted signal peptidase I signature site (PS00761) located at about amino acids 308-321 of SEQ ID NO: 8.

As used herein, the term “domain as shown in FIG. 15” with regard to 42967 includes a phosphoribosyl transferase domain having an amino acid sequence of about 130-170 amino acid residues and having a bit score for the alignment of the sequence to the transferase domain (HMM) of at least about 125. Preferably, a phosphoribosyl transferase domain includes at least about 140-160, more preferably about 145-150 amino acid residues, 147-149 amino acid residues, and has a bit score for the alignment of the sequence to the transferase domain (HMM) of at least about 130, 135, 140, 150, 160 or greater. The phosphoribosyl transferase domain (HMM) as shown in FIG. 15 has been assigned the PFAM Accession PF00156 (htto://genome.wustl.edu/Pfam/.html).

As used herein, the term “phosphoribosyl transferase domain” includes an amino acid sequence which is conserved in phosphoribosyl transferases. Preferably, the phosphoribosyl transferase domain includes the following amino acid consensus sequence [LIVMFYWCTA][LIVM]-[LIVMA]-[LIVMFC]-[DE]-D-[LIVMS][LIVM]-[STAVD]-[STAR]-[GAC]-x[STAR].

The 42967 protein includes the following domains: one predicted N-glycosylation site (PS00001) located at about amino acids 39-42 of SEQ ID NO: 10; one predicted protein kinase C phosphorylation site (PS00005) located at about amino acids 129-131 of SEQ ID NO: 10; one predicted casein kinase II phosphorylation site (PS00006) located at about amino 102-105 of SEQ ID NO: 10; and three predicted N-myristoylation sites (PS00008) located at about amino acids 66-71, 114-119, and 128-133 of SEQ ID NO: 10.

A transferase domain as shown in FIGS. 3, 6, 9, and 15 contains conserved cysteine residues which are likely to form disulfide bonds that affect protein structure.

To identify the presence of a transferase domain in a transferase protein, and make the determination that a protein of interest has a particular profile, the amino acid sequence of the protein is searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for MELPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al.(1990) Meth. Enzymol. 183:146-159; Gribskov et aL (1987) Proc. Natl. Acad. Sci. USA 84:4355-4358; Krogh et al.(1994) J. Mol. Biol. 235:1501-1531; and Stultz et al.(1993) Protein Sci. 2:305-314, the contents of which are incorporated herein by reference. A search was performed against the HMM database resulting in the identification of a transferase domain in the amino acid sequence of human transferase at about residues 105-399 and 4-423 of SEQ ID NO: 2 (see FIG. 3); 4-171 of SEQ ID NO: 4 (see FIG. 6); 82-285 of SEQ ID NO: 6 (see FIG. 9); and 23-171 of SEQ ID NO: 10 (see FIG. 15).

Post-translational modification sites are identified by using Prosite software, Release 12.2 of February 1995, to modify sites as shown in FIGS. 3, 6, 9, 12, and 15.

Isolated proteins of the present invention, preferably transferase proteins, have an amino acid sequence sufficiently homologous to the amino acid sequence of SEQ ID NO: 2, 4, 6, 8, or 10, or are encoded by a nucleotide sequence sufficiently homologous to SEQ ID NO: 1, 3, 5, 7, or 9. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 50% homology, preferably 60% homology, more preferably 70%-80%, and even more preferably 90-95% homology across the amino acid sequences of the domains and contain at least one and preferably two structural domains or motifs, are defined herein as sufficiently homologous. Furthermore, amino acid or nucleotide sequences which share at least 50%, preferably 60%, more preferably 70-80%, or 90-95% homology and share a common functional activity are defined herein as sufficiently homologous.

As used interchangeably herein, a “transferase activity”, “biological activity of transferase” or “functional activity of transferase”, refers to an activity exerted by a transferase protein, polypeptide or nucleic acid molecule on a transferase responsive cell or on a transferase protein substrate, as determined in vivo or in vitro, according to standard techniques. In one embodiment, a transferase activity is a direct activity, such as an association with a transferase target molecule. As used herein, a “target molecule” or “binding partner” is a molecule with which a transferase protein binds or interacts in nature, such that transferase-mediated function is achieved. A transferase target molecule can be a non-transferase molecule or a transferase protein or polypeptide of the present invention. In an exemplary embodiment, a transferase target molecule is a transferase substrate or receptor. A transferase activity can also be an indirect activity, such as a cellular signaling activity mediated by interaction of the transferase protein with a transferase substrate or receptor. Preferably, a transferase activity is the ability to act as a growth regulatory factor and to modulate cell proliferation, differentiation, migration, apoptosis, and/or angiogenesis.

Accordingly, another embodiment of the invention features isolated transferase proteins and polypeptides having a transferase activity. Preferred proteins are transferase proteins including at least one transferase domain as shown in FIGS. 3, 6, 9, 12, and 15, and, preferably, having a transferase activity. Further preferred proteins include at least one transferase domain as shown in FIGS. 3, 6, 9, 12, and 15, and are, preferably, encoded by a nucleic acid molecule having a nucleotide sequence which hybridizes under stringent hybridization conditions to a nucleic acid molecule comprising the nucleotide sequence of SEQ ID NO: 1,3,5,7, or 9.

The nucleotide sequence of the isolated human transferase cDNA and the predicted amino acid sequence of the human transferase polypeptide correspond to the sequences shown in FIGS. 1, 4, 7, 10 and 13 and in SEQ ID NOs: 1, 3, 5, 7, and 9, and SEQ ID NOs: 2, 4, 6, 8, and 10 respectively. Human transferase genes as shown in FIGS. 1, 4, 7, 10 and 13, which are approximately 1892, 1001, 1832, 5426, and 602 nucleotides in length respectively, encodes a protein having a molecular weight of approximately 47, 20, 41, 47, 19 kD respectively and which are approximately 425, 184, 376, 423, and 173 amino acid residues in length respectively.

Various aspects of the invention are described in further detail in the following subsections:

I. Isolated Nucleic Acid Molecules

One aspect of the invention pertains to isolated nucleic acid molecules that encode transferase proteins or biologically active portions thereof, as well as nucleic acid fragments sufficient for use as hybridization probes to identify transferase -encoding nucleic acid molecules (e.g., transferase MRNA) and fragments for use as PCR primers for the amplification or mutation of transferase nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is doublestranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated transferase nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO: 1, 3, 5, 7, or 9, or a portion thereof, can be isolated using standard molecular biology techniques and the sequence information provided herein. Using all or a portion of the nucleic acid sequence of SEQ ID NO: 1, 3, 5, 7, or 9 as hybridization probes, transferase nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y. 1989).

Moreover, a nucleic acid molecule encompassing all or a portion of SEQ ID NO: 1, 3, 5, 7, or 9 can be isolated by the polyrnerase chain reaction (PCR) using synthetic oligonucleotide primers designed based upon the sequence of SEQ ID NO: 1, 3, 5, 7, or 9.

A nucleic acid of the invention can be amplified using cDNA, MRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to transferase nucleotide sequences can be prepared by standard synthetic techniques, e.g., using an automated DNA synthesizer.

In one embodiment, an isolated nucleic acid molecules of the invention comprise cDNA. The sequences of SEQ ID NO: 1, 3, 5, 7, or 9 correspond to the human transferase cDNA.

The cDNA corresponding to SEQ ID NO: 1 in FIG. 1 comprises sequences encoding the human transferase protein (i.e., “the coding region”, from nucleotides 1-1278 starting at ATG), as well as 5′ untranslated sequences (279 nucleotides before the coding region) and 3′ untranslated sequences (335 nucleotides after the coding region).

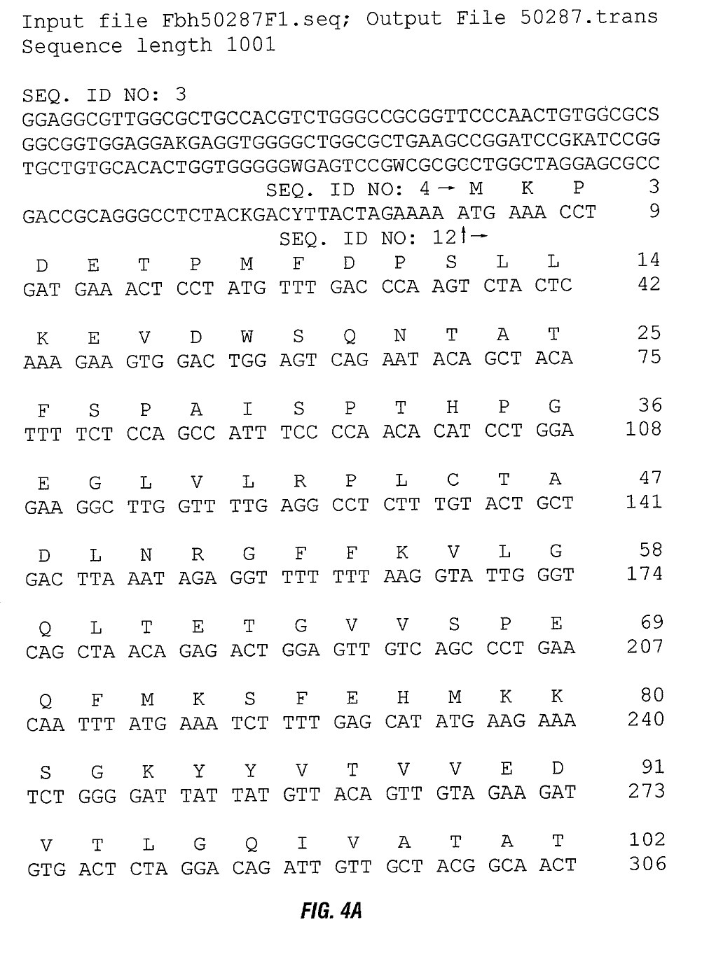

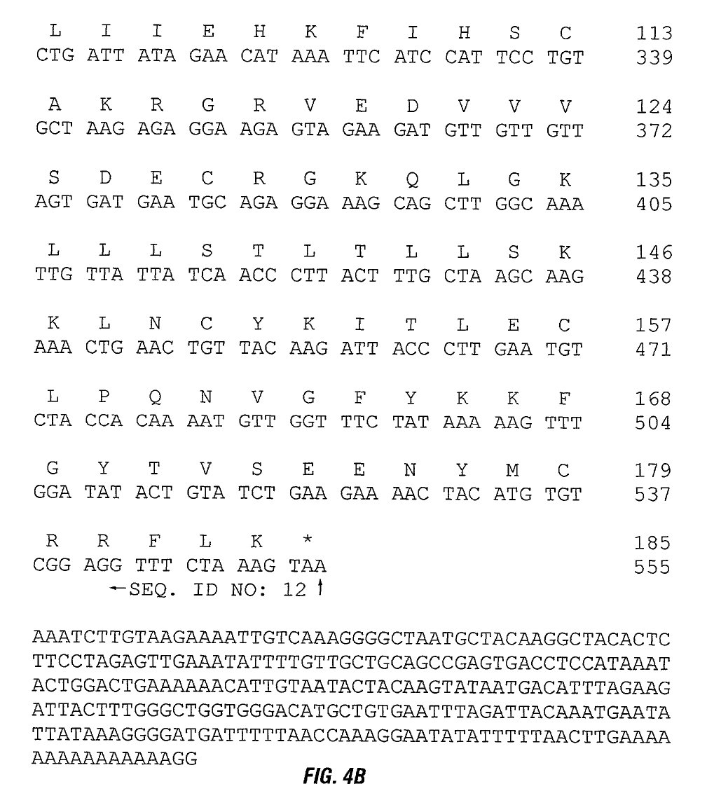

The cDNA corresponding to SEQ ID NO: 3 in FIG. 4 comprises sequences encoding the human transferase protein (i.e., “the coding region”, from nucleotides 1-555 starting at ATG), as well as 5′ untranslated sequences (183 nucleotides before the coding region) and 3′ untranslated sequences (263 nucleotides after the coding region).

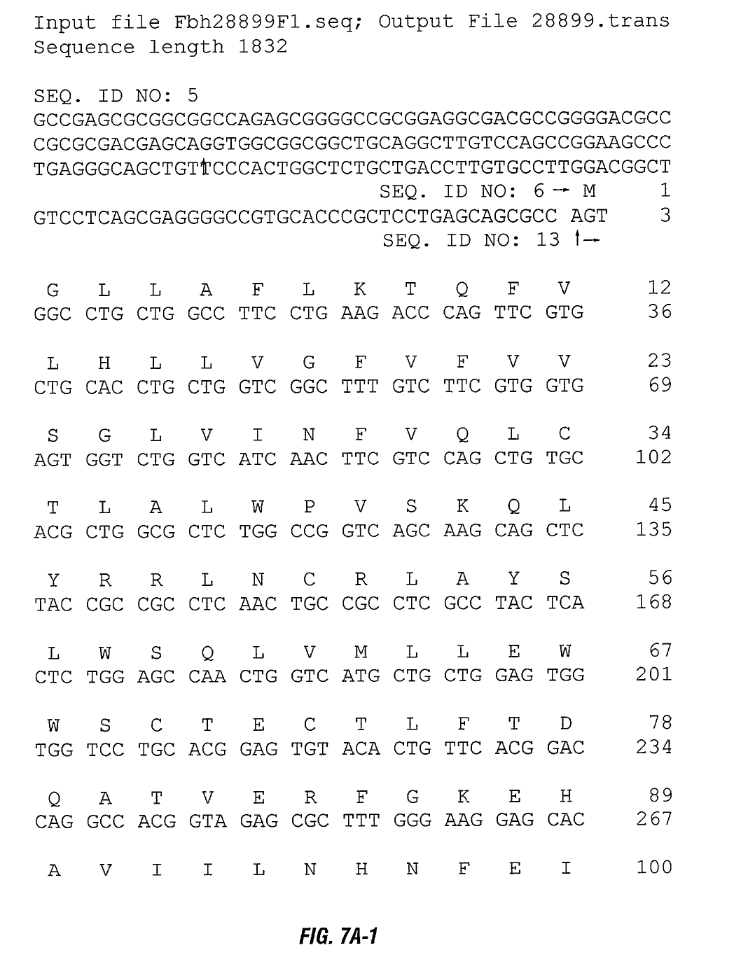

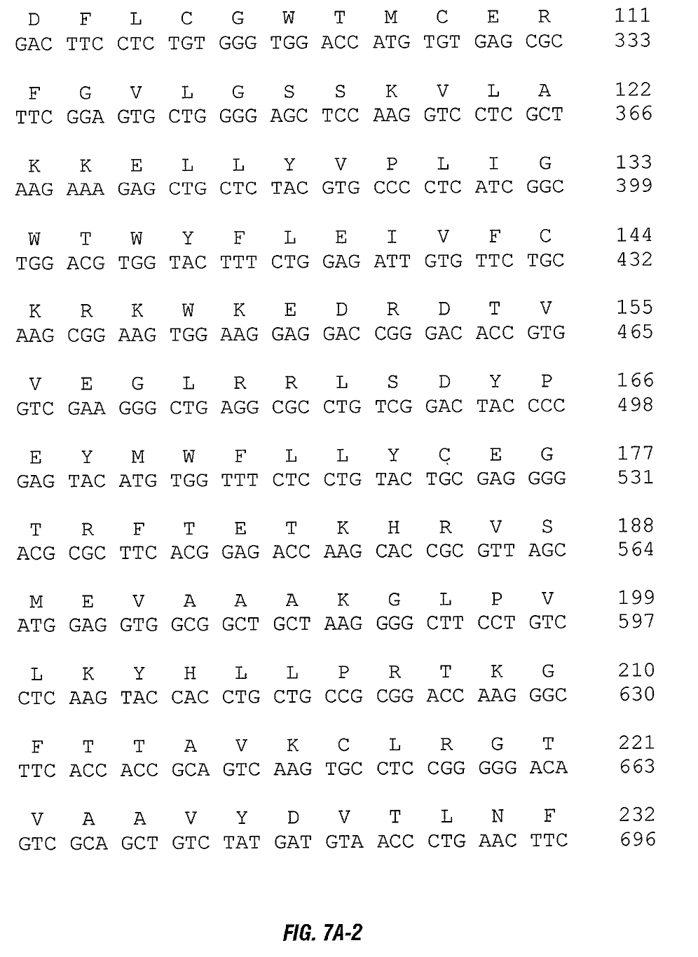

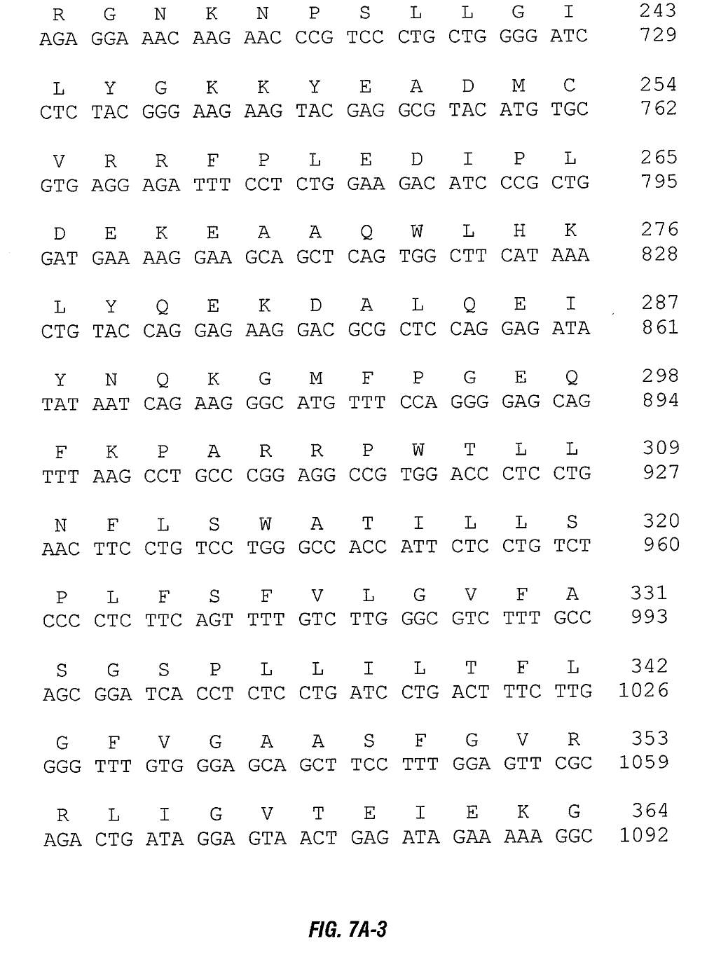

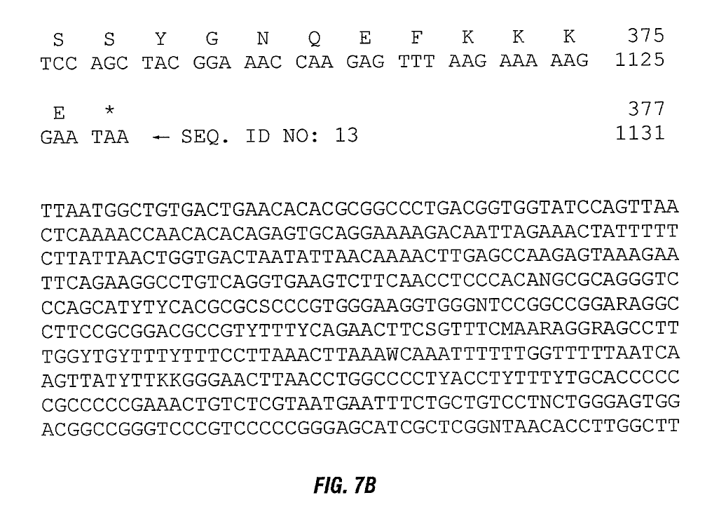

The cDNA corresponding to SEQ ID NO: 5 in FIG. 7 comprises sequences encoding the human transferase protein (i.e., “the coding region”, from nucleotides 1-1131 starting at ATG), as well as 5′ untranslated sequences (191 nucleotides before the coding region) and 3′ untranslated sequences (510 nucleotides after the coding region).

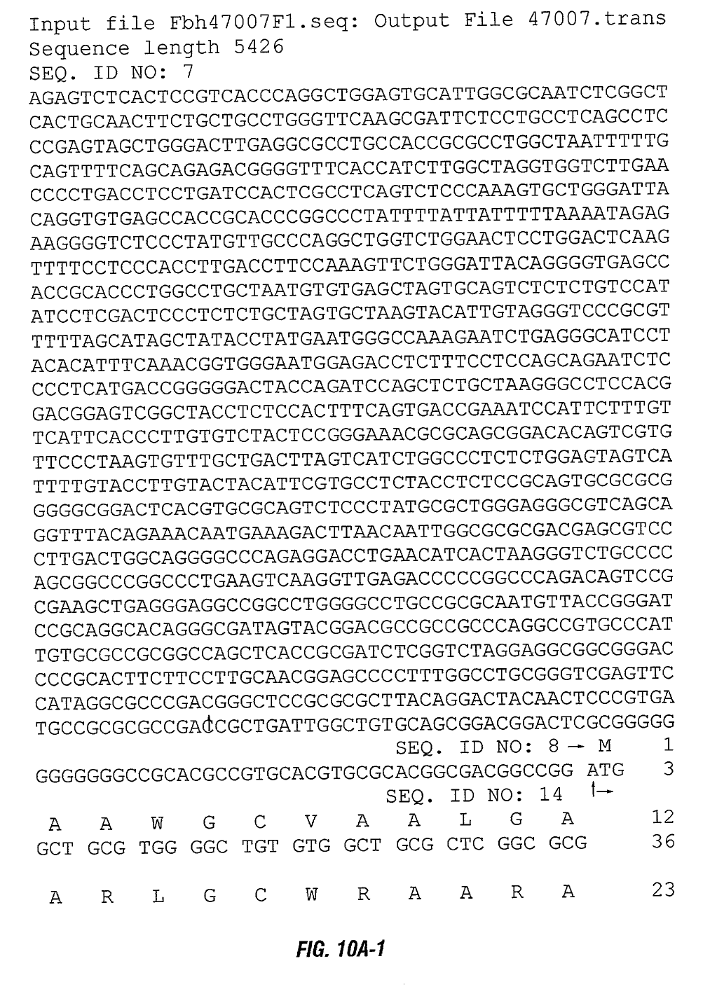

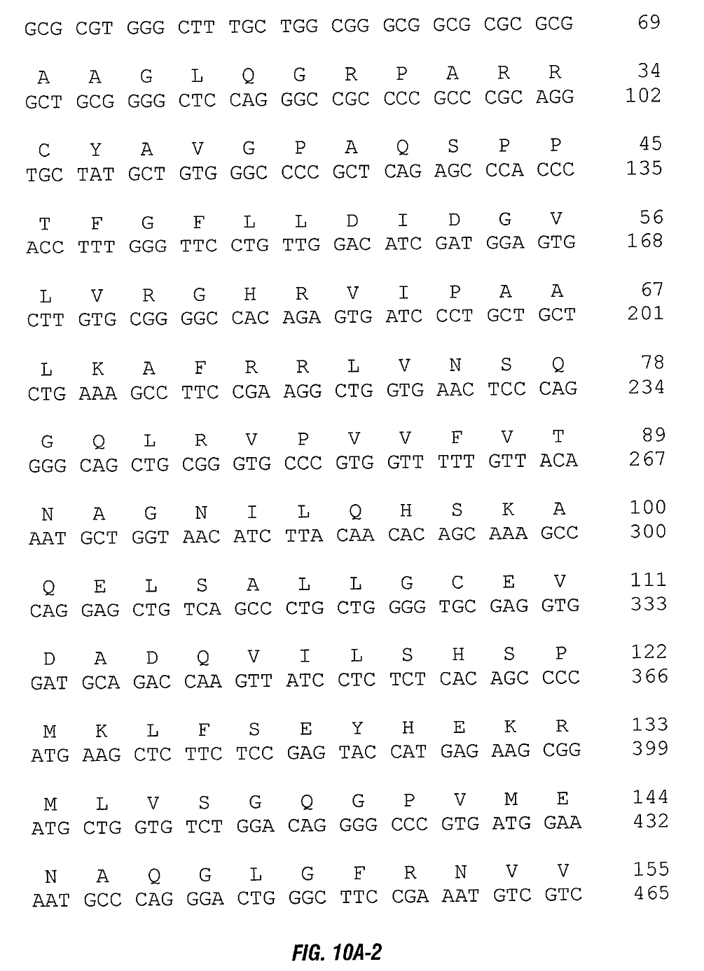

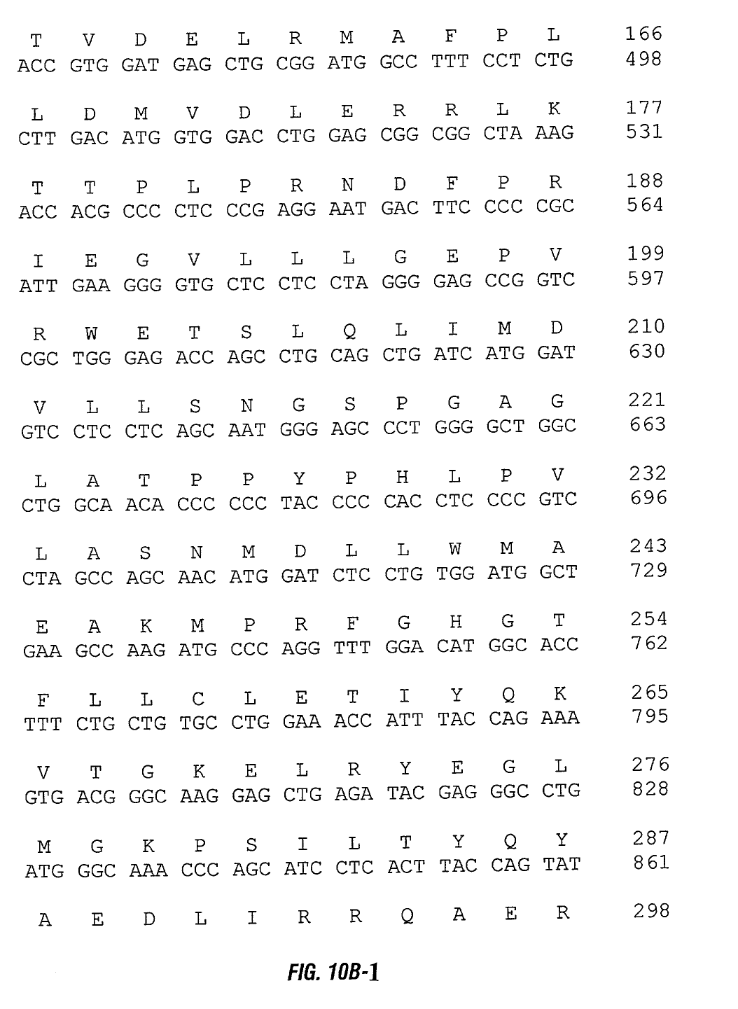

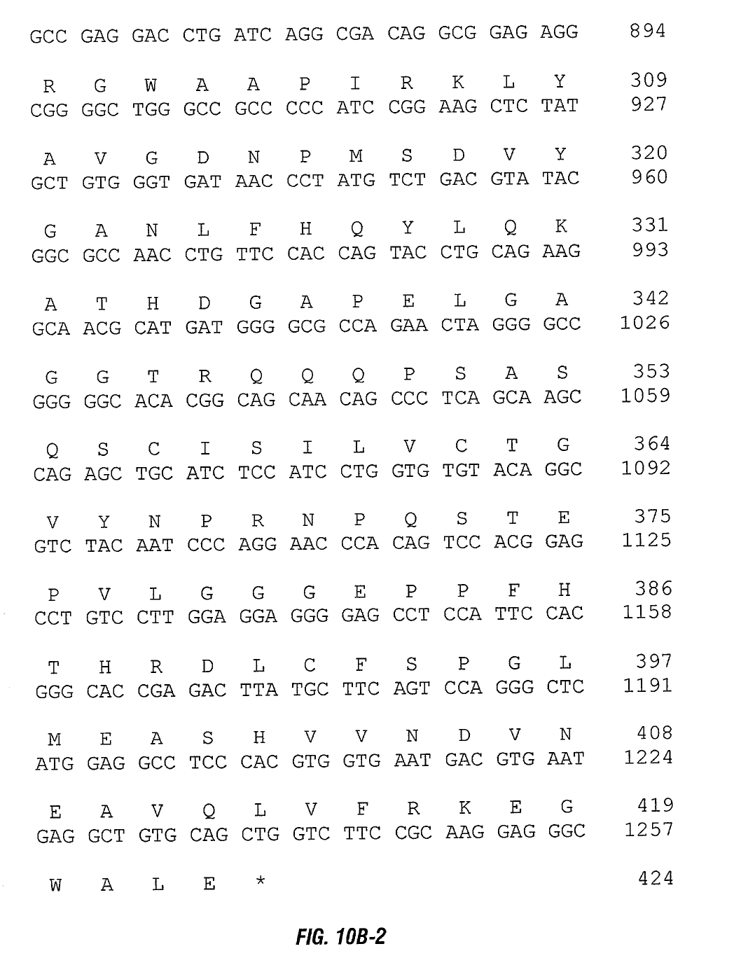

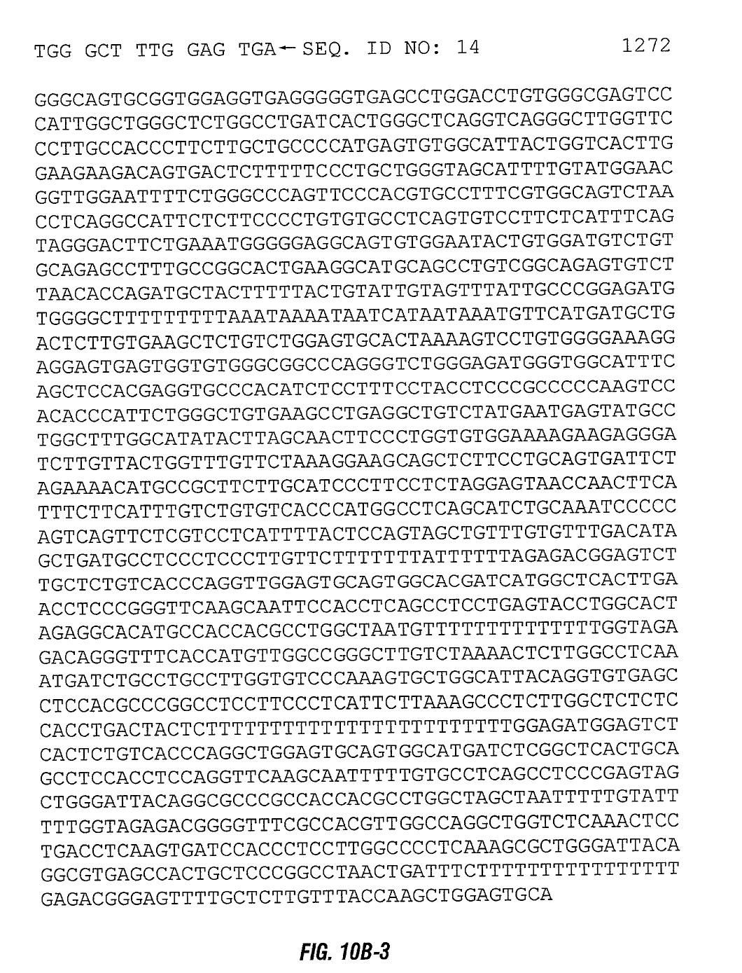

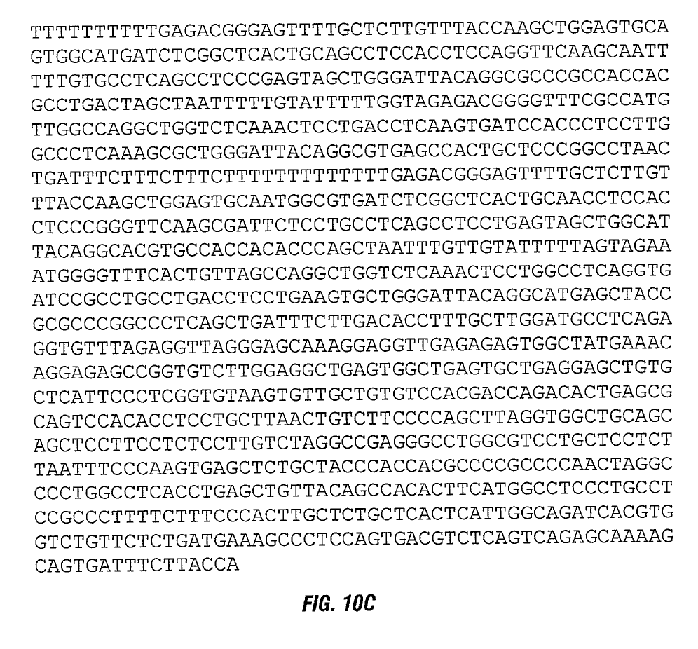

The cDNA corresponding to SEQ ID NO: 7 in FIG. 10 comprises sequences encoding the human transferase protein (i.e., “the coding region”, from nucleotides 1-1272 starting at ATG), as well as 5′ untranslated sequences (1392 nucleotides before the coding region) and 3′ untranslated sequences (2762 nucleotides after the coding region).

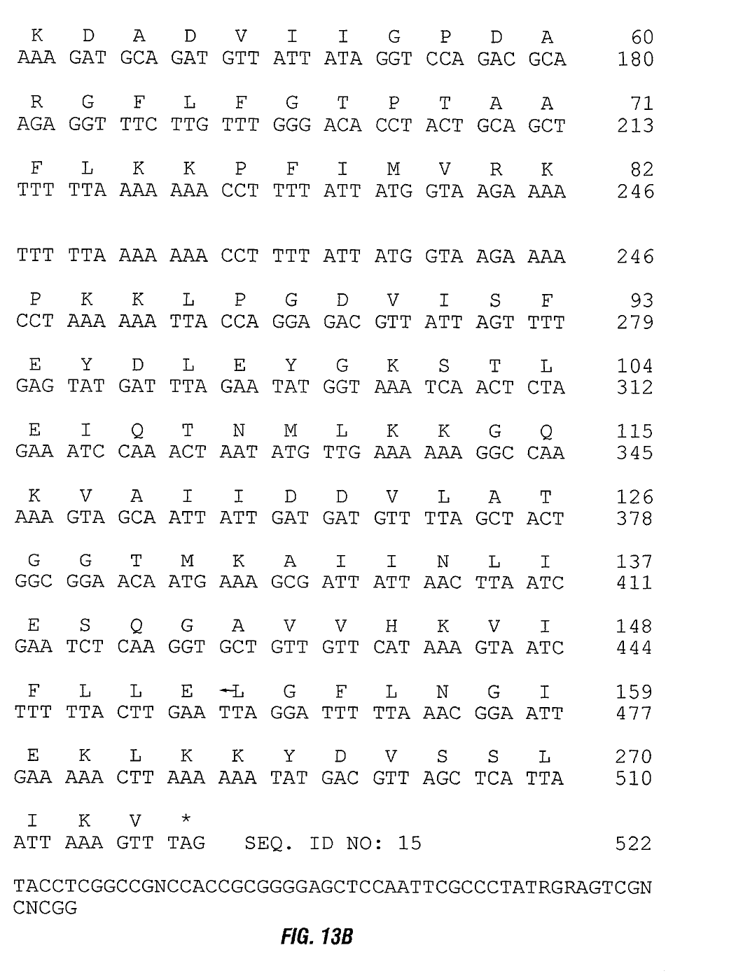

The cDNA corresponding to SEQ ID NO: 10 in FIG. 13 comprises sequences encoding the human transferase protein (i.e., “the coding region”, from nucleotides 1-522 starting at ATG), as well as 5′ untranslated sequences (25 nucleotides before the coding region) and 3′ untranslated sequences (55 nucleotides after the coding region).

In one embodiment, an isolated nucleic acid molecule of the invention comprises a nucleic acid molecule which is a complement of the nucleotide sequence shown in SEQ ID NO: 1, 3, 5, 7, or 9, or a portion of any of these nucleotide sequences. A nucleic acid molecule which is complementary to the nucleotide sequence shown in SEQ ID NO: 1, 3, 5, 7, or 9 is one which is sufficiently complementary to the nucleotide sequence shown in SEQ ID NO: 1, 3, 5, 7, or 9, such that it can hybridize to the nucleotide sequence shown in SEQ ID NO: 1, 3, 5, 7, or 9, thereby forming a stable duplex.

In one embodiment, an isolated nucleic acid molecule of the present invention comprises a nucleotide sequence which is at least about 50%, 55%, 60%, 65%, 70%, 75%, 80%, 85%, 90%, 95%, 98% or more homologous to the entire length of the nucleotide sequence shown in SEQ ID NO: 1, 3, 5, 7, or 9, or a portion of any of these nucleotide sequences.

Moreover, the nucleic acid molecule of the invention can comprise only a portion of the nucleic acid sequence of SEQ ID NO: 1, 3, 5, 7, or 9, for example, a fragment which can be used as a probe or primer or a fragment encoding a portion of a transferase protein, e.g., an immunogenic or biologically active portion of a transferase protein. The nucleotide sequence determined from the cloning of the transferase gene allows for the generation of probes and primers designed for use in identifying and/or cloning other transferase family members, as well as transferase homologues from other species. The probe/primer typically comprises substantially purified oligonucleotide. The oligonucleotide typically comprises a region of nucleotide sequence that hybridizes under stringent conditions to at least about 7, 12 or 15, preferably about 20 or 25, more preferably about 30, 35, 40, 45, 50, 55, 60, 65, or 75 consecutive nucleotides of a sense or antisense sequence of SEQ ID NO: 1, 3, 5, 7, or 9, or of a naturally occurring allelic variant or mutant of SEQ ID NO: 1, 3, 5, 7, or 9. In an exemplary embodiment, a nucleic acid molecule of the present invention comprises a nucleotide sequence which is greater than 50, 60, 70, 80, 90, 100, 150, 200, 300, 400, 500, 549, 549-600, (for SEQ ID NO: 1, 3, 5, 7, 9) 600-650, 650-700, 700-750, 750-800, 800-850, 850-900, 900-950, 950-1000 (for SEQ ID NO: 1, 3, 5, 7) 1000-1100, 1100-1200, 1200-1300, 1300-1400, 1400-1500, 1500-1600, 1600-1700, 1700-1800, (for SEQ ID NO: 1, 5, 7) 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, 4400-4500, 4500-4600, 4600-4700, 4700-4800, 4800-4900, 4900-5000, 5000-5100, 5100-5200, 5200-5300, 5300-5400, (for SEQ ID NO: 7) or more nucleotides in length and hybridizes under stringent hybridization conditions to a nucleic acid molecule of SEQ ID NO: 1, 3, 5, 7, or 9.

The invention further encompasses nucleic acid molecules that differ from the nucleotide sequence shown in SEQ ID NO: 1, 3, 5, 7, or 9 due to degeneracy of the genetic code and thus encode the same transferase proteins as those encoded by the nucleotide sequence shown in SEQ ID NO: 1, 3, 5, 7, or 9. In another embodiment, an isolated nucleic acid molecule of the invention has a nucleotide sequence encoding a protein having an amino acid sequence shown in SEQ ID NO: 2, 4, 6, 8, or 10.

In addition to the transferase nucleotide sequences shown in SEQ ID NO: 1, 3, 5, 7, and 9, it will be appreciated by those skilled in the art that DNA sequence polymorphisms that lead to changes in the amino acid sequences of the transferase proteins may exist within a population (e.g., the human population). Such genetic polymorphism in the transferase genes may exist among individuals within a population due to natural allelic variation. As used herein, the terms “gene” and “recombinant gene” refer to nucleic acid molecules which include an open reading frame encoding a transferase protein, preferably a mammalian transferase protein, and can further include non-coding regulatory sequences, and introns.

Allelic variants of transferase, e.g., human transferase, include both functional and nonfunctional transferase proteins. Functional allelic variants are naturally occurring amino acid sequence variants of the transferase protein within a population that maintain the ability to bind a transferase receptor or substrate, and/or modulate cell growth and migration mechanisms. Functional allelic variants will typically contain only conservative substitution of one or more amino acids of SEQ ID NO: 2, 4, 6, 8, or 10, or substitution, deletion or insertion of non-critical residues in non-critical regions of the protein. Non-functional allelic variants are naturally occurring amino acid sequence variants of the transferase, e.g., human transferase, protein within a population that do not have the ability to either bind a transferase receptor or substrate, or modulate cell growth or migration mechanisms. Non-functional allelic variants will typically contain a non-conservative substitution, a deletion, or insertion, or premature truncation of the amino acid sequence of SEQ ID NO: 2, 4, 6, 8, or 10, or a substitution, insertion, or deletion in critical residues or critical regions of the protein.

The present invention further provides orthologues of the human transferase protein. Orthologues of the human transferase protein are proteins that are isolated from non-human organisms and possess the same transferase receptor or substrate binding mechanisms, and/or modulation of cell growth or migration mechanisms of the human transferase protein. Orthologues of the human transferase protein can readily be identified as comprising an amino acid sequence that is substantially homologous to SEQ ID NO: 2, 4, 6, 8, or 10.

Moreover, nucleic acid molecules encoding other transferase family members and, thus, which have a nucleotide sequence which differs from the transferase sequences of SEQ ID NO: 1, 3, 5, 7, or 9 are intended to be within the scope of the invention. For example, another transferase cDNA can be identified based on the nucleotide sequence of human transferase. Moreover, nucleic acid molecules encoding transferase proteins from different species, and which, thus, have a nucleotide sequence which differs from the transferase sequences of SEQ ID NO: 1, 3, 5, 7, or 9 are intended to be within the scope of the invention. For example, a mouse transferase cDNA can be identified based on the nucleotide sequence of a human transferase.

Nucleic acid molecules corresponding to natural allelic variants and homologues of the transferase cDNAs of the invention can be isolated based on their homology to the transferase nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the transferase cDNAs of the invention can further be isolated by mapping to the same chromosome or locus as the transferase gene. Accordingly, in another embodiment, an isolated nucleic acid molecule of the invention is at least 7, 15, 20, 25, 30 or more nucleotides in length and hybridizes under stringent conditions to the nucleic acid molecule comprising the nucleotide sequence of SEQ ID NO: 1, 3, 5, 7, or 9. In other embodiment, the nucleic acid is at least 30, 50, 100, 150, 200, 250, 253, 300, 350, 400, 450, 500, 550, 600, 650, 700, 750, or 800 nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6X sodium chloride/sodium citrate (SSC) at about 45° C., followed by one or more washes in 0.2× SSC, 0.1% SDS at 50° C. Another example of stringent hybridization conditions are hybridization in 6X sodium chloride/sodium citrate (SSC) at about 45° C., followed by one or more washes in 0.2× SSC, 0.1% SDS at 55° C. A further example of stringent hybridization conditions are hybridization in 6× sodium chloride/sodium citrate (SSC) at about 45° C., followed by one or more washes in 0.2× SSC, 0.1% SDS at 60° C. Preferably, stringent hybridization conditions are hybridization in 6× sodium chloride/sodium citrate (SSC) at about 45° C., followed by one or more washes in 0.2× SSC, 0.1% SDS at 65° C., more preferably stringent hybridization conditions are hybridization in 0.5 M sodium phosphate, 7% SDS at 65° C., followed by one or more washes in 0.2× SSC, 0.1% SDS at 65° C. Preferably, an isolated nucleic acid molecule of the invention that hybridizes under stringent conditions to the sequence of SEQ ID NO: 1, 3, 5, 7, or 9 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the transferase sequences that may exist in the population, the skilled artisan will further appreciate that changes can be introduced by mutation into the nucleotide sequences of SEQ ID NO: 1, 3, 5, 7, or 9, thereby leading to changes in the amino acid sequence of the encoded transferase proteins, without altering the functional ability of the transferase proteins. For example, nucleotide substitutions leading to amino acid substitutions at “non-essential” amino acid residues can be made in the sequence of SEQ ID NO: 1, 3, 5, 7, or 9. A “non-essential” amino acid residue is a residue that can be altered from the wild-type sequence of transferase (e.g., the sequence of SEQ ID NO: 2, 4, 6, 8, or 10) without altering the biological activity, whereas an “essential” amino acid residue is required for biological activity. For example, amino acid residues that are conserved among the transferase proteins of the present invention, e.g., those present in the transferase superfamily variant motif, the transferase disulfide knot-like domain, or the CUB domain, are predicted to be particularly unamenable to alteration. Furthermore, additional amino acid residues that are conserved between the transferase proteins of the present invention and other members of the transferase family are not likely to be amenable to alteration.

Accordingly, another aspect of the invention pertains to nucleic acid molecules encoding transferase proteins that contain changes in amino acid residues that are not essential for activity. Such transferase proteins differ in amino acid sequence from SEQ ID NO: 2, 4, 6, 8, or 10, yet retain biological activity. In one embodiment, the isolated nucleic acid molecule comprises a nucleotide sequence encoding a protein, wherein the protein comprises an amino acid sequence at least about 50%, 55%, 60%, 65%, 70%, 75%, 80%, 85%, 90%, 95%, 98% or more homologous to SEQ ID NO: 2, 4, 6, 8, or 10.

An isolated nucleic acid molecule encoding a transferase protein homologous to the protein of SEQ ID NO: 2, 4, 6, 8, or 10 can be created by introducing one or more nucleotide substitutions, additions or deletions into the nucleotide sequence of SEQ ID NO: 1, 3, 5, 7, or 9 such that one or more amino acid substitutions, additions or deletions are introduced into the encoded protein. Mutations can be introduced into SEQ ID NO: 1, 3, 5, 7, or 9 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a transferase protein is preferably replaced with another amino acid residue from the same side chain family. Alternatively, in another embodiment, mutations can be introduced randomly along all or part of a transferase coding sequence, such as by saturation mutagenesis, and the resultant mutants can be screened for transferase biological activity to identify mutants that retain activity. Following mutagenesis of SEQ ID NO: 1, 3, 5, 7, or 9, the encoded protein can be expressed recombinantly and the activity of the protein can be determined.

In a preferred embodiment, a mutant transferase protein can be assayed for the ability to (1) interact with a non-transferase protein molecule, e.g., a transferase substrate or receptor; (2) activate a transferase-dependent signal transduction pathway; (3) modulate cell proliferation, differentiation, migration and/or apoptosis mechanisms; or (4) modulate angiogenic processes.

In addition to the nucleic acid molecules encoding transferase proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire transferase coding strand, or to only a portion thereof. In one embodiment, an antisense nucleic acid molecule is antisense to a “coding region” of the coding strand of a nucleotide sequence encoding transferase. The term “coding region” refers to the region of the nucleotide sequence comprising codons which are translated into amino acid residues (e.g., the coding region of human transferase is shown as that portion of SEQ ID NO: 1, 3, 5, 7, or 9 that corresponds to the amino acid residues of SEQ ID NO: 2, 4, 6, 8, or 10, respectively, which consist of SEQ ID NO: 11, 12, 13, 14 or 15, not including the terminal codon). In another embodiment, the antisense nucleic acid molecule is antisense to a “noncoding region” of the coding strand of a nucleotide sequence encoding transferase. The term “noncoding region” refers to 5′ and 3′ sequences which flank the coding region that are not translated into amino acids (i.e., also referred to as 5′ and 3′ untranslated regions).

Given the coding strand sequences encoding transferase disclosed herein (e.g., SEQ ID NO: 1, 3, 5, 7, or 9), antisense nucleic acids of the invention can be designed according to the rules of Watson and Crick base pairing. The antisense nucleic acid molecule can be complementary to the entire coding region of transferase MRNA, but more preferably is an oligonucleotide which is antisense to only a portion of the coding or noncoding region of transferase mRNA. For example, the antisense oligonucleotide can be complementary to the region surrounding the translation start site of transferase MRNA. An antisense oligonucleotide can be, for example, about 7, 10, 15, 20, 25, 30, 35, 40, 45, 50, 55, 60, 65, 70, 75, 80, or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a transferase protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an MRNA, to which they have a complementary region. Thus, ribozymes (e.g., hammerhead ribozymes (described in Haselhoff and Gerlach (1988) Nature 334:585-591)) can be used to catalytically cleave transferase MRNA transcripts to thereby inhibit translation of transferase mRNA. A ribozyme having specificity for a transferase-encoding nucleic acid can be designed based upon the nucleotide sequence of a transferase cDNA disclosed herein (i.e., corresponding to SEQ ID NO: 1, 3, 5, 7, or 9). For example, a derivative of a Tetrahymena L-19 IVS RNA can be constructed in which the nucleotide sequence of the active site is complementary to the nucleotide sequence to be cleaved in a transferase-encoding mRNA. See, e.g., Cech et al. U.S. Pat. No. 4,987,071; and Cech et al. U.S. Pat. No. 5,116,742. Alternatively, transferase MnRNA can be used to select a catalytic RNA having a specific ribonuclease activity from a pool of RNA molecules. See, e.g., Bartel, D. and Szostak, J. W. (1993) Science 261:1411-1418.

Alternatively, transferase gene expression can be inhibited by targeting nucleotide sequences complementary to the regulatory region of the transferase (e.g., the transferase promoter and/or enhancers) to form triple helical structures that prevent transcription of the transferase gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the transferase nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93:14670-675.

PNAs of transferase nucleic acid molecules can be used in therapeutic and diagnostic applications. For example, PNAs can be used as antisense or antigene agents for sequencespecific modulation of gene expression by, for example, inducing transcription or translation arrest or inhibiting replication. PNAs of transferase nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of transferase can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of transferase nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17):3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17:5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. NatL. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

The invention also provides detectably labeled oligonucleotide primer and probe molecules. Typically, such labels are chemiluminescent, fluorescent, radioactive, or calorimetric to permit ease of detection. Such labels and the criteria by which one label would be selected over another are well known to those skilled in the art.

One variety of detectable label which is particularly well-suited to the methods of the invention is a molecular beacon, since this technology permits detection of the label only in the instance where the oligonucleotide molecule bearing the molecular beacon is hybridized to a target sequence. The invention therefore includes molecular beacon oligonucleotide primer and probe molecules having at least one region which is complementary to a transferase nucleic acid of the invention, such that the molecular beacon is useful for quantitating the presence of the transferase nucleic acid of the invention in a sample. A “molecular beacon” oligonucleotide is a nucleic acid comprising a pair of complementary regions and having a fluorophore and fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid are not annealed with one another, such as is the case when the primer or probe is hybridized to its target sequence, the fluorophore and quencher are distanced, and the fluorescence of the fluorophore is quenched to a lesser degree. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

II. Isolated Transferase Proteins and Anti-transferase Antibodies

One aspect of the invention pertains to isolated transferase proteins, and biologically active portions thereof, as well as polypeptide fragments suitable for use as immunogens to raise anti-transferase antibodies. In one embodiment, native transferase proteins can be isolated from cells or tissue sources by an appropriate purification scheme using standard protein purification techniques. In another embodiment, transferase proteins are produced by recombinant DNA techniques. Alternative to recombinant expression, a transferase protein or polypeptide can be synthesized chemically using standard peptide synthesis techniques.

An “isolated” or “purified” protein or biologically active portion thereof is substantially free of cellular material or other contaminating proteins from the cell or tissue source from which the transferase protein is derived, or substantially free from chemical precursors or other chemicals when chemically synthesized. The language “substantially free of cellular material” includes preparations of transferase protein in which the protein is separated from cellular components of the cells from which it is isolated or recombinantly produced. In one embodiment, the language “substantially free of cellular material” includes preparations of transferase protein having less than about 30% (by dry weight) of non-transferase protein (also referred to herein as a “contaminating protein”), more preferably less than about 20% of nontransferase protein, still more preferably less than about 10% of non-transferase protein, and most preferably less than about 5% non-transferase protein. When the transferase protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of transferase protein in which the protein is separated from chemical precursors or other chemicals which are involved in the synthesis of the protein. In one embodiment, the language “substantially free of chemical precursors or other chemicals” includes preparations of transferase protein having less than about 30% (by dry weight) of chemical precursors or non-transferase chemicals, more preferably less than about 20% chemical precursors or non-transferase chemicals, still more preferably less than about 10% chemical precursors or non-transferase chemicals, and most preferably less than about 5% chemical precursors or non-transferase chemicals.

As used herein, a “biologically active portion” of a transferase protein includes a fragment of a transferase protein which participates in an interaction between a transferase molecule and a non-transferase molecule. Biologically active portions of a transferase protein include peptides comprising amino acid sequences sufficiently homologous to or derived from the amino acid sequence of the transferase protein, e.g., the amino acid sequence shown in SEQ ID NO: 2, 4, 6, 8, or 10, which include less amino acids than the full length transferase proteins, and exhibit at least one activity of a transferase protein. Typically, biologically active portions comprise a domain or motif with at least one activity of the transferase protein, e.g., modulating cell growth and/or migration mechanisms. A biologically active portion of a transferase protein can be a polypeptide which is, for example, 10, 25, 50, 100, 200 or more amino acids in length. Biologically active portions of a transferase protein can be used as targets for developing agents which modulate a transferase mediated activity, e.g., a cell proliferation, differentiation, migration, apoptosis, or angiogenic signaling mechanism.

In one embodiment, a biologically active portion of a transferase protein comprises at least one transferase domain as shown in FIGS. 2, 3, 5, 6, 8, 9, 11, 12, 14 and 15. It is to be understood that a preferred biologically active portion of a transferase protein of the present invention may contain at least one amino-, acetyl-, acyl-, phosphatidyl-, or phosphoribosyltransferase domain as shown in FIGS. 2, 3, 5, 6, 8, 9, 11, 12, 14 and 15. Moreover, other biologically active portions, in which other regions of the protein are deleted, can be prepared by recombinant techniques and evaluated for one or more of the functional activities of a native transferase protein.

In a preferred embodiment, the transferase protein has an amino acid sequence shown in SEQ ID NO: 2, 4, 6, 8, or 10. In other embodiments, the transferase protein is substantially homologous to SEQ ID NO: 2, 4, 6, 8, or 10, and retains the functional activity of the protein of SEQ ID NO: 2, 4, 6, 8, or 10, yet differs in amino acid sequence due to natural allelic variation or mutagenesis, as described in detail in subsection I above. Accordingly, in another embodiment, the transferase protein is a protein which comprises an amino acid sequence at least about 50%, 55%, 60%, 65%, 70%, 75%, 80%, 85%, 90%, 95%, 98% or more homologous to SEQ ID NO: 2,4,6, 8,or 10.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the transferase amino acid sequence of SEQ ID NO: 2 having approximately 425 amino acid residues, at least 128, preferably at least 170, more preferably at least 213, even more preferably at least 255, and even more preferably at least 298, 340 or 383 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In a more preferred embodiment, the percent identity between two amino acid or nucleotide sequences is determined using a Blossum 62 matrix, a gap open penalty of 12, a gap extend penalty of 4 and a frameshift gap penalty of 5.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to transferase nucleic acid molecules of the invention. BLAST protein searches can be performed with the XBLAST program, score=50, word length=3 to obtain amino acid sequences homologous to transferase protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides transferase chimeric or fusion proteins. As used herein, a transferase “chimeric protein” or “fusion protein” comprises a transferase polypeptide operatively linked to a non-transferase polypeptide. An “transferase polypeptide” refers to a polypeptide having an amino acid sequence corresponding to transferase, whereas a “nontransferase polypeptide” refers to a polypeptide having an amino acid sequence corresponding to a protein which is not substantially homologous to the transferase protein, e.g., a protein which is different from the transferase protein and which is derived from the same or a different organism. Within a transferase fusion protein the transferase polypeptide can correspond to all or a portion of a transferase protein. In a preferred embodiment, a transferase fusion protein comprises at least one biologically active portion of a transferase protein. In another preferred embodiment, a transferase fusion protein comprises at least two biologically active portions of a transferase protein. Within the fusion protein, the term “operatively linked” is intended to indicate that the transferase polypeptide and the non-transferase polypeptide are fused in-frarne to each other. The non-transferase polypeptide can be fused to the N-terminus or C-terminus of the transferase polypeptide.

For example, in one embodiment, the fusion protein is a GST-transferase fusion protein in which the transferase sequences are fused to the C-terminus of the GST sequences. Such fusion proteins can facilitate the purification of recombinant transferase. In another embodiment, the fusion protein is a transferase protein containing a heterologous signal sequence at its N-terminus. In certain host cells (e.g., mammalian host cells), expression and/or secretion of transferase can be increased through use of a heterologous signal sequence.

The transferase fusion proteins of the invention can be incorporated into pharmaceutical compositions and administered to a subject in vivo. The transferase fusion proteins can be used to affect the bioavailability of a transferase substrate. Use of transferase fusion proteins may be useful therapeutically for the treatment of disorders caused by, for example, (i) aberrant modification or mutation of a gene encoding a transferase protein; (ii) mis-regulation of the transferase gene; and (iii) aberrant post-translational modification of a transferase protein.

Moreover, the transferase-fusion proteins of the invention can be used as immunogens to produce anti-transferase antibodies in a subject, to purify transferase ligands and in screening assays to identify molecules which inhibit the interaction of transferase with a transferase substrate.

Preferably, a transferase chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A transferase-encoding nucleic acid can be cloned an expression vector such that the fusion moiety is linked in-frame to the transferase proteins

The present invention also pertains to variants of the transferase proteins which function as either transferase agonist (mimetics) or as transferase antagonist. Variants of the transferase proteins can be generated by mutagenesis, e,g., discrete point mutation or truncation of a transferase protein. An agonist of the transferase proteins can retain substantially the same, or a subset, of the biological activities of the naturally occurring from of a transferase protein. An antagonist of a transferase protein can inhibit one or more of the activities of the naturally occurring form of the transferase protein by, for example, competitively modulating a transferase-mediate activity of a transferase protein. Thus, specific biological effects can be elicited by treatment with a variant of limited function. In one embodiment of a subject with a variant having a subject of the biological activities of the naturally occurring form of the protein has fewer side effects in a subject relative to treatment with the naturally occurring form of the transferase-protein.

In one embodiment, variants of transferase protein which functions as either transferase agonist (mimetics) or as transferase antagonist can be identified by screening combinatorial libraries of mutantns, e,g., truncation mutants, of a transferase protein for transferase protein agonist or antagonist activity. In one embodiment, a variegated library of transferase variants is generated by combinatorial mutagenesis at the nucleic acid level and is encoded by, for variegated gene library. A variegated library of transferase variants can be produced by, for example, enzymatically ligating a mixture of synthetic oigonucleotides into gene sequences such that a degenerate set of potential transferase sequences is expresible as individual polypectides, or alternatively, as a set of larger fusion of proteins (e,g., for phage display) containig the set of transferase sequences therein. There are a variety of methods which can be used to produce libraries of potential transferase variants from a degenerate oligonucleotide sequence. Chemical synthesis of a degenerate gene sequence can be perform in an automatic DNA synthetizer, and the synthetic gene then ligated into an appropiate expression vector. Use of a degenerate set of genes allows for the provision, in one mixture, of all of the sequence encoding the desired set of potential transferase sequences, Methods for synthetizing degenerate oligonucleotides are known in the art (see, e,g., Narang, S.A (1983) Tetrahedron 39:3; Itakura et al. (1984) Annu. Rev. Biochem. 53:323; Itakura et al. (1984) Science 198:1056; Ike et al. (1983)NucleicAcidRes. 11:477.

In addition, libraries of fragments of a transferase protein coding sequence can be used to generate a variegated population of transferase fragments for screening and subsequent selection of variants of a transferase protein. In one embodiment, a library of coding sequence fragments can be generated by treating a double stranded PCR fragment of a transferase coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the transferase protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of transferase proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify transferase variants (Arkin and Yourvan (1992) Proc. Natl. Acad. Sci. USA 89:7811-7815; Delgrave et al. (1993) Protein Engineering 6(3):327-331).

In one embodiment, cell based assays can be exploited to analyze a variegated transferase library. For example, a library of expression vectors can be transfected into a cell line, e.g., an endothelial cell line, which ordinarily responds to transferase in a particular transferase substrate-dependent manner. The transfected cells are then contacted with transferase and the effect of the expression of the mutant on signaling by the transferase substrate can be detected, e.g., by measuring intracellular calcium and inositol 1,4,5-trisphosphate (IP3) levels, cell growth, and cell migration. Plasmid DNA can then be recovered from the cells which score for inhibition, or alternatively, potentiation of signaling by the transferase substrate, and the individual clones further characterized.

An isolated transferase protein, or a portion or fragment thereof, can be used as an immunogen to generate antibodies that bind transferase using standard techniques for polyclonal and monoclonal antibody preparation. A full-length transferase protein can be used or, alternatively, the invention provides antigenic peptide fragments of transferase for use as immunogens. The antigenic peptide of transferase comprises at least 8 amino acid residues of the amino acid sequence shown in SEQ ID NO: 2, 4, 6, 8, or 10 and encompasses an epitope of transferase such that an antibody raised against the peptide forms a specific immune complex with transferase. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of transferase that are located on the surface of the protein, e.g., hydrophilic regions, as well as regions with high antigenicity.

A transferase immunogen typically is used to prepare antibodies by immunizing a suitable subject, (e.g., rabbit, goat, mouse or other mammal) with the immunogen. An appropriate immunogenic preparation can contain, for example, recombinantly expressed transferase protein or a chemically synthesized transferase polypeptide. The preparation can further include an adjuvant, such as Freund's complete or incomplete adjuvant, or similar immunostimulatory agent. Immunization of a suitable subject with an immunogenic transferase preparation induces a polyclonal anti-transferase antibody response.

Accordingly, another aspect of the invention pertains to anti-transferase antibodies. The term “antibody” as used herein refers to immunoglobulin molecules and immunologically active portions of immunoglobulin molecules, i.e., molecules that contain an antigen binding site which specifically binds (immunoreacts with) an antigen, such as transferase. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind transferase. The term “monoclonal antibody” or “monoclonal antibody composition”, as used herein, refers to a population of antibody molecules that contain only one species of an antigen binding site capable of immunoreacting with a particular epitope of transferase. A monoclonal antibody composition thus typically displays a single binding affinity for a particular transferase protein with which it immunoreacts.

Polyclonal anti-transferase antibodies can be prepared as described above by immunizing a suitable subject with a transferase immunogen. The anti-transferase antibody titer in the immunized subject can be monitored over time by standard techniques, such as with an enzyme linked immunosorbent assay (ELISA) using immobilized transferase. If desired, the antibody molecules directed against transferase can be isolated from the mammal (e.g., from the blood) and further purified by well known techniques, such as protein A chromatography to obtain the IgG fraction. At an appropriate time after immunization, e.g., when the antitransferase antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J Immunol 127:539-46; Brown et al. (1980) J Biol. Chem .255:4980-83; Yeh et al. (1976) Proc. Natl. Acad. Sci. USA 76:2927-31; and Yeh et al. (1982) Int. J Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a transferase immunogen as described above, and the culture supernatants of the resulting hybridoma cells are screened to identify a hybridoma producing a monoclonal antibody that binds transferase.

Any of the many well known protocols used for fusing lymphocytes and immortalized cell lines can be applied for the purpose of generating an anti-transferase monoclonal antibody (see, e.g., G. Galfre et al. (1977) Nature 266:55052; Gefter et al. Somatic Cell Genet., cited supra; Lerner, Yale J Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind transferase, e.g., using a standard ELISA assay.

Alternative to preparing monoclonal antibody-secreting hybridomas, a monoclonal anti-transferase antibody can be identified and isolated by screening a recombinant combinatorial immunoglobulin library (e.g., an antibody phage display library) with transferase to thereby isolate immunoglobulin library members that bind transferase. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-transferase antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J Immunol. 141:4053-4060.

Completely human antibodies are particularly desirable for therapeutic treatment of human patients. Such antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol., 13:65-93). For a detailed discussion of this technology for producing human antibodies and human monoclonal antibodies and protocols for producing such antibodies, see e.g., U.S. Pat. No. 5,625,126; U.S. Pat. No. 5,633,425; U.S. Pat. No. 5,569,825; U.S. Pat. No. 5,661,016 and U.S. Pat. No. 5,545,806. In addition, companies such as Abgenix, Inc. (Freemont, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al. (1994) Bio/technology 12:899903).

Alternatively, an appropriate single-chain antibody (scFV) may be engineered (see, for example, Colcher, D., et al. Ann N Y Acad Sci June 1999 30;880:263-80; and Reiter, Y. Clin Cancer Res February 1996; 2(2):245-52). Such molecules contain only the Fv portion of the antibody (the portion of the antibody which specifically recognizes the antigen epitope) and none of the typical bioactive portions of the antibody. As such, they are significantly smaller in size than a regular antibody, and may conveniently be dimerized or multimerized to generate multivalent antibodies having specificities for different epitopes of the same target transferase protein.

An anti-transferase antibody (e.g., monoclonal antibody) can be used to isolate transferase by standard techniques, such as affinity chromatography or immunoprecipitation. An anti-transferase antibody can facilitate the purification of natural transferase from cells and of recombinantly produced transferase expressed in host cells. Moreover, an anti-transferase antibody can be used to detect transferase protein (e.g., in a cellular lysate or cell supernatant) in order to evaluate the abundance and pattern of expression of the transferase protein. Anti-transferase antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/ibiotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³I, ³⁵S or ³H.

III. Recombinant Expression Vectors and Host Cells

Another aspect of the invention pertains to vectors, preferably expression vectors, containing a nucleic acid encoding a transferase protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., transferase proteins, mutant forms of transferase proteins, fusion proteins, and the like).

The recombinant expression vectors of the invention can be designed for expression of transferase proteins in prokaryotic or eukaryotic cells. For example, transferase proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or nonfusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in transferase activity assays, (e.g., direct assays or competitive assays described in detail below), or to generate antibodies specific for transferase proteins, for example. In a preferred embodiment, a transferase fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gnl ). This viral polymerase is supplied by host strains BL21(DE3) or HMS174(DE3) from a resident prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the transferase expression vector is a yeast expression vector. Examples of vectors for expression in yeast S. cerevisiae include pYepSec1 (Baldari, et al., (1987) Embo J 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, transferase proteins can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufmnan et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to transferase mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a transferase nucleic acid molecule of the invention is introduced, e.g., a transferase nucleic acid molecule within a recombinant expression vector or a transferase nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a transferase protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a transferase protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a transferase protein. Accordingly, the invention further provides methods for producing a transferase protein using the host cells of the invention. In one embodiment, the method comprises culturing the host cell of the invention (into which a recombinant expression vector encoding a transferase protein has been introduced) in a suitable medium such that a transferase protein is produced. In another embodiment, the method further comprises isolating a transferase protein from the medium or the host cell.

The host cells of the invention can also be used to produce non-human transgenic animals. For example, in one embodiment, a host cell of the invention is a fertilized oocyte or an embryonic stem cell into which transferase-coding sequences have been introduced. Such host cells can then be used to create non-human transgenic animals in which exogenous transferase sequences have been introduced into their genome or homologous recombinant animals in which endogenous transferase sequences have been altered. Such animals are useful for studying the function and/or activity of a transferase protein and for identifying and/or evaluating modulators of transferase activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous transferase gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a transferase-encoding nucleic acid into the male pronuclei of a fertilized oocyte, e.g., by microinjection, retroviral infection, and allowing the oocyte to develop in a pseudopregnant female foster animal. The transferase cDNA sequence corresponding to SEQ ID NO: 1, 3, 5, 7, or 9 can be introduced as a transgene into the genome of a non-human animal. Alternatively, a non-human homologue of a human transferase gene, such as a rat or mouse transferase gene, can be used as a transgene. Alternatively, a transferase gene homologue, such as another transferase family member, can be isolated based on hybridization to the transferase corresponding to sequences of SEQ ID NOs: 1, 3, 5, 7, and 9 (described further in subsection I above) and used as a transgene. Intronic sequences and polyadenylation signals can also be included in the transgene to increase the efficiency of expression of the transgene. A tissue-specific regulatory sequence(s) can be operably linked to a transferase transgene to direct expression of a transferase protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a transferase transgene in its genome and/or expression of transferase mRNA in tissues or cells of the animals. A transgenic founder animal can then be used to breed additional animals carrying the transgene. Moreover, transgenic animals carrying a transgene encoding a transferase protein can further be bred to other transgenic animals carrying other transgenes.

To create a homologous recombinant animal, a vector is prepared which contains at least a portion of a transferase gene into which a deletion, addition or substitution has been introduced to thereby alter, e.g., functionally disrupt, the transferase gene. The transferase gene can be a human gene (e.g., the cDNA corresponding to SEQ ID NO: 1, 3, 5, 7, or 9), but more preferably, is a non-human homolog of a human transferase gene (e.g., a cDNA isolated by stringent hybridization with the nucleotide sequence of SEQ ID NO: 1, 3, 5, 7, or 9). For example, a mouse transferase gene can be used to construct a homologous recombination nucleic acid molecule, e.g., a vector, suitable for altering an endogenous transferase gene in the mouse genome. In a preferred embodiment, the homologous recombination nucleic acid molecule is designed such that, upon homologous recombination, the endogenous transferase gene is functionally disrupted (i.e., no longer encodes a functional protein; also referred to as a “knock out” vector). Alternatively, the homologous recombination nucleic acid molecule can be designed such that, upon homologous recombination, the endogenous transferase gene is mutated or otherwise altered but still encodes functional protein (e.g., the upstream regulatory region can be altered to thereby alter the expression of the endogenous transferase protein). In the homologous recombination nucleic acid molecule, the altered portion of the transferase gene is flanked at its 5′ and 3′ ends by additional nucleic acid sequence of the transferase gene to allow for homologous recombination to occur between the exogenous transferase gene carried by the homologous recombination nucleic acid molecule and an endogenous transferase gene in a cell, e.g., an embryonic stem cell. The additional flanking transferase nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced transferase gene has homologously recombined with the endogenous transferase gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The transferase nucleic acid molecules, fragments of transferase proteins, and anti-transferase antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a transferase protein or an anti-transferase antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha.-interferon, beta.interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a transferase protein of the invention has one or more of the following activities: (1) it interacts with a non-transferase protein molecule, e.g., a transferase substrate, such as a transferase receptor; (2) it activates a transferase-dependent signal transduction pathway; (3) it modulates cell proliferation, differentiation, and/or migration mechanisms; (4) it modulates angiogenesis, and, thus, can be used to, for example, (1) modulate the interaction with a non-transferase protein molecule; (2) to activate a transferase-dependent signal transduction pathway; (3) to modulate cell proliferation, differentiation, and/or migration mechanisms; (4) to modulate angiogenesis.

The isolated nucleic acid molecules of the invention can be used, for example, to express transferase protein (e.g., via a recombinant expression vector in a host cell in gene therapy applications), to detect transferase mRNA (e.g., in a biological sample) or a genetic alteration in a transferase gene, and to modulate transferase activity, as described firther below.

The transferase proteins can be used to treat disorders characterized by insufficient or excessive production of a transferase substrate or production of transferase inhibitors. In addition, the transferase proteins can be used to screen for naturally occurring transferase substrates, to screen for drugs or compounds which modulate transferase activity, as well as to treat disorders characterized by insufficient or excessive production of transferase protein or production of transferase protein forms which have decreased, aberrant or unwanted activity compared to transferase wild type protein (e.g., cell proliferation and/or differentiation disorders, such as disorders characterized by aberrant angiogenesis). Moreover, the anti-transferase antibodies of the invention can be used to detect and isolate transferase proteins, regulate the bioavailability of transferase proteins, and modulate transferase activity.

A. Screening Assays:

The invention provides methods (also referred to herein as “screening assays”) for identifying modulators, i.e., candidate or test compounds or agents (e.g., peptides, peptidomimetics, peptoids, small molecules or other drugs) which bind to transferase proteins, have a stimulatory or inhibitory effect on, for example, transferase expression or transferase activity, or have a stimulatory or inhibitory effect on, for example, the expression or activity of a transferase substrate. Compounds thus identified can be used to modulate the activity of target gene products in a therapeutic protocol, to elaborate the biological function of the target gene product, or to identify compounds that disrupt normal target gene interactions. The preferred target genes/products used in this embodiment are the transferase genes of the present invention.

Assays for the Detection of Binding Between a Test Compound and the Transferase Protein Product

In one embodiment, the invention provides assays for screening candidate or test compounds which are substrates of a transferase protein or polypeptide or biologically active portion thereof. In another embodiment, the invention provides assays for screening candidate or test compounds which bind to or modulate the activity of a transferase protein or polypeptide or biologically active portion thereof.

The test compounds of the present invention can be obtained using any of the numerous approaches in combinatorial library methods known in the art, including: biological libraries; peptoid libraries [libraries of molecules having the functionalities of peptides, but with a novel, non-peptide backbone which are resistant to enzymatic degradation but which nevertheless remain bioactive] (see, e.g., Zuckermann, R. N. et al. J Med. Chem. 1994, 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckennann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991)J Mol. Biol 222:301-310); (Ladner supra.).

In one embodiment, an assay is a cell-based assay in which a cell which expresses a transferase protein or biologically active portion thereof is contacted with a test compound and the ability of the test compound to modulate transferase activity is determined. Determining the ability of the test compound to modulate transferase activity can be accomplished by monitoring, for example, intracellular calcium and inositol 1,4,5-trisphosphate (IP3) levels, cell growth, and cell chemotaxis. The cell, for example, can be of mammalian origin, e.g., an endothelial cell.

The ability of the test compound to modulate transferase binding to a substrate or to bind to transferase can also be determined. Determining the ability of the test compound to modulate transferase binding to a substrate can be accomplished, for example, by coupling the transferase substrate with a radioisotope or enzymatic label such that binding of the transferase substrate to transferase can be determined by detecting the labeled transferase substrate in a complex. Alternatively, transferase could be coupled with a radioisotope or enzymatic label to monitor the ability of a test compound to modulate transferase binding to a transferase substrate in a complex. Determining the ability of the test compound to bind transferase can be accomplished, for example, by coupling the compound with a radioisotope or enzymatic label such that binding of the compound to transferase can be determined by detecting the labeled transferase compound in a complex. For example, compounds (e.g., transferase substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a transferase substrate) to interact with transferase without the labeling of any of the interactants. For example, a microphysiometer can be used to detect the interaction of a compound with transferase without the labeling of either the compound or the transferase. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and transferase.

In another embodiment, an assay is a cell-based assay comprising contacting a cell expressing a transferase target molecule (e.g., a transferase substrate) with a test compound and determining the ability of the test compound to modulate (e.g. stimulate or inhibit) the activity of the transferase target molecule. Determining the ability of the test compound to modulate the activity of a transferase target molecule can be accomplished, for example, by determining the ability of the transferase protein to bind to or interact with the transferase target molecule.

Determining the ability of the transferase protein or a biologically active fragment thereof, to bind to or interact with a transferase target molecule can be accomplished by one of the methods described above for determining direct binding. In a preferred embodiment, determining the ability of the transferase protein to bind to or interact with a transferase target molecule can be accomplished by determining the activity of the target molecule. For example, the activity of the target molecule can be determined by detecting induction of a cellular second messenger of the target (i.e., intracellular calcium or IP3), detecting catalytic/enzymatic activity of the target molecule upon an appropriate substrate, detecting the induction of a reporter gene (comprising a target-responsive regulatory element operatively linked to a nucleic acid encoding a detectable marker, e.g., luciferase), or detecting a target-regulated cellular response (i.e., cell growth or migration).

In yet another embodiment, an assay of the present invention is a cell-free assay in which a transferase protein or biologically active portion thereof is contacted with a test compound and the ability of the test compound to bind to the transferase protein or biologically active portion thereof is determined. Preferred biologically active portions of the transferase proteins to be used in assays of the present invention include fragments which participate in interactions with non-transferase molecules, e.g., fragments with high surface probability scores.

The cell-free assays of the present invention are amenable to use of both soluble and/or membrane-bound forms of isolated proteins (e.g., transferase proteins or biologically active portions thereof ). In the case of cell-free assays in which a membrane-bound form of an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)_{n,} 3-[(3-cholamidopropyl) dimethylamminio]-1-propane sulfonate (CHAPS), 3-[(3-cholamidopropyl)dimethylamminio]-2-hydroxy-1-propane sulfonate (CHAPSO), or N-dodecyl═N,N-dimethyl-3-ammonio-1-propane sulfonate.

The principle of the assays used to identify compounds that bind to the target gene product involves preparing a reaction mixture of the target gene protein and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring target gene product or the test substance onto a solid phase and detecting target gene product/test compound complexes anchored on the solid phase at the end of the reaction. In one embodiment of such a method, the target gene product can be anchored onto a solid surface, and the test compound, (which is not anchored), can be labeled, either directly or indirectly, with detectable labels discussed herein and which are well-known to one skilled in the art.

It is also possible to directly detect the interaction of two molecules without further sample manipulation, for example utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment of this assay method, determining the ability of the transferase protein to bind to a transferase target molecule can be accomplished without labeling either interactant using a technology such as real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). As used herein, “surface plasmon resonance” or “BIA” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In more than one embodiment of the above assay methods of the present invention, it may be desirable to immobilize either transferase or its target molecule to facilitate separation of complexed from uncomplexed forms of one or both of the proteins, as well as to accommodate automation of the assay. Binding of a test compound to a transferase protein, or interaction of a transferase protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/transferase fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or transferase protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of transferase binding or activity determined using standard techniques.

Other techniques for immobilizing proteins on matrices can also be used in the screening assays of the invention. For example, either a transferase protein or a transferase target molecule can be immobilized utilizing conjugation of biotin and streptavidin. Biotinylated transferase protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the protein-immobilized surfaces can be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with transferase protein or target molecules but which do not interfere with binding of the transferase protein to its target molecule. Such antibodies can be derivatized to the wells of the plate, and unbound target or transferase protein trapped in the wells by antibody conjugation. Methods for detecting such complexes, in addition to those described above for the GST-immobilized complexes, include immunodetection of complexes using antibodies reactive with the transferase protein or target molecule, as well as enzyme-linked assays which rely on detecting an enzymatic activity associated with the transferase protein or target molecule.

Alternatively, in another embodiment, an assay can be conducted in a liquid phase. In such an assay, the reaction products are separated from unreacted components, by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci Aug. 18, 1993(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, N. H., J Mol Recognit 1998 Winter;11(1-6):141-8; Hage, D. S., and Tweed, S. A. J Chromatogr B Biomed Sci Appl Oct. 10, 1997;699(1-2):499-525). Gel electrophoresis may also be employed to separate complexed molecules from unbound species (see, e.g., Ausubel, F. et al., eds. Current Protocols in Molecular Biology 1999, J. Wiley: New York.). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. limunoprecipitation is another common technique utilized for the isolation of a protein-protein complex from solution (see, for example, Ausubel, F. et al., eds. Current Protocols in Molecular Biology 1999, J. Wiley: New York). In this technique, all proteins binding to an antibody specific to one of the binding molecules are precipitated from solution by conjugating the antibody to a polymer bead that may be readily collected by centrifugation. The bound proteins are released from the beads (through a specific proteolysis event or other technique well known in the art which will not disturb the protein-protein interaction in the complex), and a second immunoprecipitation step is performed, this time utilizing antibodies specific for a different interacting protein. In this manner, only the complex should remain attached to the beads. The captured complex may be visualized using gel electrophoresis. The presence of a molecular complex (which may be identified by any of these techniques) indicates that a specific binding event has occurred, and that the introduced compound specifically binds to the target protein. Further, fluorescence energy transfer may also be conveniently utilized, as described herein, to detect binding without further purification of the complex from solution.

In a preferred embodiment, the assay includes contacting the transferase protein or biologically active portion thereof with a known compound which binds transferase to form an assay mixture, contacting the assay mixture with a test compound, and determining the ability of the test compound to interact with a transferase protein, wherein determining the ability of the test compound to interact with a transferase protein comprises determining the ability of the test compound to preferentially bind to transferase or biologically active portion thereof as compared to the known compound.

In yet another embodiment, the cell-free assay involves contacting a transferase protein or biologically active portion thereof with a known compound which binds the transferase protein to form an assay mixture, contacting the assay mixture with a test compound, and determining the ability of the test compound to interact with the transferase protein, wherein determining the ability of the test compound to interact with the transferase protein comprises determining the ability of the transferase protein to preferentially bind to or modulate the activity of a transferase target molecule.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, 30 peptides, and small molecules. The preferred target genes/products for use in this embodiment are the transferase genes herein identified. Towards this purpose, in an alternative embodiment, the invention provides methods for determining the ability of the test compound to modulate the activity of a transferase protein through modulation of the activity of a downstream effector of a transferase target molecule. For example, the activity of the effector molecule on an appropriate target can be determined, or the binding of the effector to an appropriate target can be determined as previously described.

The basic principle of the assay systems used to identify compounds that interfere with the interaction between the target gene product and its cellular or extracellular binding partner or partners involves preparing a reaction mixture containing the target gene product, and the binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

The assay for compounds that interfere with the interaction of the target gene products and binding partners can be conducted in a heterogeneous or homogeneous format. Heterogeneous assays involve anchoring either the target gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance; i.e., by adding the test substance to the reaction mixture prior to or simultaneously with the target gene product and interactive cellular or extracellular binding partner. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface, while the non-anchored species is labeled, either directly or indirectly. In practice, microtitre plates are conveniently utilized. The anchored species can be immobilized by non-covalent or covalent attachments. Non-covalent attachment can be accomplished simply by coating the solid surface with a solution of the target gene product or binding partner and drying. Alternatively, an immobilized antibody specific for the species to be anchored can be used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. In this approach, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-cellular or extracellular binding partner interaction can be identified.

Assays for the Detection of the Ability of a Test Compound to Modulate Expression of Transferase

In another embodiment, modulators of transferase expression are identified in a method wherein a cell is contacted with a candidate compound and the expression of transferase MRNA or protein in the cell is determined. The level of expression of transferase niRNA or protein in the presence of the candidate compound is compared to the level of expression of transferase mRNA or protein in the absence of the candidate compound. The candidate compound can then be identified as a modulator of transferase expression based on this comparison. For example, when expression of transferase mRNA or protein is greater (statistically significantly greater) in the presence of the candidate compound than in its absence, the candidate compound is identified as a stimulator of transferase mRNA or protein expression. Alternatively, when expression of transferase mRNA or protein is less (statistically significantly less) in the presence of the candidate compound than in its absence, the candidate compound is identified as an inhibitor of transferase MRNA or protein expression. The level of transferase MRNA or protein expression in the cells can be determined by methods described herein for detecting transferase MRNA or protein.

In yet another aspect of the invention, the transferase proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with transferase (“transferase-binding proteins” or “transferase-bp”) and are involved in transferase activity. Such transferase-binding proteins are also likely to be involved in the propagation of signals by the transferase proteins or transferase targets as, for example, downstream elements of a transferase-mediated signaling pathway. Alternatively, such transferase-binding proteins are likely to be transferase inhibitors.

The two-hybrid system is based on the modular nature of most transcription factors, which consist of separable DNA-binding and activation domains. Briefly, the assay utilizes two different DNA constructs. In one construct, the gene that codes for a transferase protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a transferase-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the transferase protein.

Combination Assays

In another aspect, the invention pertains to a combination of two or more of the assays described herein. For example, a modulating agent can be identified using a cell-based or a cell free assay, and the ability of the agent to modulate the activity of a transferase protein can be confirmed in vivo, e.g., in an animal such as an animal model for angiogenesis, or for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., a transferase modulating agent, an antisense transferase nucleic acid molecule, a transferase-specific antibody, or a transferase-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The choice of assay format will be based primarily on the nature and type of sensitivity/resistance protein being assayed. A skilled artisan can readily adapt protein activity assays for use in the present invention with the genes identified herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the transferase nucleotide sequences, described herein, can be used to map the location of the transferase genes on a chromosome. The mapping of the transferase sequences to chromosomes is an important first step in correlating these sequences with genes associated with disease.

Briefly, transferase genes can be mapped to chromosomes by preparing PCR primers (preferably 15-25 bp in length) from the transferase nucleotide sequences. Computer analysis of the transferase sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the transferase sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the transferase nucleotide sequences to design oligonucleotide primers, sublocalization can be achieved with panels of fragments from specific chromosomes. Other mapping strategies which can similarly be used to map a transferase sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the transferase gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The transferase sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the transferase nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The transferase nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO: 1, 3, 5, 7, or 9 can comfortably provide positive individual identification with a panel of perhaps 10 to 1,000 primers which each yield a noncoding amplified sequence of 100 bases. If predicted coding sequences, such as those in SEQ ID NOs: 1, 3, 5, 7, and 9 are used, a more appropriate number of primers for positive individual identification would be 500-2,000.

If a panel of reagents from transferase nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of Partial Transferase Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO: 1, 3, 5, 7, or 9 are particularly appropriate for this use as greater numbers of polymorphisms occur in the noncoding regions, making it easier to differentiate individuals using this technique. Examples of polynucleotide reagents include the transferase nucleotide sequences or portions thereof, e.g., fragments derived from the noncoding regions of SEQ ID NO: 1 having a length of at least 250 or 310 bases, preferably at least 270 or 330 bases; noncoding regions of SEQ ID NO: 3 having a length of at least 160 or 240 bases, preferably at least 180 or 260 bases; noncoding regions of SEQ ID NO: 5 having a length of at least 170 or 490 bases, preferably at least 190 or 510 bases; noncoding regions of SEQ ID NO: 7 having a length of at least 1370 or 2740 bases, preferably at least 1390 or 2760 bases; and noncoding regions of SEQ ID NO: 9 having a length of at least 10 or 40 bases, preferably at least 20 or 50 bases.

The transferase nucleotide sequences described herein can further be used to provide polynucleotide reagents, e.g., labeled or labelable probes which can be used in, for example, an in situ hybridization technique, to identify a specific tissue, e.g., a tissue containing endothelial cells. This can be very useful in cases where a forensic pathologist is presented with a tissue of unknown origin. Panels of such transferase probes can be used to identify tissue by species and/or by organ type.

In a similar fashion, these reagents, e.g., transferase primers or probes can be used to screen tissue culture for contamination (i.e. screen for the presence of a mixture of different types of cells in a culture).

C. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining transferase protein and/or nucleic acid expression as well as transferase activity, in the context of a biological sample (e.g., blood, serum, cells, tissue) to thereby determine whether an individual is afflicted with a disease or disorder, or is at risk of developing a disorder, associated with aberrant or unwanted transferase expression or activity. The invention also provides for prognostic (or predictive) assays for determining whether an individual is at risk of developing a disorder associated with transferase protein, nucleic acid expression or activity. For example, mutations in a transferase gene can be assayed in a biological sample. Such assays can be used for prognostic or predictive purpose to thereby prophylactically treat an individual prior to the onset of a disorder characterized by or associated with transferase protein, nucleic acid expression or activity.

As an alternative to making determinations based on the absolute expression level of selected genes, determinations may be based on the normalized expression levels of these genes. Expression levels are normalized by correcting the absolute expression level of a transferase gene by comparing its expression to the expression of a gene that is not a transferase gene, e.g., a housekeeping gene that is constitutively expressed. Suitable genes for normalization include housekeeping genes such as the actin gene. This normalization allows the comparison of the expression level in one sample, e.g., a patient sample, to another sample, e.g., a non-diseased sample, or between samples from different sources.

Alternatively, the expression level can be provided as a relative expression level. To determine a relative expression level of a gene, the level of expression of the gene is determined for 10 or more samples of different cell isolates, preferably 50 or more samples, prior to the determination of the expression level for the sample in question. The mean expression level of each of the genes assayed in the larger number of samples is determined and this is used as a baseline expression level for the gene(s) in question. The expression level of the gene determined for the test sample (absolute level of expression) is then divided by the mean expression value obtained for that gene. This provides a relative expression level and aids in identifying extreme cases of a disease.

Preferably, the samples used in the baseline determination will be from diseased or from non-diseased tissue cells. The choice of the cell source is dependent on the use of the relative expression level. Using expression found in normal tissues as a mean expression score aids in validating whether the transferase gene assayed is specific to types of cells (versus normal cells). Such a use is particularly important in identifying whether a transferase gene can serve as a target gene. In addition, as more data is accumulated, the mean expression value can be revised, providing improved relative expression values based on accumulated data. Expression data from cells provides a means for grading the severity of the disease state.

Another aspect of the invention pertains to monitoring the influence of agents (e.g., drugs, compounds) on the expression or activity of transferase in clinical trials.

These and other agents are described in further detail in the following sections.

1. Diagnostic Assays

An exemplary method for detecting the presence or absence of transferase protein or nucleic acid in a biological sample involves obtaining a biological sample from a test subject and contacting the biological sample with a compound or an agent capable of detecting transferase protein or nucleic acid (e.g., mRNA, genomic DNA) that encodes transferase protein such that the presence of transferase protein or nucleic acid is detected in the biological sample. The level of expression of the transferase gene can be measured in a number of ways, including, but not limited to: measuring the mRNA encoded by the transferase genes; measuring the amount of protein encoded by the transferase genes; or measuring the activity of the protein encoded by the transferase genes.

The level of mRNA corresponding to the transferase gene in a cell can be determined both by in situ and by in vitro formats in a biological sample using methods known in the art. The term “biological sample” is intended to include tissues, cells and biological fluids isolated from a subject, as well as tissues, cells and fluids present within a subject. A preferred biological sample is a serum sample isolated by conventional means from a subject. Many transferase expression detection methods use isolated RNA. For in vitro methods, any RNA isolation technique that does not select against the isolation of mRNA can be utilized for the purification of RNA from the cells (see, e.g., Ausubel et al., eds., 1987-1997, Current Protocols in Molecular Biology, John Wiley & Sons, Inc. New York). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full length transferase nucleic acid, such as the nucleic acid of SEQ ID NO: 1, 3, 5, 7, 9, or 11, or a portion thereof, such as an oligonucleotide of at least 7, 15, 30, 50, 100, 250 or 500 nucleotides in length and sufficient to specifically hybridize under stringent conditions to transferase mRNA or genomic DNA. Other suitable probes for use in the diagnostic assays of the invention are described herein. Hybridization of an mRNA with the probe indicates that the gene in question is being expressed.

In one format, the mRNA is immobilized on a solid surface and contacted with the probes, for example by running the isolated mRNA on an agarose gel and transferring the MRNA from the gel to a membrane, such as nitrocellulose. In an alternative format, the probes are immobilized on a solid surface and the MRNA is contacted with the probes, for example, in an Affymetrix gene chip array. A skilled artisan can readily adapt known MRNA detection methods for use in detecting the level of mRNA encoded by the transferase genes of the present invention.

An alternative method for determining the level of mRNA in a sample that is encoded by one of the transferase genes of the present invention involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers. Suitable primers for the amplification of the transferase gene are described herein.

For in situ methods, MRNA does not need to be isolated from a type of cells prior to detection. In such methods, a cell or tissue sample is prepared/processed using known histological methods. The sample is then immobilized on a support, typically a glass slide, and then contacted with a probe that can hybridize to mRNA that encodes the transferase gene being analyzed.

In another embodiment, the methods further involve obtaining a control biological sample from a control subject, contacting the control sample with a compound or agent capable of detecting transferase mRNA, or genomic DNA, such that the presence of transferase MRNA or genomic DNA is detected in the biological sample, and comparing the presence of transferase mRNA or genomic DNA in the control sample with the presence of transferase MRNA or genomic DNA in the test sample.

A variety of methods can be used to determine the level of protein encoded by one or more of the transferase genes of the present invention. In general, these methods involve the use of an agent that selectively binds to the protein, such as an antibody. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The detection methods of the invention can be used to detect transferase protein in a biological sample in vitro as well as in vivo. In vitro techniques for detection of transferase protein include enzyme linked immunosorbent assays (ELISAs), Western blots, immunoprecipitations and immunofluorescence. In vivo techniques for detection of transferase protein include introducing into a subject a labeled anti-transferase antibody. For example, the antibody can be labeled with a radioactive marker whose presence and location in a subject can be detected by standard imaging techniques.

Proteins from a type of cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether a type of cells express a protein encoded by one or more of the transferase genes of the present invention.

In one format, antibodies, or antibody fragments, can be used in methods such as Western blots or immunofluorescence techniques to detect the expressed proteins. In such uses, it is generally preferable to immobilize either the antibody or protein on a solid support. Suitable solid phase supports or carriers include any support capable of binding an antigen or an antibody. Well-known supports or carriers include glass, polystyrene, polypropylene, polyethylene, dextran, nylon, anylases, natural and modified celluloses, polyacrylamides, gabbros, and magnetite.

One skilled in the art will know many other suitable carriers for binding antibody or antigen, and will be able to adapt such support for use with the present invention. For example, protein isolated from a type of cells can be run on a polyacrylamide gel electrophoresis and immobilized onto a solid phase support such as nitrocellulose. The support can then be washed with suitable buffers followed by treatment with the detectably labeled transferase gene specific antibody. The solid phase support can then be washed with the buffer a second time to remove unbound antibody. The amount of bound label on the solid support can then be detected by conventional means.

In another embodiment, the methods further involve obtaining a control biological sample from a control subject, contacting the control sample with a compound or agent capable of detecting transferase protein, such that the presence of transferase protein is detected in the biological sample, and comparing the presence of transferase protein in the control sample with the presence of transferase protein in the test sample.

The invention also encompasses kits for detecting the presence of transferase in a biological sample. For example, the kit can comprise a compound or agent capable of detecting transferase protein or MRNA in a biological sample; means for determining the amount of transferase in the sample; and means for comparing the amount of transferase in the sample with a standard. The compound or agent can be packaged in a suitable container. The kit can further comprise instructions for using the kit to detect transferase protein or nucleic acid.

For antibody-based kits, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can comprise, for example: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

2. Prognostic Assays

The diagnostic methods described herein can furthermore be utilized to identify subjects having or at risk of developing a disease or disorder associated with aberrant or unwanted transferase expression or activity. As used herein, the term “aberrant” includes a transferase expression or activity which deviates from the wild type transferase expression or activity. Aberrant expression or activity includes increased or decreased expression or activity, as well as expression or activity which does not follow the wild type developmental pattern of expression or the subcellular pattern of expression. For example, aberrant transferase expression or activity is intended to include the cases in which a mutation in the transferase gene causes the transferase gene to be under-expressed or over-expressed and situations in which such mutations result in a non-functional transferase protein or a protein which does not function in a wild-type fashion, e.g., a protein which does not interact with a transferase substrate, e.g., a transferase receptor, or one which interacts with a non-transferase substrate. As used herein, the term “unwanted” includes an unwanted phenomenon involved in a biological response such as pain or deregulated cell proliferation. For example, the term unwanted includes a transferase expression or activity which is undesirable in a subject.

The assays described herein, such as the preceding diagnostic assays or the following assays, can be utilized to identify a subject having or at risk of developing a disorder associated with a misregulation in transferase protein activity or nucleic acid expression, such as a cell proliferation and/or differentiation disorder. Alternatively, the prognostic assays can be utilized to identify a subject having or at risk for developing a disorder associated with a misregulation in transferase protein activity or nucleic acid expression, such as a cell proliferation and/or differentiation disorder. Thus, the present invention provides a method for identifying a disease or disorder associated with aberrant or unwanted transferase expression or activity in which a test sample is obtained from a subject and transferase protein or nucleic acid (e.g., mRNA or genomic DNA) is detected, wherein the presence of transferase protein or nucleic acid is diagnostic for a subject having or at risk of developing a disease or disorder associated with aberrant or unwanted transferase expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted transferase expression or activity. For example, such methods can be used to determine whether a subject can be effectively treated with an agent for a cell proliferation and/or differentiation disorder. Thus, the present invention provides methods for determining whether a subject can be effectively treated with an agent for a disorder associated with aberrant or unwanted transferase expression or activity in which a test sample is obtained and transferase protein or nucleic acid expression or activity is detected (e.g., wherein the abundance of transferase protein or nucleic acid expression or activity is diagnostic for a subject that can be administered the agent to treat a disorder associated with aberrant or unwanted transferase expression or activity).

The methods of the invention can also be used to detect genetic alterations in a transferase gene, thereby determining if a subject with the altered gene is at risk for a disorder characterized by misregulation in transferase protein activity or nucleic acid expression, such as a cell proliferation and/or differentiation disorder. In preferred embodiments, the methods include detecting, in a sample of cells from the subject, the presence or absence of a genetic alteration characterized by at least one of an alteration affecting the integrity of a gene encoding a transferase-protein, or the mis-expression of the transferase gene. For example, such genetic alterations can be detected by ascertaining the existence of at least one of 1) a deletion of one or more nucleotides from a transferase gene; 2) an addition of one or more nucleotides to a transferase gene; 3) a substitution of one or more nucleotides of a transferase gene, 4) a chromosomal rearrangement of a transferase gene; 5) an alteration in the level of a messenger RNA transcript of a transferase gene, 6) aberrant modification of a transferase gene, such as of the methylation pattern of the genomic DNA, 7) the presence of a non-wild type splicing pattern of a messenger RNA transcript of a transferase gene, 8) a non-wild type level of a transferase-protein, 9) allelic loss of a transferase gene, and 10) inappropriate post-translational modification of a transferase-protein. As described herein, there are a large number of assays known in the art which can be used for detecting alterations in a transferase gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the transferase-gene (see Abravaya et al. (1995) Nucleic Acids Res .23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a transferase gene under conditions such that hybridization and amplification of the transferase-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a transferase gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in transferase can be identified by hybridizing a sample and control nucleic acids, e.g., DNA or RNA, to high density arrays containing hundreds or thousands of oligonucleotides probes (Cronin, M. T. et al. (1996) Human Mutation 7:244-255; Kozal, M. J. et al. (1996) Nature Medicine 2:753-759). For example, genetic mutations in transferase can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the transferase gene and detect mutations by comparing the sequence of the sample transferase with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the transferase gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type transferase sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in transferase cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a transferase sequence, e.g., a wild-type transferase sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in transferase genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control transferase nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a transferase gene.

Furthermore, any cell type or tissue in which transferase is expressed may be utilized in the prognostic assays described herein.

3. Monitoring of Effects During Clinical Trials

Monitoring the influence of agents (e.g., drugs) on the expression or activity of a transferase protein (e.g., the modulation of cell growth, differentiation, migration, and/or apoptosis mechanisms) can be applied not only in basic drug screening, but also in clinical trials. For example, the effectiveness of an agent determined by a screening assay as described herein to increase transferase gene expression, protein levels, or upregulate transferase activity, can be monitored in clinical trials of subjects exhibiting decreased transferase gene expression, protein levels, or downregulated transferase activity. Alternatively, the effectiveness of an agent determined by a screening assay to decrease transferase gene expression, protein levels, or downregulate transferase activity, can be monitored in clinical trials of subjects exhibiting increased transferase gene expression, protein levels, or upregulated transferase activity. In such clinical trials, the expression or activity of a transferase gene, and preferably, other genes that have been implicated in, for example, a transferase-associated disorder can be used as a “read out” or markers of the phenotype of a particular cell.

For example, and not by way of limitation, genes, including transferase, that are modulated in cells by treatment with an agent (e.g., compound, drug or small molecule) which modulates transferase activity (e.g., identified in a screening assay as described herein) can be identified. Thus, to study the effect of agents on transferase-associated disorders (e.g., disorders characterized by deregulated cell growth, differentiation and/or migration mechanisms), for example, in a clinical trial, cells can be isolated and RNA prepared and analyzed for the levels of expression of transferase and other genes implicated in the transferase-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of transferase or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a transferase protein, MRNA, or genomic DNA in the preadministration sample; (iii) obtaining one or more post-administration samples from the subject; (iv) detecting the level of expression or activity of the transferase protein, MRNA, or genomic DNA in the post-administration samples; (v) comparing the level of expression or activity of the transferase protein, mRNA, or genomic DNA in the pre-administration sample with the transferase protein, mRNA, or genomic DNA in the post administration sample or samples; and (vi) altering the administration of the agent to the subject accordingly. For example, increased administration of the agent may be desirable to increase the expression or activity of transferase to higher levels than detected, i.e., to increase the effectiveness of the agent. Alternatively, decreased administration of the agent may be desirable to decrease expression or activity of transferase to lower levels than detected, i.e. to decrease the effectiveness of the agent. According to such an embodiment, transferase expression or activity may be used as an indicator of the effectiveness of an agent, even in the absence of an observable phenotypic response.

4. Use of 25324, 50287, 28899, 47007, and 42967 Molecules as Surrogate Markers

The 25324, 50287, 28899, 47007, or 42967 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 25324, 50287, 28899, 47007, or 42967 molecules of the invention may be detected, and may be correlated with one or more biological states in vivo. For example, the 25324, 50287, 28899, 47007, or 42967 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35:258-264; and James (1994) AIDS Treatment News Archive 209.

The 25324, 50287, 28899, 47007, or 42967 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a 25324, 50287, 28899, 47007, or 42967 marker) transcription or expression, the amplified marker may be in a quantity which is more readily detectable than the drug itself. Also, the marker may be more easily detected due to the nature of the marker itself; for example, using the methods described herein, anti- 25324, 50287, 28899, 47007, or 42967 antibodies may be employed in an immune-based detection system for a 25324, 50287, 28899, 47007, or 42967 protein marker, or 25324, 50287, 28899, 47007, or 42967-specific radiolabeled probes may be used to detect a 25324, 50287, 28899, 47007, or 42967 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90:229-238; Schentag (1999) Am. J. Health-Syst. Pharm. 56 Suppl. 3: S21-S24; and Nicolau (1999) Am, J. Health-Syst. Pharm. 56 Suppl. 3: S16-S20.

The 25324, 50287, 28899, 47007, or 42967 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., 25324, 50287, 28899, 47007, or 42967 protein or RNA) for specific tumor markers in a subject, a drug or course of treatment may be selected that is optimized for the treatment of the specific tumor likely to be present in the subject. Similarly, the presence or absence of a specific sequence mutation in 25324, 50287, 28899, 47007, or 42967 DNA may correlate 25324, 50287, 28899, 47007, or 42967 drug response. The use of pharmacogenomic markers therefore permits the application of the most appropriate treatment for each subject without having to administer the therapy.

D. Methods of Treatment

The present invention provides for both prophylactic and therapeutic methods of treating a subject at risk of (or susceptible to) a disorder or having a disorder associated with aberrant or unwanted transferase expression or activity. With regards to both prophylactic and therapeutic methods of treatment, such treatments may be specifically tailored or modified, based on knowledge obtained from the field of pharmacogenomics. “Treatment”, as used herein, is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the transferase molecules of the present invention or transferase modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted transferase expression or activity, by administering to the subject a transferase or an agent which modulates transferase expression or at least one transferase activity. Subjects at risk for a disease which is caused or contributed to by aberrant or unwanted transferase expression or activity can be identified by, for example, any or a combination of diagnostic or prognostic assays as described herein. Administration of a prophylactic agent can occur prior to the manifestation of symptoms characteristic of the transferase aberrance, such that a disease or disorder is prevented or, alternatively, delayed in its progression. Depending on the type of transferase aberrance, for example, a transferase, transferase agonist or transferase antagonist agent can be used for treating the subject. The appropriate agent can be determined based on screening assays described herein.

2. Therapeutic Methods Treatment of a Disease by Modulation of Transferase Genes or Gene Products

A disease can be treated by negatively modulating the expression of a target gene or the activity of a target gene product. “Negative modulation,” refers to a reduction in the level and/or activity of target gene product relative to the level and/or activity of the target gene product in the absence of the modulatory treatment.

It is possible that some diseases can be caused, at least in part, by an abnormal level of gene product, or by the presence of a gene product exhibiting abnormal activity. As such, the reduction in the level and/or activity of such gene products would bring about the amelioration of the disease symptoms.

Negative Modulatory Techniques

As discussed, successful treatment of a disease can be brought about by techniques that serve to inhibit the expression or activity of target gene products.

For example, compounds, e.g., an agent identified using an assays described above, that proves to exhibit negative modulatory activity, can be used in accordance with the invention to prevent and/or ameliorate symptoms of a disease. Such molecules can include, but are not limited to peptides, phosphopeptides, small organic or inorganic molecules, or antibodies (including, for example, polyclonal, monoclonal, humanized, anti-idiotypic, chimeric or single chain antibodies, and FAb, F(ab′)₂ and FAb expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity.

Among the compounds that can exhibit the ability to prevent and/or ameliorate symptoms of a disease are antisense, ribozyme, and triple helix molecules. Such molecules can be designed to reduce or inhibit either wild type, or if appropriate, mutant target gene activity. Techniques for the production and use of such molecules are well known to those of skill in the art.

Anti-sense RNA and DNA molecules act to directly block the translation of MRNA by hybridizing to targeted mRNA and preventing protein translation. With respect to antisense DNA, oligodeoxyribonucleotides derived from the translation initiation site, e.g., between the −10 and +10 regions of the target gene nucleotide sequence of interest, are preferred.

Ribozymes are enzymatic RNA molecules capable of catalyzing the specific cleavage of RNA. (For a review, see, for example, Rossi, 1994, Current Biology 4:469-471.) The mechanism of ribozyme action involves sequence specific hybridization of the ribozyme molecule to complementary target RNA, followed by an endonucleolytic cleavage. The composition of ribozyme molecules must include one or more sequences complementary to the target gene MRNA and must include the well-known catalytic sequence responsible for mRNA cleavage. For this sequence, see U.S. Pat. No. 5,093,246, that is incorporated by reference herein in its entirety. As such within the scope of the invention are engineered hammerhead motif ribozyme molecules that specifically and efficiently catalyze endonucleolytic cleavage of RNA sequences encoding target gene proteins.

Specific ribozyme cleavage sites within any potential RNA target are initially identified by scanning the molecule of interest for ribozyme cleavage sites that include the following sequences, GUA, GUU, and GUC. Once identified, short RNA sequences of between 15 and 20 ribonucleotides corresponding to the region of the target gene containing the cleavage site can be evaluated for predicted structural features, such as secondary structure, that can render the oligonucleotide sequence unsuitable. The suitability of candidate sequences can also be evaluated by testing their accessibility to hybridization with complementary oligonucleotides, using ribonuclease protection assays.

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, that generally require sizeable stretches of either purines or pyrimidines to be present on one strand of a duplex. Nucleotide sequences can be pyrimidinebased, that will result in TAT and CGC⁺ triplets across the three associated strands of the resulting triple helix. The pyrimidine-rich molecules provide base complementarily to a purine-rich region of a single strand of the duplex in a parallel orientation to that strand. In addition, nucleic acid molecules can be chosen that are purine-rich, for example, contain a stretch of G residues. These molecules will form a triple helix with a DNA duplex that is rich in GC pairs, in that the majority of the purine residues are located on a single strand of the targeted duplex, resulting in GGC triplets across the three strands in the triplex.

Alternatively, the potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to reduce or inhibit mutant gene expression, it is possible that the technique utilized can also efficiently reduce or inhibit the transcription (triple helix) and/or translation (antisense, ribozyme) of mRNA produced by normal target gene alleles such that the possibility can arise wherein the concentration of normal target gene product present can be lower than is necessary for a normal phenotype. In such cases, to ensure that substantially normal levels of target gene activity are maintained, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Anti-sense RNA and DNA, nibozyme and triple helix molecules of the invention can be prepared by any method known in the art for the synthesis of DNA and RNA molecules. These include techniques for chemically synthesizing oligodeoxyribonucleotides and oligoribonucleotides well known in the art such as, for example, solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules can be introduced as a means of increasing intracellular stability and half-life. Possible modifications include but are not limited to the addition of flanking sequences of ribo- or deoxy- nucleotides to the 5′ and/or 3′ ends of the molecule or the use of phosphorothioate or 2′ O-methyl rather than phosphodiesterase linkages within the oligodeoxyribonucleotides backbone.

Another method by which nucleic acid molecules may be utilized in treatment or prevention of a disease state characterized by transferase expression is through the use of aptamer molecules specific for transferase protein. Aptamers are nucleic acid molecules having a tertiary structure which permits them to specifically bind to protein ligands (see, e.g., Osborne, et al. Curr. Opin. Chem Biol. 1997, 1(1): 5-9; and Patel, D. J. Curr Opin Chem Biol 1997 Jun; 1(1):32-46). Since nucleic acid molecules may in many cases be more conveniently introduced into target cells than therapeutic protein molecules may be, aptamers offer a method by which transferase protein activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of a disease. Antibodies can be generated using standard techniques against the proteins themselves or against peptides corresponding to portions of the proteins. The antibodies include but are not limited to polyclonal, monoclonal, Fab fragments, single chain antibodies, scFV molecules, chimeric antibodies, and the like, as described herein.

In circumstances wherein injection of an animal or a human subject with a transferase protein or epitope for the purpose of stimulating antibody production is harmfuil to the subject, due to the nature of the transferase protein or portion thereof, it is possible to generate an immune response against transferase through the use of anti-idiotypic antibodies (see, for example, Herlyn, D. Ann Med 1999;31(1):66-78; and Bhattacharya-Chatteijee, M., and Foon, K. A. Cancer Treat Res 1998;94:51-68). Anti-idiotypic antibodies are antibodies which specifically recognize the antigen-binding portion of another antibody, and as such, their antigen-binding domain should be nearly identical in structure to an epitope of the antigen to which the first antibody was specific. For example, an anti-idiotypic antibody specific for the antigen-binding domain of an anti-transferase antibody should have an antigen-binding domain structure similar to that of some portion of the transferase protein. If such an anti-idiotypic antibody is introduced into a mammal or human subject, it should stimulate the production of anti-anti-idiotypic antibodies, which should be specific to the transferase protein. Vaccines directed to a disease state characterized by transferase expression may also be generated in this fashion.

In instances where the target gene protein to that the antibody is directed to is intracellular and whole antibodies are used, internalizing antibodies may be preferred. However, lipofectin or liposomes can be used to deliver the antibody or a fragment of the Fab region that binds to the target gene epitope into cells. Where fragments of the antibody are used, the smallest inhibitory fragment that binds to the target protein's binding domain is preferred. For example, peptides having an amino acid sequence corresponding to the domain of the variable region of the antibody that binds to the target gene protein can be used. Such peptides can be synthesized chemically or produced via recombinant DNA technology using methods well known in the art (e.g., see Creighton, 1983, supra; and Sambrook et al., 1989, supra). Alternatively, single chain neutralizing antibodies that bind to intracellular target gene product epitopes can also be administered. Such single chain antibodies can be administered, for example, by expressing nucleotide sequences encoding single-chain antibodies within the target cell population by utilizing, for example, techniques such as those described in Marasco et al. (1993, Proc. Natl. Acad. Sci. USA 90:7889-7893).

Therapeutic Treatment

The identified compounds that inhibit target gene expression, synthesis and/or activity can be administered to a patient at therapeutically effective doses to prevent, treat or ameliorate a disease. A therapeutically effective dose refers to that amount of the compound sufficient to result in amelioration of symptoms of a disease.

Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate transferase activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell, R. J. et al (1996) Current Opinion in Biotechnology 7:89-94 and in Shea, K. J. (1994) Trends in Polymer Science 2:166-173.

Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, G. et al (1993) Nature 361:645-647. Through the use of isotope-labeling, the “free” concentration of compound which modulates the expression or activity of transferase can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. An rudimentary example of such a “biosensor” is discussed in Kriz, D. et al. (1995) Analytical Chemistry 67:2142-2144.

Another aspect of the invention pertains to methods of modulating transferase expression or activity for therapeutic purposes. Accordingly, in an exemplary embodiment, the modulatory method of the invention involves contacting a cell with a transferase or agent that modulates one or more of the activities of transferase protein activity associated with the cell. An agent that modulates transferase protein activity can be an agent as described herein, such as a nucleic acid or a protein, a naturally-occurring target molecule of a transferase protein (e.g., a transferase substrate or receptor), a transferase antibody, a transferase agonist or antagonist, a peptidomimetic of a transferase agonist or antagonist, or other small molecule. In one embodiment, the agent stimulates one or more transferase activities. Examples of such stimulatory agents include active transferase protein and a nucleic acid molecule encoding transferase that has been introduced into the cell. In another embodiment, the agent inhibits one or more transferase activities. Examples of such inhibitory agents include antisense transferase nucleic acid molecules, anti-transferase antibodies, and transferase inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a transferase protein or nucleic acid molecule. In one embodiment, the method involves administering an agent (e.g., an agent identified by a screening assay described herein), or combination of agents that modulates (e.g., upregulates or downregulates) transferase expression or activity. In another embodiment, the method involves administering a transferase protein or nucleic acid molecule as therapy to compensate for reduced, aberrant, or unwanted transferase expression or activity.

Stimulation of transferase activity is desirable in situations in which transferase is abnormally downregulated and/or in which increased transferase activity is likely to have a beneficial effect. For example, stimulation of transferase activity is desirable in situations in which a transferase is downregulated and/or in which increased transferase activity is likely to have a beneficial effect. Likewise, inhibition of transferase activity is desirable in situations in which transferase is abnormally upregulated and/or in which decreased transferase activity is likely to have a beneficial effect.

3. Pharnacogenomics

The transferase molecules of the present invention, as well as agents, or modulators which have a stimulatory or inhibitory effect on transferase activity (e.g., transferase gene expression) as identified by a screening assay described herein can be administered to individuals to treat (prophylactically or therapeutically) transferase-associated disorders (e.g., cell proliferation and/or differentiation disorders, or disorders characterized by aberrant angiogenesis) associated with aberrant or unwanted transferase activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a transferase molecule or transferase modulator as well as tailoring the dosage and/or therapeutic regimen of treatment with a transferase molecule or transferase modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6D) is a common inherited enzymopathy in which the main clinical complication is haemolysis after ingestion of oxidant drugs (anti-malarials, sulfonamides, analgesics, nitrofurans) and consumption of fava beans.

One pharnacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some tenmillion known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., a transferase protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a transferase molecule or transferase modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a transferase molecule or transferase modulator, such as a modulator identified by one of the exemplary screening assays described herein.

The present invention further provides methods for identifying new anti-disease agents, or combinations, that are based on identifying agents that modulate the activity of one or more of the gene products encoded by one or more of the transferase genes of the present invention, wherein these products may be associated with resistance of the cells to a therapeutic agent. Specifically, the activity of the proteins encoded by the transferase genes of the present invention can be used as a basis for identifying agents for overcoming agent resistance. By blocking the activity of one or more of the resistance proteins, cells will become sensitive to treatment with an agent that the unmodified cells were resistant to.

### EXAMPLES

### Example 1

Identification and Characterization of Human 25324, 50287, 28899, 47007, or 42967 cDNAs

The human 25324 sequence (FIG. 1A-B; SEQ ID NO: 1), which is approximately 1892 nucleotides long including untranslated regions, contains a predicted methionine-initiated coding sequence of about 1275 nucleotides (nucleotides 1-1275 of SEQ ID NO: 11). The coding sequence encodes a 425 amino acid protein (SEQ ID NO: 2).

The human 50287 sequence (FIG. 4; SEQ ID NO: 3), which is approximately 1892 nucleotides long including untranslated regions, contains a predicted methionine-initiated coding sequence of about 552 nucleotides (1-552 of SEQ ID NO: 12). The coding sequence encodes a 184 amino acid protein (SEQ ID NO: 4).

The human 28899 sequence (FIG. 7A-B; SEQ ID NO: 5), which is approximately 1832 nucleotides long including untranslated regions, contains a predicted methionine-initiated coding sequence of about 1995 nucleotides (nucleotides 1-1128 of SEQ ID NO: 13). The coding sequence encodes a 376 amino acid protein (SEQ ID NO: 6).

The human 47007 sequence (FIG. 10A-C; SEQ ID NO: 7), which is approximately 5426 nucleotides long including untranslated regions, contains a predicted methionine-initiated coding sequence of about 1269 nucleotides (1-1269 of SEQ ID NO: 14). The coding sequence encodes a 423 amino acid protein (SEQ ID NO: 8).

The human 42967 sequence (FIG. 13; SEQ ID NO: 9), which is approximately 602 nucleotides long including untranslated regions, contains a predicted methionine-initiated coding sequence of about 519 nucleotides (nucleotides 1-519 of SEQ ID NO: 3). The coding sequence encodes a 173 amino acid protein (SEQ ID NO: 15).

### Example 2

Expression and Tissue Distribution of 25324, 50287, 28899, 47007, or 42967 mRNA

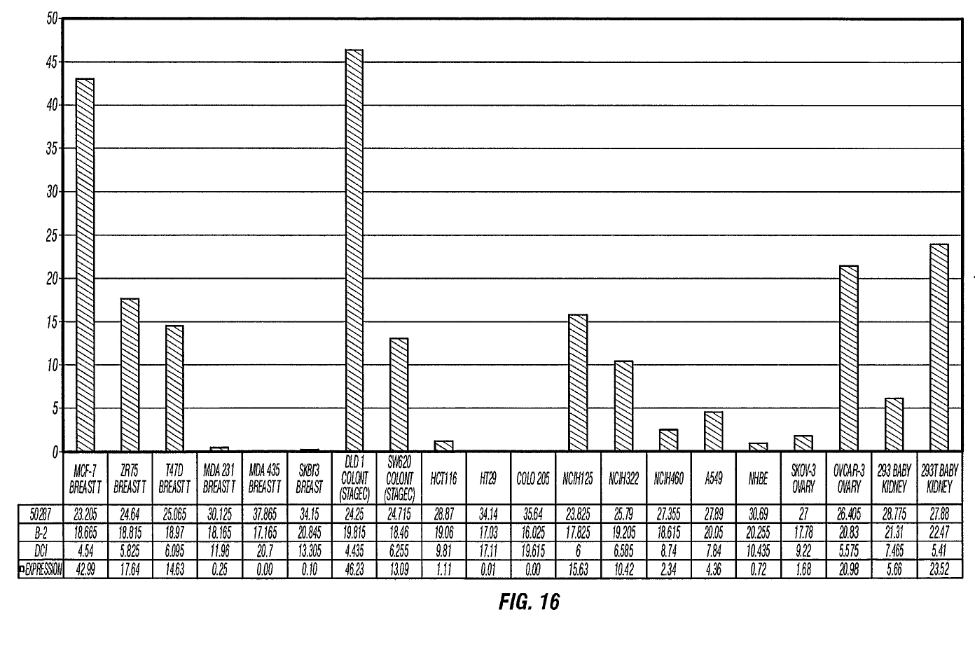

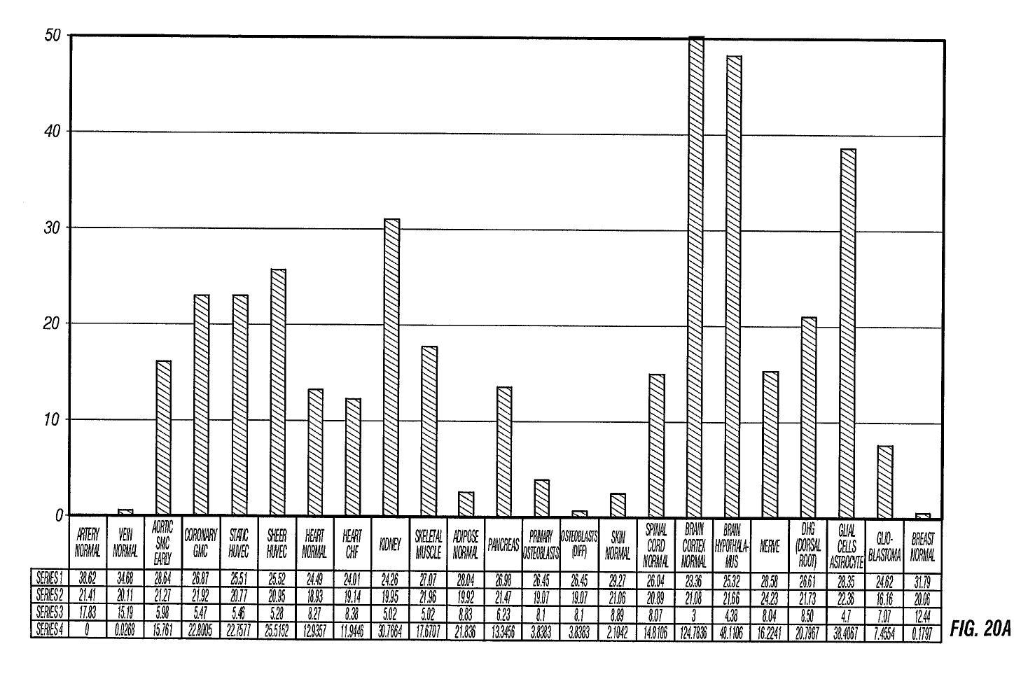

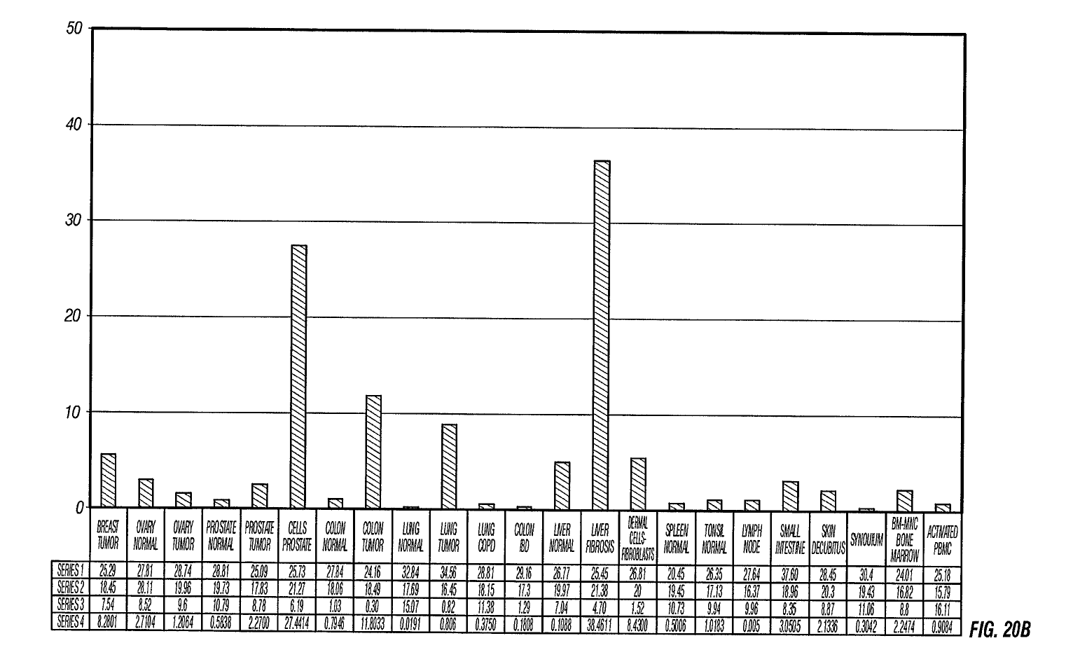

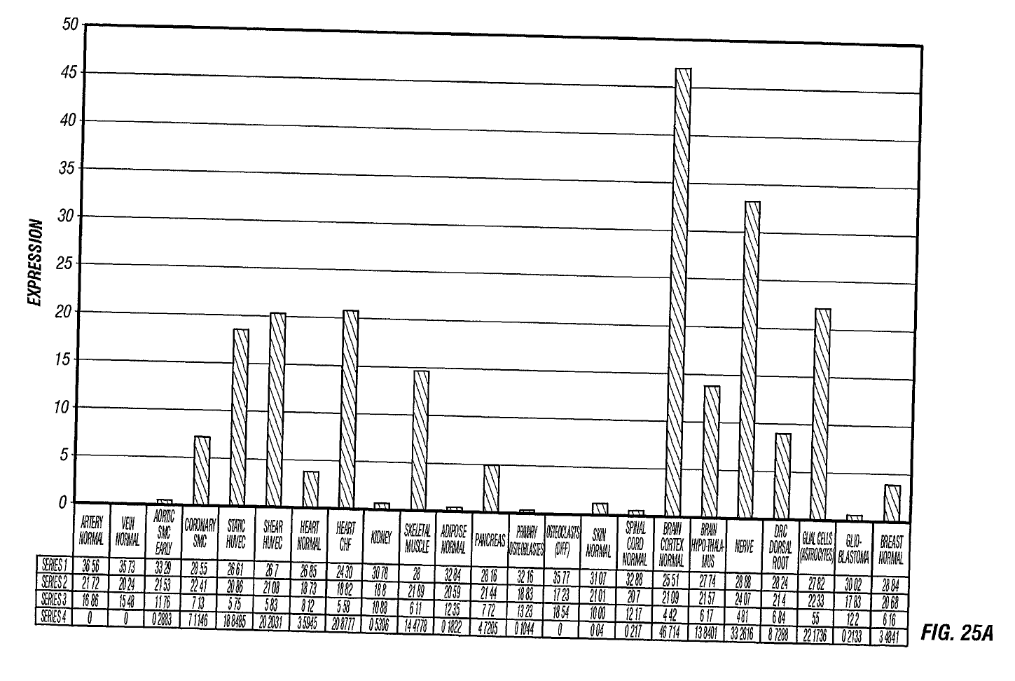

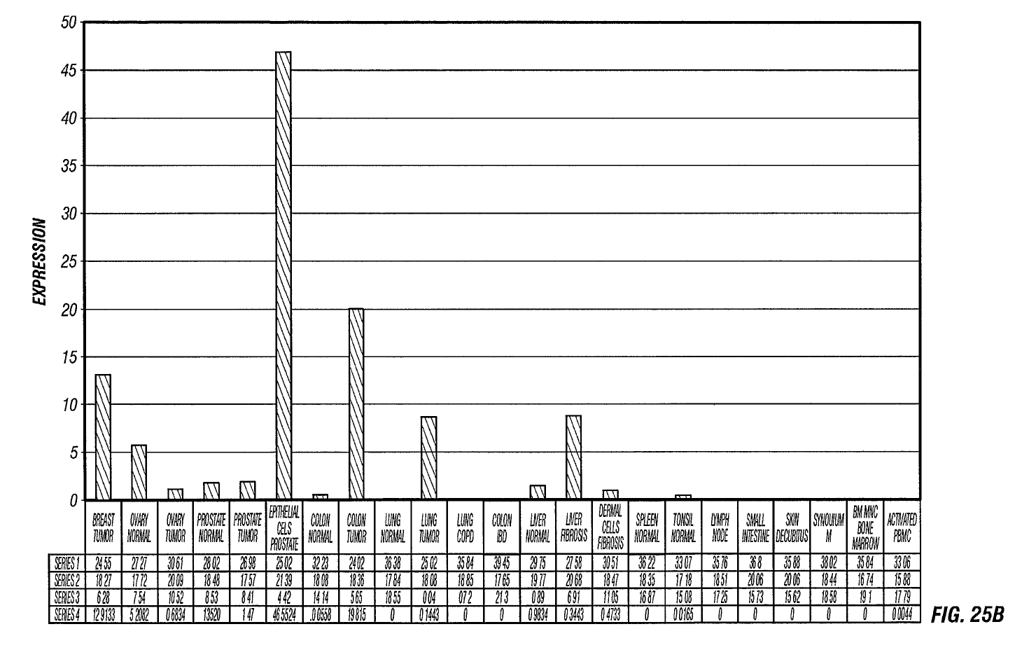

Northern blot hybridizations with various RNA samples can be performed under standard conditions and washed under stringent conditions, i.e., 0.2×SSC at 65° C. A DNA probe corresponding to all or a portion of the 25324, 50287, 28899, 47007, or 42967 cDNA (SEQ ID NOs: 1, 3, 5, 7, or 9) can be used. The DNA is radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb hybridization solution (Clontech) and washed at high stringency according to manufacturer's recommendations. TaqMan real-time quantitative RT-PCR is used to detect the presence of RNA transcript corresponding to human 25324, 50287, 28899, 47007, or 42967 in several tissues. It is found that the corresponding orthologs of 25324, 50287, 28899, 47007, or 42967 are expressed in a variety of tissues. The results of the screening for 50287, 28899, and 47007, are shown in FIGS. 16-26.

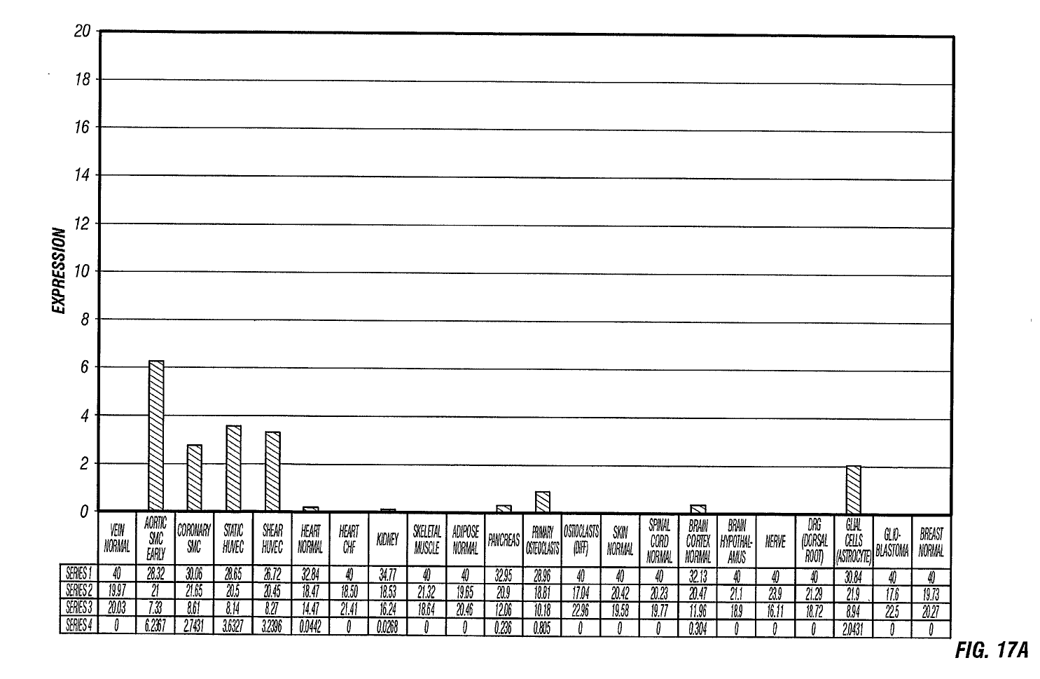

Reverse Transcriptase PCR (RT-PCR) was used to detect the presence of RNA transcript corresponding to human 50287, 28899, or 47007 in RNA prepared from tumor and normal tissues. FIGS. 17, 20 and 25 illustrate the relative expression levels and tissue distribution of the 50287, 28899, and 47007 genes in various tissues using Taq Man PCR. If a subject has a disease characterized by underexpression or overexpression of a 50287, 28899, or 47007 gene, modulators which have a stimulatory or inhibitory effect on transferase activity (e.g., transferase gene expression) can be administered to individuals to treat (prophylactically or therapeutically) transferase-associated disorders.

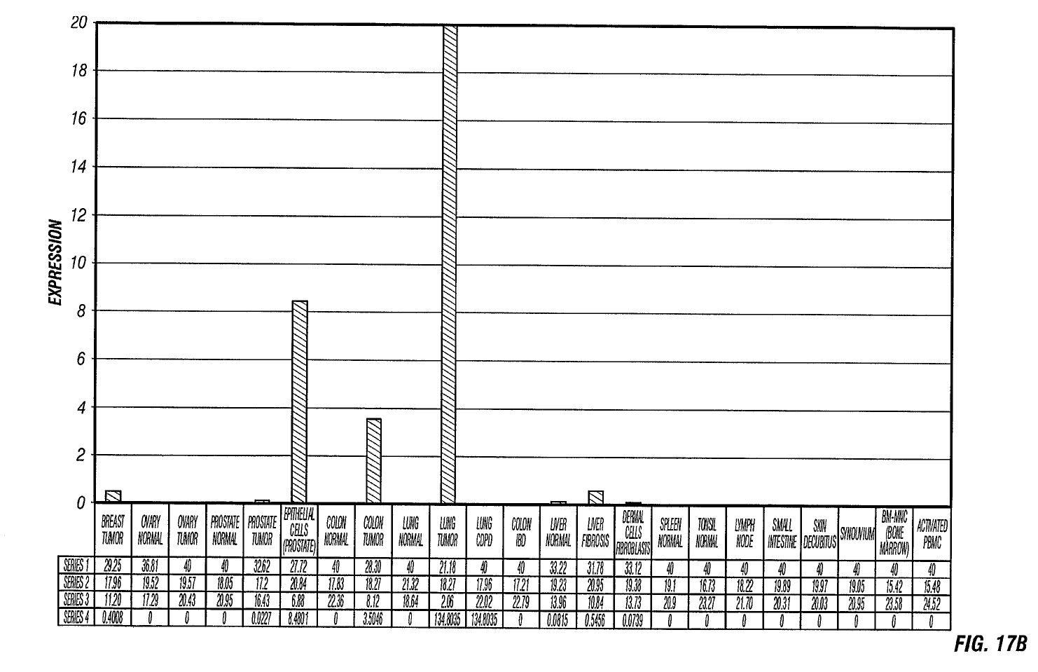

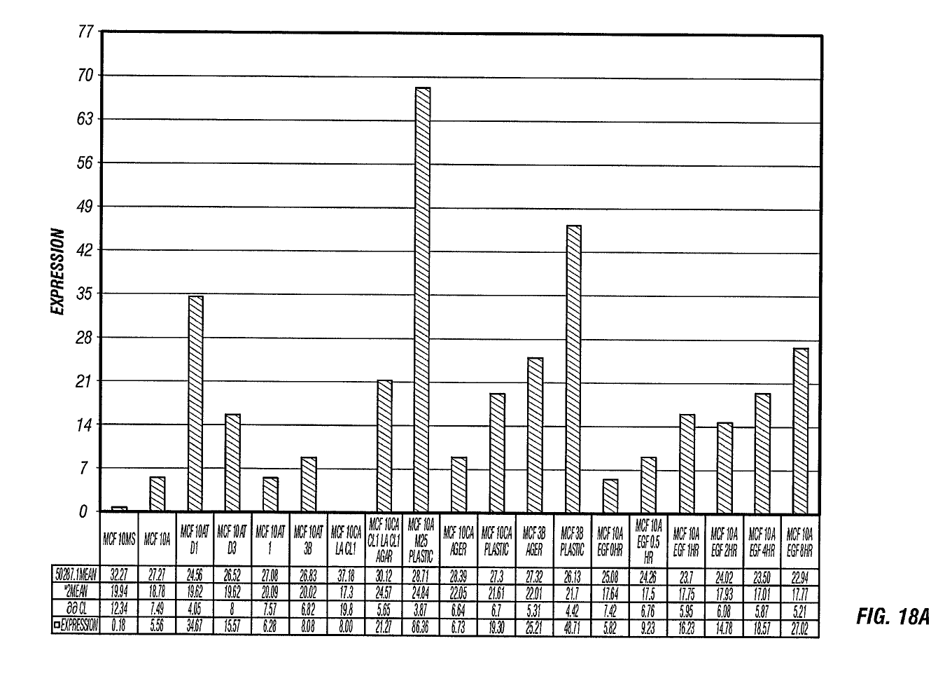

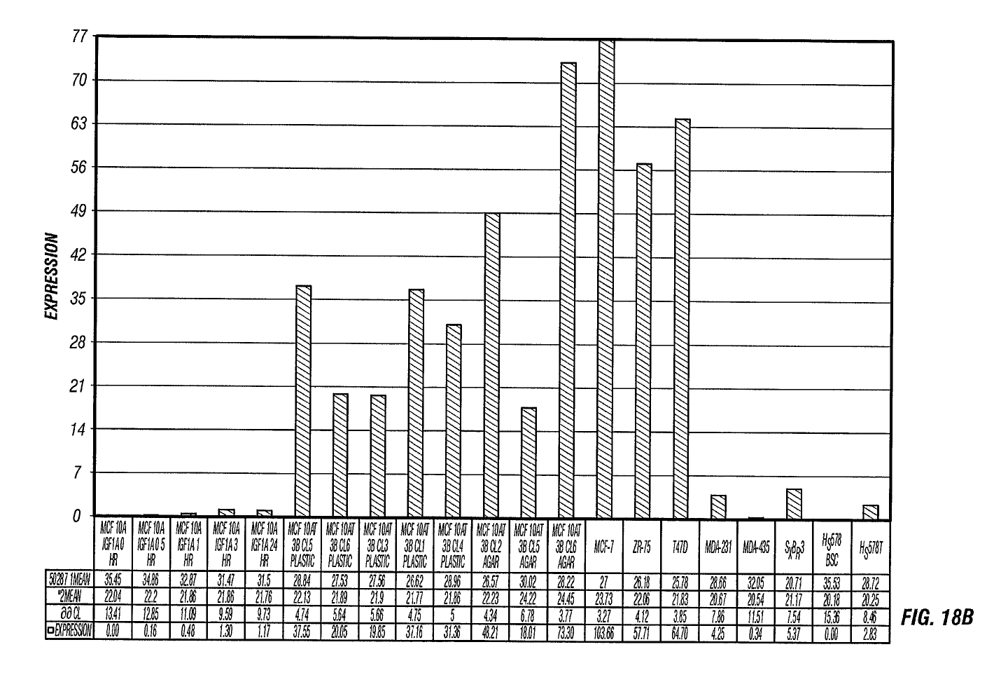

Variable expression was found in xenographs of cell lines tested as shown in FIG. 16 for 50287, and the highest expression was found in MCF-7 breast tumor cell line and the DLD1 colon tumor cell line. In addition, the 50287 gene was highly expressed in lung tumor as shown in FIG. 17. A panel as shown in FIG. 18 of human normal breast cell lines and breast carcinoma cells detected using real-time quantitative RT-PCR Taq Man analysis shows that the highest level of expression was found in the MCF-7 breast carcinoma cells.

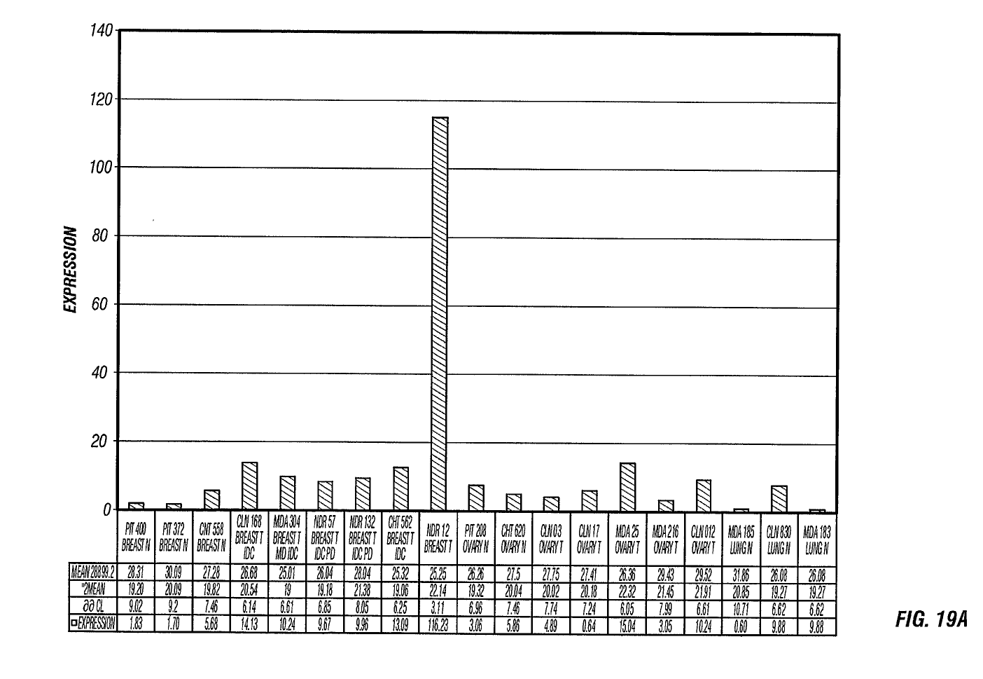

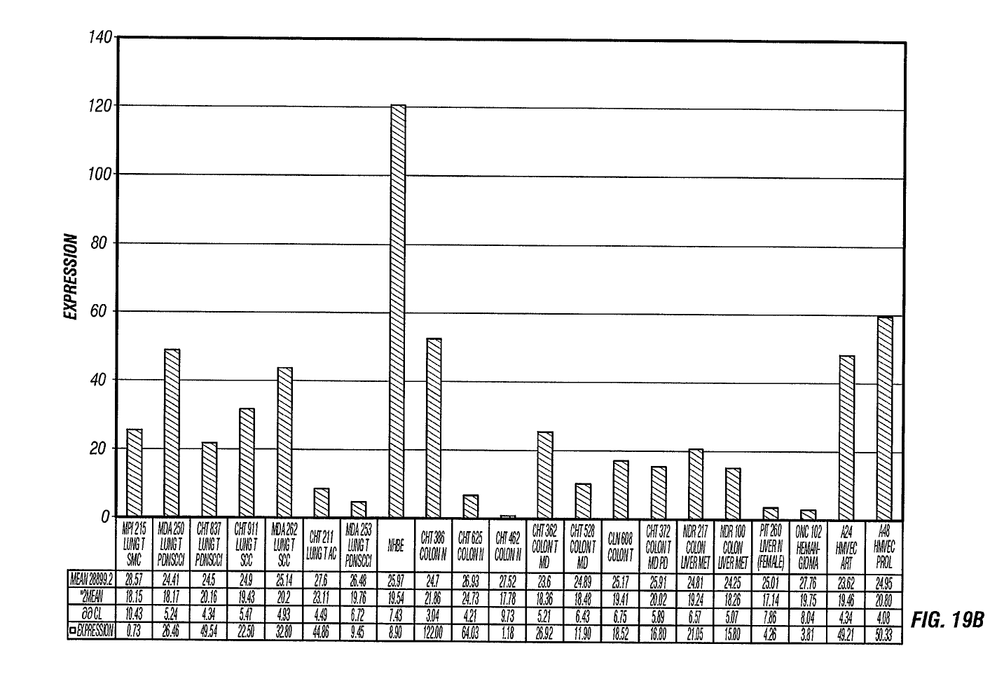

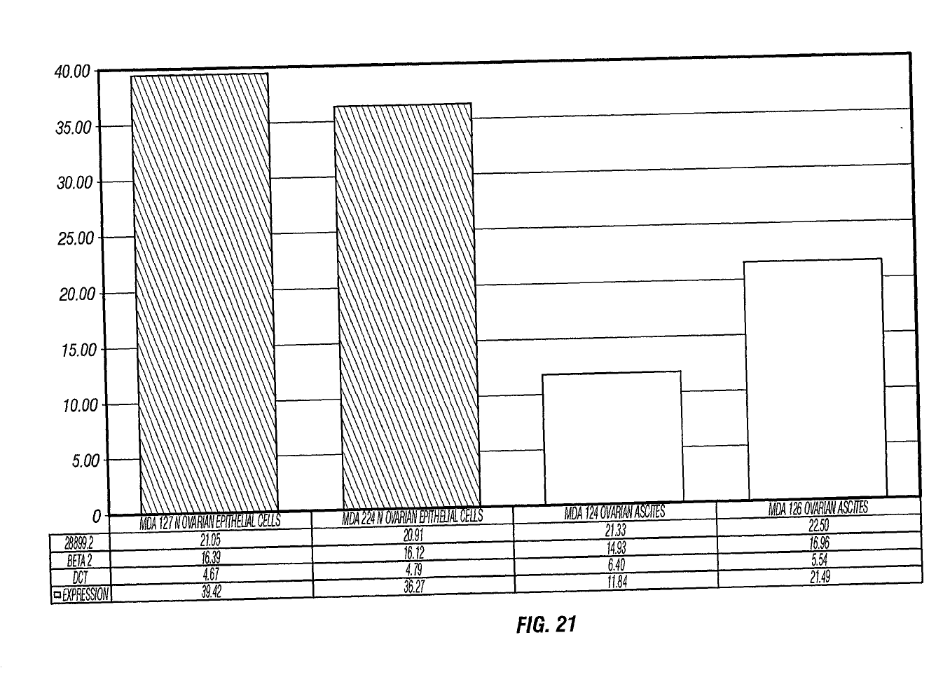

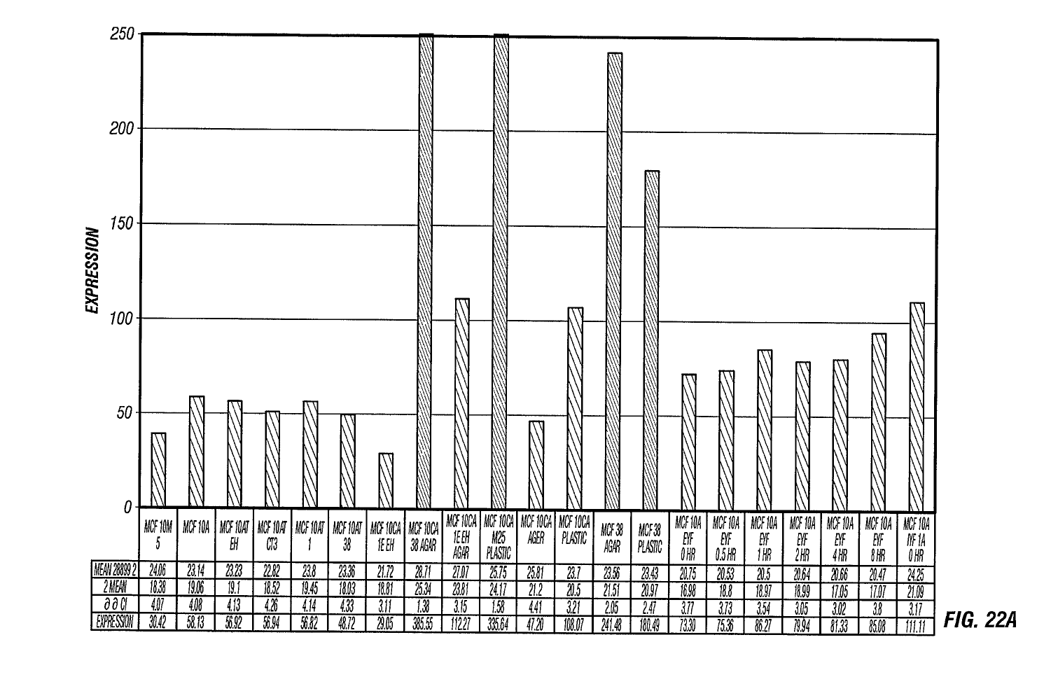

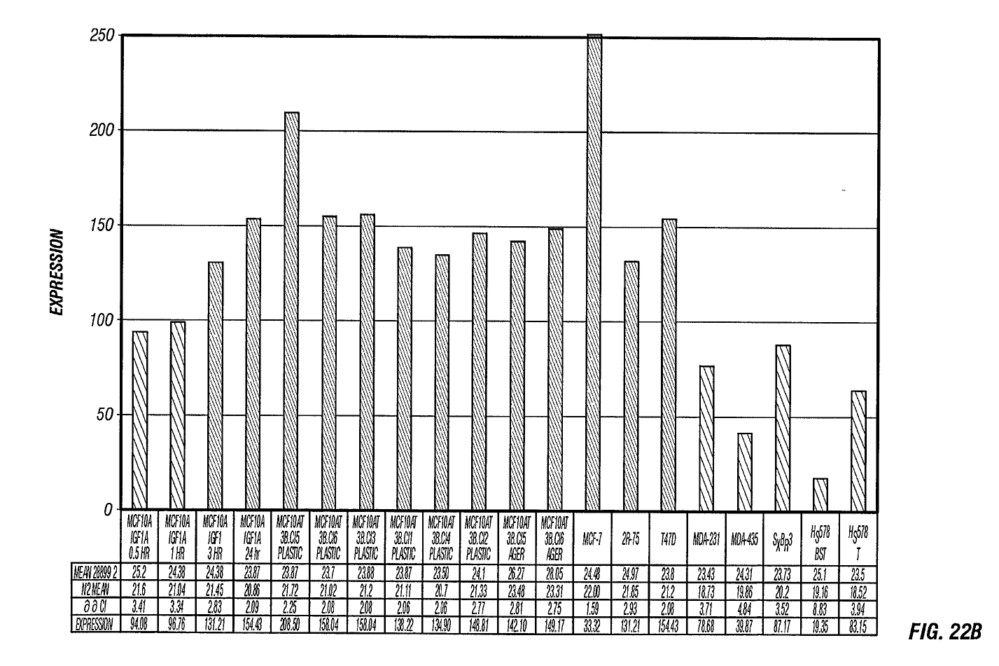

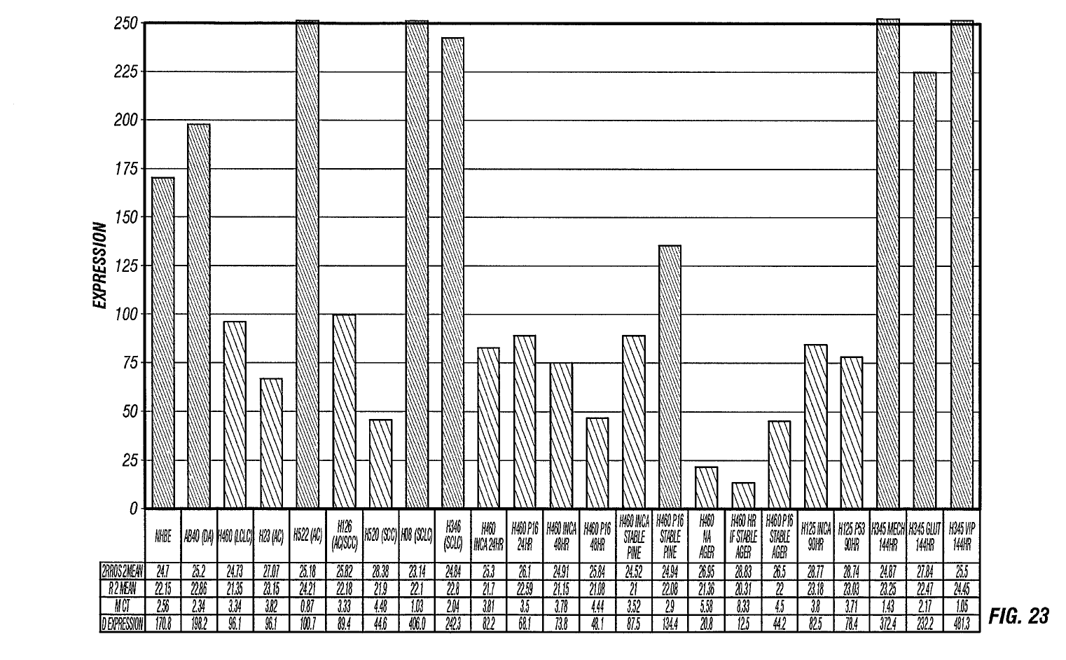

With regard to 28899, FIG. 19 shows an increased expression in 6/6 breast tumor samples in comparison with normal breast tissue; 2/4 ovary tumor samples in comparison with normal ovary tissue; and 5/7 various lung tumor samples in comparison with normal lung tissue, which expression was detected using Taq Man analysis. Further results shown in FIG. 22 relating to expression in breast cell lines show the highest level of expression in the MCF-10AT 3B and MCF-10A m25 cells and MCF-7 breast carcinoma cells. Additional data for ovarian cell lines show the highest level of expression in the MDA 127 N ovarian epithelial cells as shown in FIG. 21. Lung cell line results are shown in FIG. 23, which shows the highest level of expression in H522 (AC), H69 (SCLC), H345 Mock, and H345 VIP cancer cell lines.

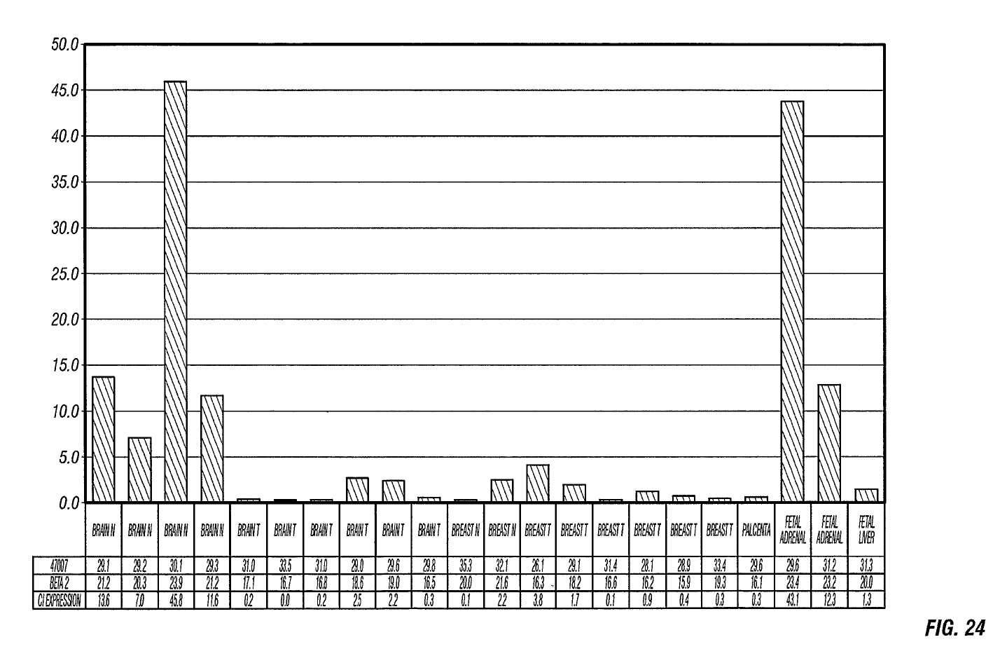

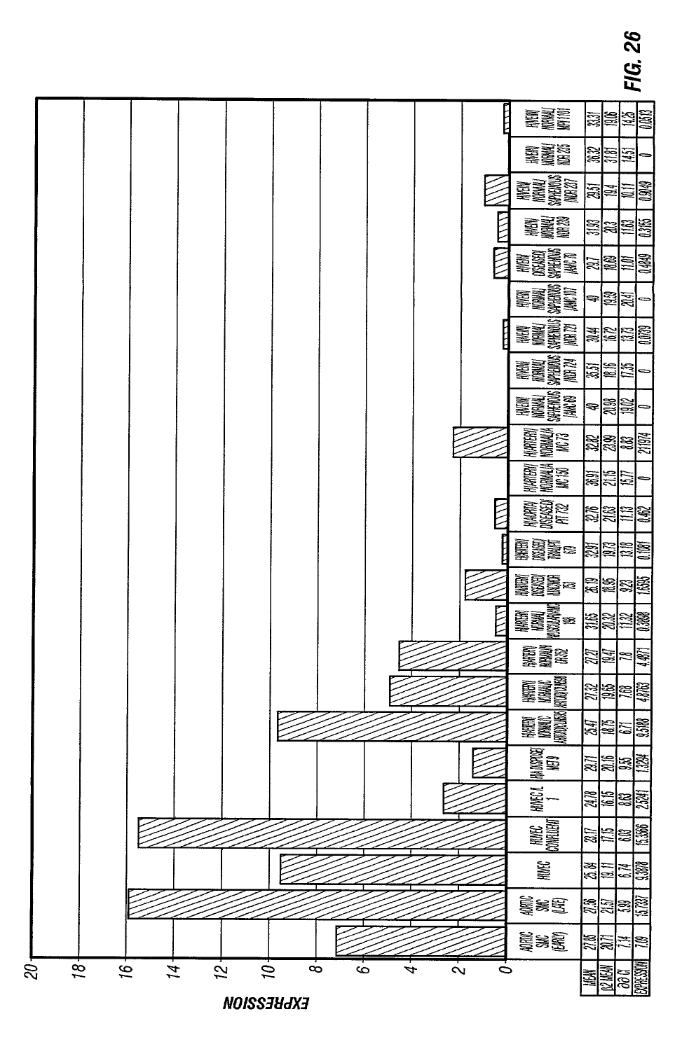

In an angiogenic panel, the results of which are shown in FIG. 24, decreased 47007 expression is shown in 6/6 brain tumor samples in comparison with normal brain tissue; and showing high expression in fetal adrenal tissues. In a panel of human normal and diseased blood vessels, as shown in FIG. 26, the highest level of 47007 expression was found in aortic smooth muscle cells (SMC) (late) and confluent human umbilical vein epithelial cells (HUVEC).

As seen by these results, 50287, 28899, or 47007 molecules have been found to be overexpressed or underexpressed in some tumor or cells involved in angiogenic processes, where the molecules may be inappropriately propagating either cell proliferation or cell survival signals or have aberrant transferase activity associated with aberrant angiogenesis. As such, 50287, 28899, or 47007 molecules may serve as specific and novel identifiers of such tumor cells or disorders involving aberrant angiogenesis. Further, inhibitors of the 50287 molecules are also useful for the treatment of cancer, preferably breast, lung and colon cancer, and useful as a diagnostic. In addition, inhibitors of the 28899, molecules are also useful for the treatment of cancer, preferably breast, ovarian and lung cancer, and useful as a diagnostic. Modulators of 47007 molecules are also useful for the treatment of disorders involving aberrant angiogenesis, for example stimulators of the 47007 molecules are useful for the treatment of tumor angiogenesis, preferably brain tumor angiogenesis, as well as the treatment of cardiovascular disorders and inflammation.

### Example 3

Recombinant Expression of 25324, 50287, 28899, 47007, or 42967 in Bacterial Cells

In this example, 25324, 50287, 28899, 47007, or 42967 is expressed as a recombinant glutathione-S-transferase (GST) fusion polypeptide in E. coli and the fusion polypeptide is isolated and characterized. Specifically, 25324, 50287, 28899, 47007, or 42967 is fused to GST and this fusion polypeptide is expressed in E. coli, e.g., strain PEB199. Expression of the GST-25324, 50287, 28899, 47007, or 42967 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 4

Expression of Recombinant 25324, 50287, 28899, 47007, or 42967 Protein in COS Cells

To express the 25324, 50287, 28899, 47007, or 42967 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 25324, 50287, 28899, 47007, or 42967 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 25324, 50287, 28899, 47007, or 42967 DNA sequence is amplified by PCR using two primers. The 5′ primer contains the restriction site of interest followed by approximately twenty nucleotides of the 25324, 50287, 28899, 47007, or 42967 coding sequence starting from the initiation codon; the 3′ end sequence contains complementary sequences to the other restriction site of interest, a translation stop codon, the HA tag or FLAG tag and the last 20 nucleotides of the 25324, 50287, 28899, 47007, or 42967 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the 25324, 50287, 28899, 47007, or 42967 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 25324, 50287, 28899, 47007, or 42967-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the 25324, 50287, 28899, 47007, or 42967 polypeptide is detected by radiolabelling (³⁵S-methionine or ³⁵S-cysteine available from NEN, Boston, Mass., can be used) and immunoprecipitation (Harlow, E. and Lane, D. Antibodies. A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 25324, 50287, 28899, 47007, or 42967 coding sequence is cloned directly into the polylinker of the pCDNA/Amp vector using the appropriate restriction sites. The resulting plasmid is transfected into COS cells in the manner described above, and the expression of the 25324, 50287, 28899, 47007, or 42967 polypeptide is detected by radiolabelling and immunoprecipitation using a 25324, 50287, 28899, 47007, or 42967 specific monoclonal antibody.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. An isolated 25324, 50287, 28899, 47007, or 42967 nucleic acid molecule selected from the group consisting of:
a) a nucleic acid molecule comprising a nucleotide sequence which is at least 60% identical to the nucleotide sequence of SEQ ID NO: 1, SEQ ID NO: 3, SEQ ID NO: 5, SEQ ID NO: 7, SEQ ID NO: 9, SEQ ID NO: 1, SEQ ID NO: 12, SEQ ID NO: 13, SEQ ID NO: 14, SEQ ID NO: 15, or the nucleotide sequence of the DNA insert of the plasmid deposited with ATCC as Accession Number______;
b) a nucleic acid molecule comprising a fragment of at least 15 nucleotides of the nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, or the nucleotide sequence of the DNA insert of the plasmid deposited with ATCC as Accession Number______;
c) a nucleic acid molecule which encodes a polypeptide comprising the amino acid sequence of SEQ ID NO: 2, SEQ ID NO: 4, SEQ ID NO: 6, SEQ ID NO: 8, SEQ ID NO: 10, or the amino acid sequence encoded by the cDNA insert of the plasmid deposited with the ATCC as Accession Number______;
d) a nucleic acid molecule which encodes a fragment of a polypeptide comprising the amino acid sequence of SEQ ID NO: 2, SEQ ID NO: 4, SEQ ID NO: 6, SEQ ID NO: 8, SEQ ID NO: 10, or the amino acid sequence encoded by the cDNA insert of the plasmid deposited with the ATCC as Accession Number______, wherein the fragment comprises at least 15 contiguous amino acids of SEQ ID NO: 2, SEQ ID NO: 4, SEQ ID NO: 6, SEQ ID NO: 8, SEQ ID NO: 10, or the amino acid sequence encoded by the cDNA insert of the plasmid deposited with the ATCC as Accession Number______;
e) a nucleic acid molecule which encodes a naturally occurring allelic variant of a polypeptide comprising the amino acid sequence of SEQ ID NO: 2, SEQ ID NO: 4, SEQ ID NO: 6, SEQ ID NO: 8, SEQ ID NO: 10, or the amino acid sequence encoded by the cDNA insert of the plasmid deposited with the ATCC as Accession Number______, wherein the nucleic acid molecule hybridizes to a nucleic acid molecule comprising SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, or a complement thereof, under stringent conditions;
f) a nucleic acid molecule comprising the nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, or the nucleotide sequence of the DNA insert of the plasmid deposited with ATCC as Accession Number______; and
g) a nucleic acid molecule which encodes a polypeptide comprising the amino acid sequence of SEQ ID NO: 2, SEQ ID NO: 4, SEQ ID NO: 6, SEQ ID NO: 8, SEQ ID NO: 10, or the amino acid sequence encoded by the cDNA insert of the plasmid deposited with the ATCC as Accession Number______.

2. The isolated nucleic acid molecule of claim 1, which is the nucleotide sequence SEQ ID NO: 1, SEQ ID NO: 3, SEQ ID NO: 5, SEQ ID NO: 7, or SEQ ID NO: 9.

3. A host cell which contains the nucleic acid molecule of claim 1.

4. An isolated 25324, 50287, 28899, 47007, or 42967 polypeptide selected from the group consisting of:
a) a polypeptide which is encoded by a nucleic acid molecule comprising a nucleotide sequence which is at least 60% identical to a nucleic acid comprising the nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, or the nucleotide sequence of the DNA insert of the plasmid deposited with ATCC as Accession Number______, or a complement thereof;
b) a naturally occurring allelic variant of a polypeptide comprising the amino acid sequence of SEQ ID NO: 2, SEQ ID NO: 4, SEQ ID NO: 6, SEQ ID NO: 8, SEQ ID NO: 10, or the amino acid sequence encoded by the cDNA insert of the plasmid deposited with the ATCC as Accession Number______, wherein the polypeptide is encoded by a nucleic acid molecule which hybridizes to a nucleic acid molecule comprising SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15 or a complement thereof under stringent conditions;
c) a fragment of a polypeptide comprising the amino acid sequence of SEQ ID NO: 2, SEQ ID NO: 4, SEQ ID NO: 6, SEQ ID NO: 8, SEQ ID NO: 10, or the amino acid sequence encoded by the cDNA insert of the plasmid deposited with the ATCC as Accession Number______, wherein the fragment comprises at least 15 contiguous amino acids of SEQ ID NO: 2, SEQ ID NO: 4, SEQ ID NO: 6, SEQ ID NO: 8, SEQ ID NO: 10; and
d) the amino acid sequence of SEQ ID NO: 2, SEQ ID NO: 4, SEQ ID NO: 6, SEQ ID NO: 8, SEQ ID NO: 10.

5. An antibody which selectively binds to a polypeptide of claim 4.

6. A method for producing a polypeptide selected from the group consisting of:
a) a polypeptide comprising the amino acid sequence of SEQ ID NO: 2, SEQ ID NO: 4, SEQ ID NO: 6, SEQ ID NO: 8, SEQ ID NO: 10, or the amino acid sequence encoded by the cDNA insert of the plasmid deposited with the ATCC as Accession Number______;
b) a polypeptide comprising a fragment of the amino acid sequence of SEQ ID NO: 2, SEQ ID NO: 4, SEQ ID NO: 6, SEQ ID NO: 8, SEQ ID NO: 10, or the amino acid sequence encoded by the cDNA insert of the plasmid deposited with the ATCC as Accession Number______, wherein the fragment comprises at least 15 contiguous amino acids of SEQ ID NO: 2, SEQ ID NO: 4, SEQ ID NO: 6, SEQ ID NO: 8, SEQ ID NO: 10, or the amino acid sequence encoded by the cDNA insert of the plasmid deposited with the ATCC as Accession Number______;
c) a naturally occurring allelic variant of a polypeptide comprising the amino acid sequence of SEQ ID NO: 2, SEQ ID NO: 4, SEQ ID NO: 6, SEQ ID NO: 8, SEQ ID NO: 10, or the amino acid sequence encoded by the cDNA insert of the plasmid deposited with the ATCC as Accession Number______, wherein the polypeptide is encoded by a nucleic acid molecule which hybridizes to a nucleic acid molecule comprising SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, or SEQ ID NO: 15; and
d) the amino acid sequence of SEQ ID NO: 2, SEQ ID NO: 4, SEQ ID NO: 6, SEQ ID NO: 8, or SEQ ID NO: 10;
comprising culturing the host cell of claim 3 under conditions in which the nucleic acid molecule is expressed.

7. A method for detecting the presence of a nucleic acid molecule of claim 1 or a polypeptide encoded by the nucleic acid molecule in a sample, comprising:
a) contacting the sample with a compound which selectively hybridizes to the nucleic acid molecule of claim 1 or binds to the polypeptide encoded by the nucleic acid molecule; and
b) determining whether the compound hybridizes to the nucleic acid or binds to the polypeptide in the sample.

8. A kit comprising a compound which selectively hybindizes to a nucleic acid molecule of claim 1 or binds to a polypeptide encoded by the nucleic acid molecule and instructions for use.

9. A method for identifying a compound which binds to a polypeptide or modulates the activity of the polypeptide of claim 4 comprising the steps of:
a) contacting a polypeptide, or a cell expressing a polypeptide of claim 4 with a test compound; and
b) determining whether the polypeptide binds to the test compound or determining the effect of the test compound on the activity of the polypeptide.

10. A method for modulating the activity of a polypeptide of claim 4 comprising contacting the polypeptide or a cell expressing the polypeptide with a compound which binds to the polypeptide in a sufficient concentration to modulate the activity of the polypeptide.

11. A method of identifying a nucleic acid molecule associated with cancer or a disorder characterized by aberrant angiogenesis comprising:
a) contacting a sample from a subject with or at risk of developing cancer or a disorder associated with aberrant angiogenesis comprising nucleic acid molecules with a hybridization probe comprising at least 25 contiguous nucleotides of SEQ ID NO: 1, SEQ ID NO: 3, SEQ ID NO: 5, SEQ ID NO: 7, SEQ ID NO: 9, defined in claim 2; and
b) detecting the presence of a nucleic acid molecule in the sample that hybridizes to the probe, thereby identifying a nucleic acid molecule associated with cancer or disorder associated with aberrant angiogenesis.

12. A method of identifying a nucleic acid associated with cancer or a disorder characterized by aberrant angiogenesis comprising:
a) contacting a sample from a subject having cancer or a disorder characterized by aberrant angiogenesis or at risk of developing cancer or a disorder associated with aberrant angiogenesis comprising nucleic acid molecules with a first and a second amplification primer, the first primer comprising at least 25 contiguous nucleotides of SEQ ID NO: 1, SEQ ID NO: 3, SEQ ID NO: 5, SEQ ID NO: 7, or SEQ ID NO: 9, defined in claim 2 and the second primer comprising at least 25 contiguous nucleotides from the complement of SEQ ID NO: 1, SEQ ID NO: 3, SEQ ID NO: 5, SEQ ID NO: 7, or SEQ ID NO: 9 respectively;
b) incubating the sample under conditions that allow nucleic acid amplification; and
c) detecting the presence of a nucleic acid molecule in the sample that is amplified, thereby identifying the nucleic acid molecule associated with cancer or a disorder characterized by aberrant angiogenesis.

13. A method of identifying a polypeptide associated with cancer or a disorder characterized by aberrant angiogenesis comprising:
a) contacting a sample comprising polypeptides with a 25324, 50287, 28899, 47007, or 42967 binding partner of the 25324, 50287, 28899, 47007, or 42967 polypeptide respectively defined in claim 4; and
b) detecting the presence of a polypeptide in the sample that binds to the 25324, 50287, 28899, 47007, or 42967 binding partner, thereby identifying the polypeptide associated with cancer or a disorder characterized by aberrant angiogenesis.

14. A method of identifying a subject having cancer or a disorder characterized by aberrant angiogenesis or at risk for developing cancer or a disorder characterized by aberrant angiogenesis comprising:
a) contacting a sample obtained from the subject comprising nucleic acid molecules with a hybridization probe comprising at least 25 contiguous nucleotides of SEQ ID NO: 1, SEQ ID NO: 3, SEQ ID NO: 5, SEQ ID NO: 7, or SEQ ID NO: 9, defined in claim 2; and
b) detecting the presence of a nucleic acid molecule in the sample that hybridizes to the probe, thereby identifying a subject having cancer or a disorder characterized by aberrant angiogenesis or at risk for developing cancer or a disorder characterized by aberrant angiogenesis.

15. A method of identifying a subject having cancer or a disorder characterized by aberrant angiogenesis or at risk for developing cancer or a disorder characterized by aberrant angiogenesis comprising:
a) contacting a sample obtained from the subject comprising nucleic acid molecules with a first and a second amplification primer, the first primer comprising at least 25 contiguous nucleotides of SEQ ID NO: 1, SEQ ID NO: 3, SEQ ID NO: 5, SEQ ID NO: 7, or SEQ ID NO: 9, defined in claim 2 and the second primer comprising at least 25 contiguous nucleotides from the complement of SEQ ID NO: 1, SEQ ID NO: 3, SEQ ID NO: 5, SEQ ID NO: 7, or SEQ ID NO: 9 respectively;
b) incubating the sample under conditions that allow nucleic acid amplification; and
c) detecting the presence of a nucleic acid molecule in the sample that is amplified, thereby identifying a subject having cancer or a disorder characterized by aberrant angiogenesis or at risk for developing cancer or a disorder characterized by aberrant angiogenesis.

16. A method of identifying a subject having cancer or a disorder characterized by aberrant angiogenesis or at risk for developing cancer or a disorder characterized by aberrant angiogenesis comprising:
a) contacting a sample obtained from the subject comprising polypeptides with a 25324, 50287, 28899, 47007, or 42967 binding partner of the 25324, 50287, 28899, 47007, or 42967 polypeptide defined in claim 4; and
b) detecting the presence of a polypeptide in the sample that binds to the 25324, 50287, 28899, 47007, or 42967 binding partner, thereby identifying a subject having cancer or a disorder characterized by aberrant angiogenesis or at risk for developing cancer or a disorder characterized by aberrant angiogenesis.

17. A method for identifying a compound capable of treating cancer or a disorder characterized by aberrant angiogenesis or modulating cellular proliferation and/or differentiation characterized by aberrant 25324, 50287, 28899, 47007, or 42967 nucleic acid expression or 25324, 50287, 28899, 47007, or 42967 polypeptide activity comprising assaying the ability of the compound to modulate 25324, 50287, 28899, 47007, or 42967 nucleic acid expression or 25324, 50287, 28899, 47007, or 42967 polypeptide activity, thereby identifying a compound capable of treating cancer or a disorder characterized by aberrant angiogenesis characterized by aberrant 25324, 50287, 28899, 47007, or 42967 nucleic acid expression or 25324, 50287, 28899, 47007, or 42967 polypeptide activity.

18. The method of claim 17, wherein the cancer or cellular proliferation and/or differentiation is lung, breast, or colon cancer and wherein the disorder characterized by aberrant angiogenesis is brain tumor angiogenesis.

19. A method for treating a subject having cancer, a disorder characterized by aberrant angiogenesis or for modulating cellular proliferation and/or differentiation, or a subject at risk of developing cancer or a disorder characterized by aberrant angiogenesis comprising administering to the subject a 25324, 50287, 28899, 47007, or 42967 modulator of the nucleic acid molecule defined in claim 1 or the polypeptide encoded by the nucleic acid molecule or contacting a cell with a 25324, 50287, 28899, 47007, or 42967 modulator.

20. The method of claim 19, wherein the cancer is lung, breast, or colon cancer and wherein the disorder characterized by aberrant angiogenesis is brain tumor angiogenesis.

21. The method of claim 19, wherein the 25324, 50287, 28899, 47007, or 42967 modulator is
a) a small molecule;
b) peptide;
c) phosphopeptide;
d) anti-25324, -50287, -28899, -47007, or -42967 antibody;
e) a 25324, 50287, 28899, 47007, or 42967 polypeptide comprising the amino acid sequence of SEQ ID NO: 2, or a fragment thereof;
f) a 25324, 50287, 28899, 47007, or 42967 polypeptide comprising an amino acid sequence which is at least 90 percent identical to the amino acid sequence of SEQ ID NO: 2, SEQ ID NO: 4, SEQ ID NO: 6, SEQ ID NO: 8, or SEQ ID NO: 10, wherein the percent identity is calculated using the ALIGN program for comparing amino acid sequences, a PAM120 weight residue table, a gap length penalty of 12, and a gap penalty of 4; or
g) an isolated naturally occurring allelic variant of a polypeptide consisting of the amino acid sequence of SEQ ID NO: 2, SEQ ID NO: 4, SEQ ID NO: 6, SEQ ID NO: 8, or SEQ ID NO: 10, wherein the polypeptide is encoded by a nucleic acid molecule which hybridizes to a complement of a nucleic acid molecule consisting of SEQ ID NO: 1, SEQ ID NO: 3, SEQ ID NO: 5, SEQ ID NO: 7, or SEQ ID NO: 9 at 6X SSC at 45° C., followed by one or more washes in 0.2X SSC, 0.1% SDS at 65° C.

22. The method of claim 19, wherein the 25324, 50287, 28899, 47007, or 42967 modulator is
a) an antisense 25324, 50287, 28899, 47007, or 42967 nucleic acid molecule;
b) is a ribozyme;
c) the nucleotide sequence of SEQ ID NO: 1, SEQ ID NO: 3, SEQ ID NO: 5, SEQ ID NO: 7, SEQ ID NO: 9, or a fragment thereof;
d) a nucleic acid molecule encoding a polypeptide comprising an amino acid sequence which is at least 90 percent identical to the amino acid sequence of SEQ ID NO: 2, SEQ ID NO: 4, SEQ ID NO: 6, SEQ ID NO: 8, or SEQ ID NO: 10, wherein the percent identity is calculated using the ALIGN program for comparing amino acid sequences, a PAM120 weight residue table, a gap length penalty of 12, and a gap penalty of 4;
e) a nucleic acid molecule encoding a naturally occurring allelic variant of a polypeptide comprising the amino acid sequence of SEQ ID NO: 2, SEQ ID NO: 4, SEQ ID NO: 6, SEQ ID NO: 8, or SEQ ID NO: 10, wherein the nucleic acid molecule which hybridizes to a complement of a nucleic acid molecule consisting of SEQ ID NO: 1, SEQ ID NO: 3, SEQ ID NO: 5, SEQ ID NO: 7, or SEQ ID NO: 9, at 6X SSC at 45° C., followed by one or more washes in 0.2X SSC, 0.1% SDS at 65° C.; or
f) a gene therapy vector.

23. A method for evaluating the efficacy of a treatment of cancer, a disorder characterized by aberrant angiogenesis, or aberrant cellular proliferation and/or differentiation in a subject, comprising:
treating a subject with a protocol under evaluation;
assessing the expression level of a 25324, 50287, 28899, 47007, or 42967 nucleic acid molecule defined in claim 1 or 25324, 50287, 28899, 47007, or 42967 polypeptide encoded by the 25324, 50287, 28899, 47007, or 42967 nucleic acid molecule, wherein a change in the expression level of 25324, 50287, 28899, 47007, or 42967 nucleic acid or 25324, 50287, 28899, 47007, or 42967 polypeptide after the treatment, relative to the level before the treatment, is indicative of the efficacy of the treatment of cancer or disorder characterized by aberrant angiogenesis.

24. The method of claim 17, wherein the cancer is lung, breast, or colon cancer and wherein the disorder characterized by aberrant angiogenesis is brain tumor angiogenesis.

25. A method of diagnosing cancer or a disorder characterized by aberrant angiogenesis in a subject, comprising:
evaluating the expression or activity of a 25324, 50287, 28899, 47007, or 42967 nucleic acid molecule defined in claim 1 or a 25324, 50287, 28899, 47007, or 42967 polypeptide encoded by the 25324, 50287, 28899, 47007, or 42967 nucleic acid molecule, such that a difference in the level of 25324, 50287, 28899, 47007, or 42967 nucleic acid or 25324, 50287, 28899, 47007, or 42967 polypeptide relative to a normal subject or a cohort of normal subjects is indicative of cancer or disorder characterized by aberrant angiogenesis.

26. The method defined in claim 25 wherein the cancer is lung, breast, or colon cancer and wherein the disorder characterized by aberrant angiogenesis is brain tumor angiogenesis.
